(19) United States
(12) Patent Application Publication
(10) Pub. No.: US 2024/0240267 A1
(43) Pub. Date: Jul. 18, 2024

(54) ASSAYS FOR DETECTING PATHOGENS

(71) Applicant: Angstrom Bio, Inc., Austin, TX (US)

(72) Inventors: Carlos F. Santos, Austin, TX (US); David J. States, Ann Arbor, MI (US); Jonathan P. Feldmann, Austin, TX (US); Josue D. Moran, Austin, TX (US)

(73) Assignee: Angstrom Bio, Inc., Austin, TX (US)

(21) Appl. No.: 17/907,083

(22) PCT Filed: Mar. 24, 2021
(86) PCT No.: PCT/IB2021/052463

§ 371 Date: Sep. 22, 2022

Related U.S. Application Data
(60) Provisional application No. 62/994,173, filed on Mar. 24, 2020.

(51) Int. Cl. C12Q 1/70; C12Q 1/6844; G16B 30/10

(52) U.S. Cl. CPC C12Q 1/701; C12Q 1/6844; G16B 30/10; C12Q 2600/156; C12Q 2600/166

(57) ABSTRACT

Provided herein are compositions and methods for identifying target nucleic acids that are determinants of pathogenic infections. The multiplexed methods provided herein simultaneously detect target proteins such as IgG and IgM immunoglobulins that are indicative of one or more pathogenic infections, from several distinct biological samples. Also provided herein are methods for detecting sequence variants in a nucleic acid sample.

### CROSS-REFERENCE TO RELATED APPLICATIONS

This application claims the benefit of priority to U.S. Provisional Application No. 62/994,173, filed on Mar. 24, 2020. The entire contents of the foregoing application are hereby incorporated herein by reference.

### SEQUENCE LISTING

The instant application contains a Sequence Listing which has been submitted electronically in ASCII format and are hereby incorporated by reference in its entirety. The ASCII copy, created on Mar. 22, 2021, is named A109922_1010WO_SL.txt and is 58,177 bytes in size.

### FIELD OF THE INVENTION

Provided herein are arrays and methods for detecting pathogens such as coronaviruses (e.g., 229E, NL63, OC43, HKU1, MERS-CoV, SARS-CoV and SARS-CoV-2) and/or other viruses such as the influenza viruses (e.g., influenza virus A, influenza virus B, influenza virus C, and influenza virus D), bacteria (e.g., Mycobacterium, Streptococcus, Pseudomonas, Shigella, Campylobacter, Chlamydia and Salmonella) in a sample, e.g., a biological sample (e.g., a blood sample, an oral sample, a nasal sample, or a tissue sample).

### BACKGROUND OF THE INVENTION

Early detection of a disease is often critical for successful control and treatment of the disease. Providing accurate, high speed, and low cost blood analysis, infection diagnosis, pathogen detection, or other biological or chemical analyte detection remains a major challenge for health providers and hazardous response teams.

A case in point, is the diagnosis of infectious diseases such as viral infections caused by coronaviruses, which are large, enveloped RNA viruses, that cause highly prevalent diseases in humans and domestic animals. Coronaviruses are transmitted by aerosols of respiratory secretions, by the fecal-oral route, and by mechanical transmission. In many cases, the patients infected with the virus are asymptomatic and in other cases, infections cause a mild, self-limited disease (classical “cold” or upset stomach), and there may be rare neurological complications. The novel SARS-CoV-2 (COVID19) virus appears to be localized to the pulmonary cells of the lower respiratory tract, cause severe respiratory complications leading to death in select patient populations.

SARS-CoV-2 possesses a deadly combination of high infectiousness and virulence, coupled with a variable, but extended period of asymptomatic presentation in a large fraction of patients, that has overwhelmed healthcare systems worldwide. Reports from China, Iran, Spain, and Italy demonstrate that an inability to control the spread of the disease in the early weeks of a localized outbreak leads to a flood of patients who require intensive care for acute respiratory distress or otherwise life-threatening symptoms, which can rapidly overwhelm local and regional healthcare system capacity and send mortality rates soaring. The COVID-19 outbreak has been declared a public health emergency of international concern by the World Health Organization, causing significant impact on people's lives, families and communities. Thus, the ability to diagnose COVID-19 and opportunistic infections early should lead to more effective therapy decisions and improved outcomes for patients. Further, detection of a population production of neutralizing antibodies, could lead to identification of health risks of a population to the particular pathogen.

Sophisticated analyte detection systems are available, but they are bulky, costly, and require extensive raining to calibrate, operate and maintain. Rapid diagnostic test can provide the advantages of low per-test cost, simple operation, and minimal or no required instrumentation, but there are also significant limitations. Rapid diagnostic test is often configured to test only a single sample for a single analyte, so multiple devices are needed to support co-infection testing, which can be prohibitively expensive and impractical.

A need exists for the development of a massively parallel and rapid diagnostic tests that can detect and distinguish between pathogens or determinants of infection in a patient clinical sample accurately and efficiently.

### SUMMARY OF THE INVENTION

The compositions and methods as described herein are useful for the simultaneous rapid detection of pathogens from multiple samples. The present disclosure also provides methods for detecting sequence variants in a nucleic acid sample. The compositions, arrays, systems and methods described herein combine the simplicity of a PCR or a proximity ligation assay to generate uniquely barcoded amplicons with the parallel sequencing of the plurality of amplicons, and are able to provide source identifying information in addition to identifying the presence or absence of one or more analytes (e.g., polynucleotides and/or proteins) from biological samples.

In one aspect, the present disclosure provides a method for identifying at least one target nucleic acid. The method comprises the steps of a) obtaining a plurality of biological samples from a plurality of subjects, b) obtaining total nucleic acid from each of the biological samples, c) subjecting the plurality of polynucleotides to amplification using an amplification mixture to produce a plurality of amplicons, d) detecting each of the plurality of amplicons and e) determining a category of the plurality of amplicons. In some embodiments, the plurality of polynucleotides comprise RNA molecules, and step b) further comprises obtaining cDNA reverse-transcribed from the RNA or reverse-transcribing cDNA from the RNA before performing the amplification in step c). In a particular embodiment, the plurality of polynucleotides in step b) comprises RNA molecules, and a reverse transcriptase is added in step b) to obtain a plurality of cDNAs that will be subjected to amplification in step c). In some embodiments, the plurality of polynucleotides in step b) further comprises DNA molecules.

In many embodiments of the methods described herein, the target nucleic acid is obtained from a sample comprising one or more pathogens selected from the group consisting of a RNA virus, a DNA virus, a fungus, a parasite and a bacterium. In some embodiments, the pathogen is selected from a group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta. Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A. Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae. Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis, or is another potentially novel or uncharacterized pathogen sharing distinctive nucleic acid sequences with a pathogen in the aforementioned group. In one embodiment, the pathogen is an RNA virus. In a particular embodiment, the RNA virus is SARS-CoV-2.

In many embodiments of the methods described herein, the sample is selected from the group consisting of blood, mucus, saliva, sweat, tears, fluids accumulating in a bodily cavity, urine, ejaculate, vaginal secretion, cerebrospinal fluid, lymph, feces, sputum, decomposition fluid, vomit, sweat, breast milk, serum, and plasma. In one embodiment, the sample is saliva.

In one embodiment of the method, the plurality of polynucleotides are subjected to amplification using an amplification mixture to produce a plurality of amplicons. In many embodiments of the methods described herein, the amplification is a polymerase chain reaction amplification. In some embodiments, the amplification is a rolling circle amplification. In one embodiment of the method, the amplification mixture comprises a plurality of primers, the forward primers and the reverse primers. In one embodiment, the method described herein provides for the amplification of the cDNAs using an amplification mixture comprising unique sets of forward primers and reverse primers. In some embodiments, the primers comprise a set of nucleotides that are complementary to each of the plurality of cDNAs and at least one unique nucleotide barcode sequence. In some embodiments, the plurality of primers comprises at least 96 different barcoded primers. In some embodiments, the method comprises a first unique barcode sequence that identifies the biological sample obtained from the specific subject.

In many embodiments of the methods described herein, the pair of adapter sequences separate the first unique barcode sequence and its reverse complement from a second unique barcode sequence and its reverse complement. In many embodiments of the methods described herein, the pair of adapter sequences flank the first unique barcode sequence and its reverse complement.

In many embodiments of the methods described herein, detecting comprises sequencing the plurality of amplicons comprising the pair of adapter sequences and the first unique barcode sequence and its reverse complement. In many other embodiments of the methods described herein, detecting comprises sequencing the plurality of amplicons comprising the pair of adapter sequences, the first unique barcode sequence and its reverse complement, and the second unique barcode sequence and its reverse complement. In the embodiments of the methods described herein, detecting is performed by reading a sequencing data file with a suite of programs. In one embodiment, the suite of programs comprises HMMER/Infernal alignments. In one embodiment, the sequencing data file is a FASTA/FASTQ formatted file.

In many embodiments, the method further comprises sequencing at least one positive control sample, that is a target nucleic acid. In some embodiments, the method further comprises sequencing at least one positive control sample that is a Bacteriophage MS2. In some embodiments, the method further comprises sequencing at least one positive control sample that is a MS2 template nucleic acid. In some embodiments, the method further comprises sequencing at least one positive control sample that is a RNAseP or another non-pathogen gene. In some embodiments, the method further comprises sequencing at least one positive control sample that is a is a nucleic acid from a human housekeeping gene GAPDH or beta-actin.

In many embodiments, the method comprises identifying two or more target nucleic acids. In some embodiments, the two or more target nucleic acids are pathogenic determinants, or encode for pathogenic determinants, of a single pathogen. In one embodiment, the two or more target nucleic acids are pathogenic determinants, or encode for pathogenic determinants, of a virus. In one embodiment, the virus is SARS-CoV-2. In some embodiments, the pathogenic determinants are selected from the group consisting of a spike protein (S), a receptor-binding domain (RBD), a S1 protein, a S2 protein, E gene, S gene, Orflab gene, N-terminal Spike protein domain, a whole protein (S1+S2), and a nucleocapsid (N) protein.

In some embodiments, the two or more target nucleic acids are pathogenic determinants, or encode for pathogenic determinants, of at least two different pathogens selected from a group consisting of a RNA virus, a DNA virus, a fungus, a parasite and a bacterium. In one embodiment, the two different RNA viruses are SARS-CoV-2 and Influenza.

In another aspect, the disclosure provides a multiplex of array for detecting at least one target protein from multiple samples. In one embodiment, the multiplex array comprises a plurality of capture agents bound to a plurality of uniquely labeled beads with each uniquely labeled bead comprising a plurality of unique capture agents. In the embodiments described herein, the multiplex array comprises at least one first oligonucleotide sequence that is designed to be bound to at least one bead, at least one secondary antibody conjugated with a second oligonucleotide sequence and at least one unique nucleotide barcode sequence in the circular amplicon. In many embodiments of the array described herein, the bead is coated with an antigen that specifically binds at least one target protein. In some embodiments of the array described herein the second oligonucleotide sequence is designed to be amplified to form a circular amplicon when the second oligonucleotide sequence is in close proximity to the first oligonucleotide sequence. In some embodiments, the first oligonucleotide sequence, or the second oligonucleotide sequence, or both, comprise at least one unique barcode sequence. In some embodiments, the first oligonucleotide sequence is covalently bound to a polypeptide coated on the bead. In some embodiments, the multiplex of arrays comprise the first oligonucleotide sequence that is covalently bound to an antibody or an antibody fragment, where the antibody or the antibody fragment bind to a polypeptide coated on the bead. In one embodiment, the multiplex array comprises at least 96 different barcode sequences in the circular amplicon.

In one aspect, the present disclosure provides a method for at least one infection in a plurality of biological samples. The method comprises the first step of incubating a plurality of biological samples with a plurality of beads in the multiplex of array described herein under conditions sufficient for at least one target protein to bind to the unique capture agent of at least one of the beads. In the second step of the method. The beads are washed to remove any proteins that do not bind to the unique capture agents. The next step involves incubating the beads with a plurality of secondary antibodies under conditions where each of the plurality of the secondary antibodies forms a complex with at least one target protein, such that plurality of complexes corresponding to the number of the secondary antibodies bound to the plurality of target proteins, are formed. In the next step, the beads are washed again to remove any secondary antibodies that do not form the complex. In the sixth step, the plurality of complexes are incubated under conditions to allow hybridization of each of the second oligonucleotide sequence to each of the first oligonucleotide sequence such that they form a circular amplicon, such that plurality of amplicons are generated corresponding to the number of the plurality of complexes. The seventh step of the method involves subjecting the plurality of circular amplicons to amplification. In the eighth step, the beads are pooled in the array and the plurality of amplicons are simultaneously detected by high throughput sequencing of the unique barcoded amplicons. In the final step, the category of the plurality of amplicons is determined. Determining the category of each the plurality of amplicons comprising the polynucleotides from the target amplified region indicates infection in the corresponding biological sample.

In some embodiments, the method described herein is used for the identification of pathogenic determinants (e.g., bacterial, fungal, parasitic and/or viral infections) in one or more samples. In other embodiments, the method simultaneously detects target proteins such as IgG and IgM immunoglobulins that are indicative of one or more pathogenic infections. In some embodiments, the antibody or the antibody fragment detected by the method described herein bind specifically to one or more antigens from pathogens including Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus. Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43. Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1. SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito. South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype. Tick-borne encephalitis complex (flavi) virus Siberian subtype. Tobacco mosaic virus, Torque teno virus, Trichuris trichiura, Trypanosoma brucci, Trypanosoma cruzi, Variola major virus (Smallpox virus), Variola minor virus (Alastrim), Venezuelan equine encephalitis virus, Wuchereria bancrofti, Yersinia pestis, or is another potentially novel or uncharacterized pathogen sharing distinctive nucleic acid sequences with a pathogen in the aforementioned group.

In one embodiment, the antibody or the antibody fragment binds specifically to an antigen from SAR-CoV-2. In some embodiments, the antibody or the antibody fragment binds specifically to an antigen selected from the group consisting of a spike protein (S), a receptor-binding domain (RBD), a SI protein, a S2 protein, E gene, S gene, Orflab gene, N-terminal Spike protein domain, a whole protein (S1+S2), and a nucleocapsid (N) protein.

In many embodiments of the methods described herein, the sample is selected from the group consisting of blood, mucus, saliva, sweat, tears, fluids accumulating in a bodily cavity, urine, ejaculate, vaginal secretion, cerebrospinal fluid, lymph, feces, sputum, decomposition fluid, vomit, sweat, breast milk, serum, and plasma. In one embodiment, the sample is saliva. In one embodiment, the sample is blood.

In another aspect, the disclosure provides a method for detecting sequence variants in a nucleic acid sample. The first step involves performing an amplification reaction with the sample of nucleic acid with an amplification mixture to produce a plurality of amplicons. The second step is to detect sequence variations comprises detecting, and optionally quantitating, the plurality of amplicons. The third step of the method comprises a step of determining a category of the plurality of amplicons. The fourth step of the method is directed to the detection of sequence variations. In many embodiments of the method described herein, the amplification mixture comprises the nucleic acid sample, a plurality of primers, a first unique barcode sequence and its reverse complement, and a first pair of adapter sequence. In some embodiments, each of the plurality of the primers comprise a set of nucleotides that are complementary to each of the polynucleotides that they bind to. In some embodiments, the first unique barcode sequence and its reverse complement identify the sample obtained from a specific subject. In some embodiments, the pair of adapter sequences flanks the first unique barcode sequence and its reverse complement. In some embodiments, the plurality of amplicons comprises polynucleotides from a target amplified region or a control region.

In many embodiments of the methods described herein, the second step comprises sequencing each of the plurality of amplicons comprising the first pair of adapter sequences and the first unique barcode sequence and its reverse complement. In some embodiments, the second step comprises sequencing each of the plurality of amplicons comprising the first pair of adapter sequences, the first unique barcode sequence and its reverse complement, the second unique barcode sequence and its reverse complement. In one embodiment, the first pair of adapter sequences separate the first unique barcode sequence and its reverse complement from a second unique barcode sequence and its reverse complement.

In many embodiments of the methods described herein, the detecting in the second step is performed by reading a sequencing data file with a suite of programs. In some embodiments, the sequencing data file is in a FASTA/FASTQ format. In some embodiments, the suite of programs comprises HMMER/Infernal alignment engines.

In many embodiments of the methods described herein, the detecting in the fourth step comprises performing a sequence alignment (e.g., multiple sequence alignment) with one or more reference sequences. In some embodiments, the sequence alignment is performed by a HMM profile Hidden Markov Model (HMM) engine, a covariance model (CM) engine or a combination thereof.

In some embodiments, the method comprising correlating the sequence variants with a diagnosis or a prognosis of an infection. In some embodiments, the infection is caused by one or more pathogens selected from the group consisting of a RNA virus, a DNA virus, a fungus, a parasite and a bacterium. In some embodiments, the pathogen is selected from a group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis. Bacillus anthracis Pasteur strain, Bacillus cereus Biovar anthracis, Brucella abortus, Brucella melitensis, Brucella suis, Burkholderia mallei. Burkholderia pseudomallei. Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii. Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis, or is another potentially novel or uncharacterized pathogen sharing distinctive nucleic acid sequences with a pathogen in the aforementioned group.

In one embodiment, the pathogen is SARS-CoV-2. In some embodiments, the sequence variants are in a region encoding an antigen selected from the group consisting of a spike protein (S), a receptor-binding domain (RBD), a SI protein, a S2 protein, E gene, S gene, Orflab gene, N-terminal Spike protein domain, a whole protein (S1+S2), and a nucleocapsid (N) protein. In some embodiments, the sequence variants comprise mutations selected from a group consisting of T95I, D253G, L452R, E484K, S477N, N501Y D614G and A701V.

In many embodiments of the methods described herein, detecting the plurality of amplicons comprises obtaining a pooled sequence dataset of the plurality of amplicons, performing base calling, aligning the sequence data of the plurality of amplicons to a pre-defined, annotated HMM or CM gene model, assigning a rank (e.g., a probability score or a bit score) to each of the HMM/CM alignments, filtering the sequence data to obtain a positionally annotated sequence alignments and denoting the barcode(s) within each amplicon as well as the location of the barcode and the adapter within the amplicon's sequence. In all of embodiments of the methods described herein, the foregoing steps are performed using a suitably programmed computer. In some embodiments of the methods described herein, base calling is performed with a high-accuracy ONT GPU-based base caller, yielding raw FASTA/FASTQ files. In these embodiments, raw files are the aligned by a profile HMM engine and/or a CM engine. In some embodiments, the HMM engine comprises a HMMER software program that yields a plurality of sequence alignments. In some embodiments, the HMMER program and/or the CM engine assign a per-nucleotide annotation for one or more sequence feature selected from a group consisting of the barcode, the target amplified region, the primer, and the adapter. In one embodiment, the plurality of sequence alignments comprises annotations for the first unique barcode sequence and its reverse complement.

In many embodiments of the methods described herein, filtering comprises assigning a pass score or a fail score to the sequence alignments. In these embodiments, the sequence alignments are assigned a passing score if they pass a minimum Levenshtein distance score relative to a set of reference barcoded sequences and if they pass a minimum bitscore threshold for alignments. In some embodiments, the sequence alignments with a passing score are stored in a central database. In some embodiments, the sequence alignments with the passing score correspond to a direct quantitative representation of a pathogen load in the sample.

In many embodiments of the method, the database comprises information of a unique barcode assigned to a sample collection tube, information of a set of at least 96 unique well barcodes, information of a set of at least 96 unique plate barcodes, information of a set of sequence data from the plurality of amplicons and a report. In one embodiment, the report comprises source identifying information of each subject and information on whether the subject is positive or negative for the presence of the target protein. In one embodiment, the report is provided to corresponding subjects, or to a clinic or to a physician.

In yet another aspect, the present disclosure provides compositions comprising an amplicon. In many embodiments described herein, the amplicon comprises a first unique barcode sequence and its reverse complement, a pair of target-specific primers, a target amplified region and a first pair of adapter sequences. In some embodiments, the pair of target specific primers is made up of a forward primer and a reverse primer, each having sequences complementary to the priming sites in a target amplified region (e.g., a region of a viral genome). In many embodiments, each of the forward primer and the reverse primer flanks the target amplified region and is in turn flanked by the first unique nucleotide barcode sequence and its reverse complement, the first unique barcode sequence and its reverse complement are flanked by first pair of adapter sequences. In some embodiments, the amplicon further comprising a second unique barcode sequence and its reverse complement and a second pair of adapter sequences, where the second unique barcode sequence and its reverse complement and the second pair of adapter sequences, are ligated to the amplicon. In one embodiment, first pair of adapter sequences are flanked by the second pair of adapter sequences, and where the second pair of adapter sequences are flanked by the second unique barcode sequence and its reverse complement.

In some embodiments, the target amplified region is amplified from a genomic region of a pathogen encoding for a gene or protein, where the pathogen is selected from the group consisting Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei. Trypanosoma cruzi, Variola major virus (Smallpox virus), Variola minor virus (Alastrim), Venezuelan equine encephalitis virus, Wuchereria bancrofti, Yersinia pestis, or is another potentially novel or uncharacterized pathogen sharing distinctive nucleic acid sequences with a pathogen in the aforementioned group.

In one embodiment, the pathogen is SARS-CoV-2. In some embodiments, the sequence variants are in a region encoding an antigen selected from the group consisting of a spike protein (S), a receptor-binding domain (RBD), a S1 protein, a S2 protein, E gene, S gene, Orflab gene, N-terminal Spike protein domain, a whole protein (S1+S2), and a nucleocapsid (N) protein. In one embodiment, the target amplified region is amplified from a region encoding the S protein. In one embodiment, the target amplified region is amplified from a region encoding the RBD of the S protein. In one embodiment, the target amplified region is amplified from a region encoding the N protein.

In some embodiments, the unique barcode sequences and their reverse complements have a maximal Levenshtein distance from all other barcodes. In some embodiments, the unique barcode sequences comprise any one of the polynucleotide sequences set forth in SEQ ID NOs.:23-118. In some embodiments, the pair of target-specific primers is selected from a group of forward and reverse primers consisting of Forward Primer: GACCCCAAAATCAGCGAAAT (SEQ ID NO.:3) and Reverse Primer: TCTGGTTACTGCCAGTTGAATCTG (SEQ ID NO.:4); Forward Primer: TTACAAACATTGGCCGCAAA (SEQ ID NO.:5) and Reverse Primer: GCGCGACATTCCGAAGAA (SEQ ID NO.:6); Forward Primer: GGGAGCCTTGAATACACCAAAA (SEQ ID NO.:7) and Reverse Primer: TGTAGCACGATTGCAGCATTG (SEQ ID NO.:8); Forward Primer: GTGARATGGTCATGTGTGGCGG (SEQ ID NO.:9) and Reverse Primer: CARATGTTAAASACACTATTAGCATA (SEQ ID NO.:10); Forward Primer: ACAGCTACGTTAATAGTTAATAGCGT (SEQ ID NO.:11) and Reverse Primer: ATATTGCAGCAGTACGCACACA (SEQ ID NO.:12); Forward Primer: CCCTGTGGGTTTTACACTTAA (SEQ ID NO.:13) and Reverse Primer: ACGATTGTGCATCAGCTGA (SEQ ID NO.:14); Forward Primer: GTACTCATTCGTTTCGGAAGAG (SEQ ID NO.:15) and Reverse Primer: CCAGAAGATCAGGAACTCTAGA (SEQ ID NO.:16); Forward Primer: GGGGAACTTCTCCTGCTAGAAT (SEQ ID NO.:17) and Reverse Primer: CAGACATTIGCTCTCAAGCTG (SEQ ID NO.:18); and Forward Primer: AGATTTGCiACCTGCGAGCG (SEQ ID NO.:19) and Reverse Primer: GAGCCYGCTGTCTCCACAAGT (SEQ ID NO.:20).

In some embodiments, the first pair of adapter sequences and the second pairs of adapter sequences are identical comprise between 10 to 15 nucleotides. In one embodiment, the pair of adapter sequences comprise 10 nucleotides. In one embodiment, the pair of adapter sequences comprise polynucleotide sequence as set forth in ACACTGACGACATGGTTCTACA (SEQ ID NO.:21) and TACGGTAGCAGAGACTTGGTCT (SEQ ID NO.:22).

### BRIEF SUMMARY OF THE FIGURES

FIG. 1: Overview of the reverse transcriptase assay to detect and/or identify the presence of at least one target nucleic acid from a pathogen.

FIG. 2: Overview of the serology assay to detect and/or identify the presence of at least one at least one target protein from one or more biological sample(s)

FIG. 3: Schematic of a bioinformatics pipeline. FIG. 3 discloses SEQ ID NO: 133.

FIG. 4 shows an exemplary amplicon generated by the amplification of the target N1 protein in the SARS-Cov2 genome using primers about 20 nucleobases in length ligated to a barcoded sequence that is unique for each patient sample. FIG. 4 discloses SEQ ID NOS 134-151, respectively, in order of appearance.

FIGS. 5A-B Exemplary alignment files with annotations for the unique barcode sequence, adapter sequence and the target amplified region. FIG. 5A shows sequence labeling and scoring data of an exemplary target E-Guelph protein from the SARS-Cov2 genome (SEQ ID NOS 152-155, respectively, in order of appearance). FIG. 5B shows sequence labeling and scoring data of an exemplary target RNAseP from the SARS-Cov2 genome (SEQ ID NOS 156-159, respectively, in order of appearance).

FIG. 6 shows multiplexed PCR and sequencing results from the SARS-CoV-2 gene targets, demonstrating excellent amplification and high alignment scores.

FIG. 7 shows multiplexed PCR and sequencing results with high reproducibility obtained across the multiple sequencing runs.

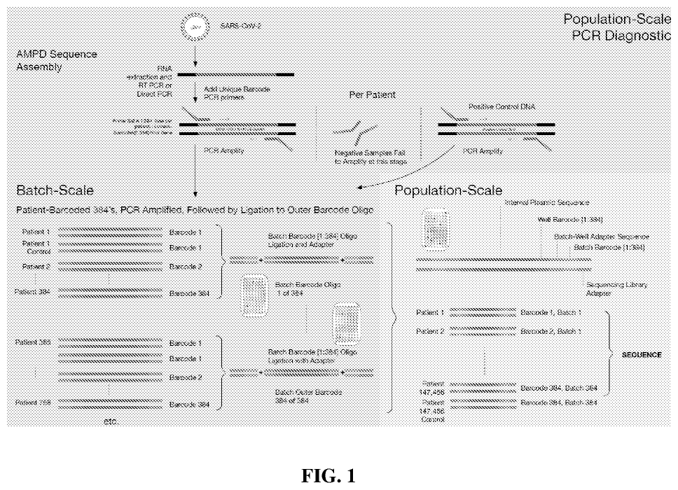

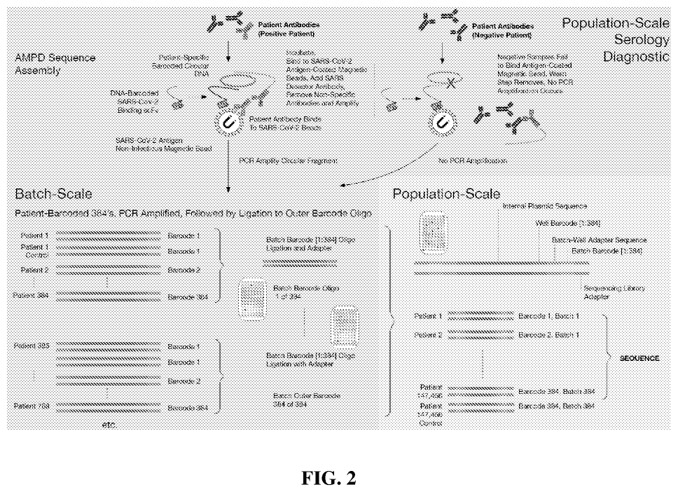

### DETAILED DESCRIPTION

The compositions and methods as described herein are useful for the simultaneous rapid detection of pathogens from multiple samples. The present disclosure provides multiplex assays that employ hundreds or more of target specific primers containing unique detectable nucleotide barcode sequences in a single reaction to detect the presence of specific analytes (e.g., viral particles, antibodies against a pathogenic determinant from a pathogen) in one or more samples. The present disclosure also provides methods for detecting sequence variants in a nucleic acid sample. The compositions, arrays, systems and methods described herein combine the simplicity of a PCR or a proximity ligation assay to generate uniquely barcoded amplicons with the parallel sequencing of the plurality of amplicons, and are able to provide source identifying information in addition to identifying the presence or absence of one or more analytes (e.g., polynucleotides and/or proteins) from biological samples.

### I. Definitions

The term “amplicon” refers to a nucleic acid product of a PCR reaction. Amplicons provided herein contain barcode sequences flanking the sequence of interest (e.g., viral sequence). The amplicon can be double-stranded or single-stranded, and can include the separated component strands obtained by denaturing a double-stranded amplification product. In certain embodiments, the amplicon of one amplification cycle can serve as a template in a subsequent amplification cycle.

The term “analyte” refers to a substance to be detected or assayed by the methods described herein. Typical analytes may include, but are not limited to peptides, proteins (e.g., antibody, fragments of antibody, scFv), nucleic acids, small molecules, including organic and inorganic molecules, viruses and other microorganisms, cells etc., as well as fragments and products thereof, such that any analyte can be any substance or entity that can participate in a specific binding pair interaction, e.g., for which epitopes (i.e., attachment sites), binding members or receptors (such as antibodies) can be developed.

As used herein, the term “binding domain” refers to a moiety that is selected from a group of an antibody, antibody derivative, a peptide, a protein or a nucleic acid aptamer. The term “antibody” refers to a protein consisting of one or more polypeptides substantially encoded by all or part of the recognized immunoglobulin genes. The recognized immunoglobulin genes, for example in humans, include the kappa (κ), lambda (l), and heavy chain genetic loci, which together comprise the myriad variable region genes, and the constant region genes mu (μ), delta (δ), gamma (γ), sigma (ε), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. Antibody fragments are known in the art and include, but are not limited to. Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies).

The term “amplification” refers to the process in which “replication” is repeated in cyclic process such that the number of copies of the nucleic acid sequence is increased in either a linear or logarithmic fashion. Such replication processes may include but are not limited to, for example, rolling circle amplification (RCA), Polymerase Chain Reaction (PCR). RCA driven by DNA polymerase can amplify circular oligonucleotide probes with either linear or geometric kinetics under isothermal conditions, as described in Lizardi et al., Nature Genet. 19: 225-232 (1998); U.S. Pat. Nos. 5,854,033 and 6,143,495; PCT Application No. WO 97/19193), all of the references are incorporated in their entirety. In some embodiments. RCA involves circularization of a probe molecule hybridized to a target sequence and subsequent rolling circle amplification of the circular probe as described in U.S. Pat. Nos. 5,854,033 and 6,143,495; PCT Application No. WO 97/19193. Very high yields of amplified products can be obtained with rolling circle amplification, as described in U.S. Pat. Nos. 5,854,033 and 6,143,495; PCT Application No. WO 97/19193, and Dean et al., Genome Research 11:1095-1099 (2001). The references provided herein are incorporated in their entirety. By “amplicon” is meant a polynucleotide generated during the amplification of a polynucleotide of interest. In one example, an amplicon is generated during a polymerase chain reaction.

As used herein, a “biological sample” refers to a sample of tissue or fluid isolated from a subject (or animal), which in the context of the disclosure generally refers to samples suspected of containing nucleic acid from the pathogens (e.g., viral RNA), viral particles (e.g., viral particles of SARS-CoV-2 virus) and/or antibodies or fragment thereof that bind specifically with one or more pathogenic antigens. The samples, after optional processing, can be analyzed in an in vitro assay. Typical samples of interest include, but are not necessarily limited to, respiratory secretions (e.g., samples obtained from fluids or tissue of nasal passages, lung, and the like), blood, plasma, serum, blood cells, fecal matter, urine, tears, saliva, milk, organs, biopsies, and secretions of the intestinal and respiratory tracts. Samples also include samples of in vitro cell culture constituents including but not limited to conditioned media resulting from the growth of cells and tissues in culture medium. e.g., recombinant cells, and cell components.

As used herein, “barcode sequence”, or “detectable barcode sequence” or “molecular tags” or “barcode label”, or grammatical equivalents thereof, is meant a moiety (e.g., nucleotide sequence of 3-15 nucleotides) that can act as a source identifier and/or facilitate the recognition of a nucleotide sequence (e.g., DNA, RNA). In certain embodiments, each original DNA or RNA molecule is attached to a unique sequence barcode and such a sequence can be traced to a unique source sequence or a set of unique sequences after the completion of the assays described herein. It is generally understood that sequence reads having different barcodes represent different original molecules, while sequence reads having the same barcode are results of PCR duplication from one original molecule. The target quantification can also be achieved by counting the number of unique molecular barcodes in the reads rather than counting the number of total reads, as total read counts are more likely skewed for targets by non-uniform amplification. By “unique barcode”, “distinct barcode”, or grammatical equivalents thereof is meant that a first barcode can be distinguished from a second barcode (or all other barcodes) in a detection assay either by its detection characteristic (e.g., unique sequence) or its intensity/concentration/absolute amount.

Throughout the specification, abbreviations are used to refer to nucleotides (also referred to as bases), including abbreviations that refer to multiple nucleotides. As used herein, G=guanine, A=adenine. T=thymine, C=cytosine, and U=uracil. Nucleotides can be referred to throughout using lower or upper case letters.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A. T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the disclosure. Usually two sequences are sufficiently complementary when at least about 85% (preferably at least about 90%, and most preferably at least about 95%) of the nucleotides share base pair organization over a defined length of the molecule.

The term “Levenshtein distance score” as used herein is the score assigned to each barcode the greatest Levenshtein distance to all other barcodes, and sorting in descending Levenshtein distance. As used herein, the term “Levenshtein distance”, corresponds to the measure of the difference between two sequences. For example, the Levenshtein distance between a first and a second barcode sequence corresponds to the number of single nucleotide changes required to change the first barcode sequence into the second barcode sequence. Levenshtein distances can be averaged. In some embodiments, the junctions are designed so as to have an average of 2 or higher junction distance. In some embodiments, the design of the barcode sequences that result in the maximal Levenshtein distance is selected.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides. As such, the term “nucleic acid” includes polymers in which the conventional backbone of a polynucleotide has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Unless specifically indicated otherwise, there is no intended distinction in length between the terms “polynucleotide,” “oligonucleotide,” “nucleic acid” and “nucleic acid molecule” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

The terms “multiplex” or “multiplexing” refer to simultaneous detection of multiple samples combined into a single reaction. Multiplexing with multiple unique barcode sequences allows individualized detection and source identification of several samples in one experiment. The term “multiplex PCR” as used herein refers to an assay that provides for simultaneous amplification and detection of two or more target nucleic acids within the same reaction vessel. Each amplification reaction is primed using a distinct primer pair. In some embodiments, at least one primer of each primer pair is labeled with a detectable moiety. In some embodiments, a multiplex reaction may further include specific probes for each target nucleic acid. In some embodiments, the specific probes are detectably labeled with different detectable moieties.

The term “primer” or “oligonucleotide primer” as used herein, refers to an oligonucleotide which acts to initiate synthesis of a complementary nucleic acid strand when placed under conditions in which synthesis of a primer extension product is induced. e.g., in the presence of nucleotides and a polymerization-inducing agent such as a DNA or RNA polymerase and at suitable temperature, pH, metal concentration, and salt concentration. Primers are generally of a length compatible with their use in synthesis of primer extension products, and are usually in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

The term “primer” refers to a polynucleotide, generally an oligonucleotide comprising a “target” binding portion that is typically about 12 to about 35 nucleotides long, that is designed to selectively hybridize with a target nucleic acid flanking sequence or to a corresponding primer binding site of an amplification product under typical stringency conditions; and serve as the initiation point for the synthesis of a nucleotide sequence that is complementary to the corresponding polynucleotide template from its 3′-end.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The terms “forward” and “reverse” when used in reference to the primers of a primer pair indicate the relative orientation of the primers on a polynucleotide sequence. For example, the “reverse” primer is typically designed to anneal with the downstream primer binding site at or near the “3-end” of the template polynucleotide in a 5′ to 3′ orientation, right to left. The corresponding “forward primer” is designed to anneal with the complement of the upstream primer-binding site at or near the “5′-end” of the polynucleotide in a 5′ to 3′ “forward” orientation, left to right. A “primer pair” described herein comprises a forward primer and a corresponding reverse primer.

The term “probe”, as used herein, refers to a polynucleotide that comprises a portion that is designed to hybridize in a sequence-specific manner with a complementary probe binding site on a particular nucleic acid sequence, for example, an amplicon. The sequence-specific portions of probes and primers described herein are of sufficient length to permit specific annealing to complementary sequences in target nucleic acids and desired amplicons.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The phrase “conditions to allow hybridization” refers to conditions under which a primer will hybridize preferentially to, or specifically bind to, its complementary binding partner, and to a lesser extent to, or not at all to, other sequences. An example of a condition to allow hybridization is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. In some embodiments, conditions to allow hybridization are stringent hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment described herein.

By “bind” or “bound” is meant that the molecule binds preferentially to the target of interest or binds with greater affinity to the target than to other molecules. For example, beads coated with antigen will bind to a specific bind antibody and not to any immunoglobulin molecule.

The term “identifying” includes any form of measurement, and includes determining the presence, absence or amount of the analyte to be detected. In one embodiment, the analyte is an COVID 19 polynucleotide or other RNA viral polynucleotide. The terms “determining”, “detecting”, “measuring”. “evaluating”, “assessing” and “assaying” are used interchangeably and include quantitative and qualitative determinations. Identifying may be relative or absolute. “Identifying a” includes determining the amount of something present, and/or determining whether it is present or absent. As used herein, the terms “determining,” “measuring,” and “assessing,” and “assaying” are used interchangeably and include both quantitative and qualitative determinations.

The terms “high throughput sequencing” “high throughput, massively parallel sequencing”, “third-generation sequencing”, or “nanopore sequencing” as used herein refers to sequencing methods that can generate multiple sequencing reactions of clonally amplified molecules and of single nucleic acid molecules in parallel. This allows increased throughput and yield of data. These methods are also known in the art as next generation sequencing (NGS) methods. NGS methods include, for example, sequencing-by-synthesis using reversible dye terminators, and sequencing-by-ligation, and nanopore sequencing. Non-limiting examples of commonly used NGS platforms include miRNA BeadArray (Illumina, Inc.). Roche 454™ GS FLX™-Titanium (Roche Diagnostics), ABI SOLiD™ System (Applied Biosystems, Foster City, CA), and HeliScope™ Sequencing System (Helicos Biosciences Corp., Cambridge MA), and Oxford Nanopore Sequencers.

The term “read” as used herein generally refers to the data comprising the sequence composition obtained from a single nucleic acid template molecule or a population of a plurality of substantially identical copies of the template nucleic acid molecule.

By “reverse transcriptase” is meant an enzyme that replicates a primed single-stranded RNA template strand into a complementary DNA strand in the presence of deoxyribonulceotides and permissive reaction medium comprising, but not limited to, a buffer (pH 7.0-9.0), sodium and/or potassium ions and magnesium ions. As is apparent to one skilled in the art, concentration and pH ranges of a permissive reaction media may vary in regard to a particular reverse transcriptase enzyme. Examples of suitable “reverse transcriptases” well known in the art, but not limited to, are MmLV reverse transcriptase and its commercial derivatives “Superscript I, II and III” (Life Technologies), “MaxiScript” (Fermentas), RSV reverse transcriptase and its commercial derivative “OmniScript” (Qiagen), AMV reverse transcriptase and its commercial derivative “Thermoscript” (Sigma-Aldrich).

“Coronavirus” as used herein refers to a genus of the family Coronaviridae. The coronaviruses are large, enveloped, positive-stranded RNA viruses, which replicate by a unique mechanism that results in a high frequency of recombination.

The term “COVID 19”, also referred to as “Wuhan-hu-1.” “Severe acute respiratory syndrome coronavirus 2 isolate, SARS-CoV-2,” refers to a virus that belongs to a family of viruses, i.e., the Coronaviridae, a group IV ((+) ssRNA) virus of the genus betacoronavirus following the nomenclature of the Coronavirus Study group (de Groot 2013).

The term “Middle East Respiratory Syndrome Coronavirus” is also abbreviated herein as MERS, is a group IV ((+) ssRNA) virus of the genus betacoronavirus following the nomenclature of the Coronavirus Study group (de Groot 2013). This virus was first described as human coronavirus EMC in 2012 by Zaki et al. (2012), Bermingham et al. (2012), van Boheemen et al. (2012) as well as Müller et al. 2012. The complete genome of the human betacoronavirus 2c EMC/2012 has been deposited under the GenBank accession number JX869059.2

The term “Severe acute respiratory syndrome coronavirus, SARS-CoV.” refers to a virus that belongs to a family of viruses, i.e., the Coronaviridae, a group IV ((+) ssRNA) virus of the genus betacoronavirus following the nomenclature of the Coronavirus Study group (de Groot 2013). The SARS-CoV genomic RNA is ^{˜}29,700 base pairs in length and has 14 open reading frames (orfs), encoding the replicase, spike, membrane, envelop and nucleocapsid (N) which are similar to other coronaviruses, and several other unique proteins (Marra et al, 2003; Rota et al, 2003). The SARS-CoV genome length RNA is likely packaged by a 50-kDa-nucleocapsid protein (N) [8]. As with other coronaviruses, the virion contains several viral structural proteins including the ^{˜}140 kDa spike glycoprotein (S), a 23 kDa membrane glycoprotein (M) and a ^{˜}10 kDa protein (E).

### II. Amplicons

In one aspect, the present disclosure provides compositions comprising an amplicon. In many embodiments described herein, the amplicon comprises a first unique barcode sequence and its reverse complement, a pair of target-specific primers, a target amplified region and a first pair of adapter sequences. The pair of target specific primers is made up of a forward primer and a reverse primer, each having sequences complementary to the priming sites in a target amplified region (e.g., a region of a viral genome). In many embodiments, each of the forward primer and the reverse primer flanks the target amplified region and is in turn flanked by the first unique nucleotide barcode sequence and its reverse complement, the first unique barcode sequence and its reverse complement are flanked by first pair of adapter sequences. The spacer sequence, also referred herein as an adapter sequence or an adapter, typically comprises a conserved sequence of a defined length (e.g., 10 nucleotides). Exemplary amplicon structure from 5′ to 3′ is [forward_adapter]-[first unique barcode sequence]-[forward primer]-[target amplified region]-[reverse primer]-[first unique barcode (reverse complemented)]-[reverse_adapter]. In some embodiments, a second set of unique barcodes, the second unique barcode sequence and its reverse complement, can be ligated. Exemplary amplicon structure with second set of barcodes from 5′ to 3′ is [second unique barcode sequence]-[second forward_adapter]-[first forward_adapter]-[first unique barcode sequence]-[forward primer]-[target amplified region]-[reverse primer]-[first unique barcode (reverse complemented)J-[first reverse_adapter]-[second reverse_adapter]-[second unique barcode (reverse complemented)]. Exemplary barcoded forward and reverse primer sequences for SARS-Cov-2 PCR target −N1 gene, are shown below.

Barcoded Forward Primer-
(SEQ. ID NO. 1)
TAACTTGGTCGACCCCAAAATCAGCGAAAT
Barcoded Reverse Primer-
(SEQ. ID NO. 2)
GTCTAAGTTGACCGTCATTGGTCTATTGAACCAG

In the disclosure provided herein, at least one primer may be used (e.g., for sequencing a sample from a subject, or to prepare a library). In some embodiments, one primer may be used. In some embodiments, more than one primer may be used. For example, 1, 2, 3. 4, 5, 6, 7, 8, 9, or 10 primers may be used. In some embodiments, more than 10 primers may be used. For example, a first, a second, a third, a fourth, a fifth, a sixth, a seventh, an eighth, a ninth and/or a tenth primer may be used. In some embodiments, a primer may contain a desired sequence. In some embodiments, a primer may contain more than one desired sequence. For example, a desired sequence may be a pre-determined sequence, a complementary sequence, a known sequence, a binding sequence, a universal sequence, or a detection sequence. In some embodiments, a pre-determined sequence may be a universal sequence.

In some embodiments, a polynucleotide (e.g., target sequence or a sequence in the target amplified region) may be contacted with at least one primer containing a desired sequence. In some embodiments, the primer may be, but not limited to, hybridized or annealed to the polynucleotide. For example, the primer with the desired sequence (e.g., predetermined target sequence) may be used to amplify the polynucleotide using an enzyme. For example, the enzyme may be a polymerase (e.g., a Taq polymerase). In some embodiments, the primer containing a predetermined sequence may be annealed or hybridized to the 3′ end or the 5′ end of the polynucleotide. In some embodiments, more than one pre-determined sequence may be annealed or hybridized to the polynucleotide. For example, a first pre-determined sequence may be annealed or hybridized to one end of the polynucleotide and a second pre-determined sequence may be annealed or hybridized to the other end of the polynucleotide. In some embodiments, the first pre-determined sequence may be complementary to the second pre-determined sequence. In some embodiments, the first pre-determined sequence may be reverse complementary to the second pre-determined sequence. In some embodiments, the first pre-determined sequence may not be complementary to the second pre-determined sequence.

Non-limiting exemplary primer pairs that are useful in the compositions and the methods provided herein include, Forward Primer: GACCCCAAAATCAGCGAAAT (SEQ ID NO.:3) and Reverse Primer: TCTGGTTACTGCCAGTTGAATCTG (SEQ ID NO.:4); Forward Primer: TTACAAACATTGGCCGCAAA (SEQ ID NO.:5) and Reverse Primer: GCGCGACATTCCGAAGAA (SEQ ID NO.:6); Forward Primer: GGCGAGCCTTGAATACACCAAAA (SEQ ID NO.:7) and Reverse Primer: TGTAGCACGATTGCAGCATTG (SEQ ID NO.:8); Forward Primer: GTGARATGGTCATGTGTGGCGG (SEQ ID NO.:9) and Reverse Primer: CARATGTTAAASACACTATTAGCATA (SEQ ID NO.:10); Forward Primer: ACAGGTACGTTAATAGTTAATAGCGT (SEQ ID NO.:11) and Reverse Primer: ATATTGCAGCAGTACGCACACA (SEQ ID NO.:12); Forward Primer: CCCTGTGGGTTTTACACTTAA (SEQ ID NO.:13) and Reverse Primer: ACGATTGTGCATCAGCTGA (SEQ ID NO.:14); Forward Primer: GTACTCATTCGTTTCGCGAAGAG (SEQ ID NO.:15) and Reverse Primer: CCAGAAGATCAGGAACTCTAGA (SEQ ID NO.:16); Forward Primer: GGGGAACTTCTCCTGCTAGAAT (SEQ ID NO.:17) and Reverse Primer: CAGACATTTTGCTCTCAAGCTG (SEQ ID NO.:18); and Forward Primer: AGATTTGGACCTGCGAGCG (SEQ ID NO.:19) and Reverse Primer: GAGCGGCTGTCTCCACAAGT (SEQ ID NO.:20).

In some embodiments, a single stage barcoding procedure with a first unique barcode sequence and its reverse complement is used. In such embodiments, the first unique barcode and its reverse complement and the first pair of adapter sequences are introduced by the primers used in the amplification process. In these embodiments, the first pair of adapter sequences has an invariant sequence with at least 10 to 15 nucleotides. In one embodiment, the invariant adapter sequences have 10 nucleotides. In a specific embodiment, the invariant adapter sequences comprise polynucleotide sequence as set forth in ACACTGACGACATGGTTCTACA (SEQ ID NO.:21) and TACGGTAGCAGAGACTTGGTCT (SEQ ID NO.:22). Other invariant adapter sequences can be generated and fall within the scope of this disclosure. In some other embodiments, a single stage barcoding procedure with a first unique barcode sequence and a second unique reverse complement is used. In such embodiments, the first unique barcode, the second unique reverse complement and the first pair of adapter sequences are introduced by the primers used in the amplification process.

In other embodiments, a two stage barcoding procedure with a first unique barcode sequence and its reverse complement and a second unique barcode sequence and its reverse complement are used. In these embodiments, the first unique barcode sequence and its reverse complement, and the adapter sequence (e.g., first pair of adapter sequences) is introduced by the primer used in the amplification process. The second set of barcodes (e.g. the barcodes used to track samples pooled from a stage 1 plate, second unique barcode sequence) are ligated to the ends of the amplicon. As a result, the invariant adapter sequence will be located between the two barcodes. This avoids ambiguity that might result from having the two barcodes immediately adjacent to each other. In some embodiments, a two stage barcoding procedure with two distinct inner barcode sequences are used. In some other embodiments, a two stage barcoding procedure with two distinct outer barcode sequences are used. In yet another embodiment, a two stage barcoding procedure with two distinct inner barcode sequences and two distinct outer barcode sequences are used. In some embodiments with distinct inner and outer barcodes, all the four (two inner and two outer) barcodes are distinct.

As described below in detail, when a two stage barcode procedure is used, the sequence used to generate the Hidden Markov Model (HMM) or covariance model (CM) incorporates ambiguity sequences on both sides of the invariant spacer sequence so that the alignment of the sequence read to the statistical model correctly annotates both sets of barcodes. An alternative is to create HMM or CM models for each of the second stage barcodes used and to use the quality of the match to these models to assign the identity of the second stage barcode. In many embodiments with the two stage barcoding, there are two pairs of adapter sequences, the first pair of adapter sequences and the second pair of adapter sequences. In some embodiments, the first and second pair of adapter sequences are identical with an invariant sequence having at least 10 to 15 nucleotides. In one embodiment, the invariant adapter sequences have 10 nucleotides. In a specific embodiment, the invariant adapter sequences comprise polynucleotide sequence as set forth in ACACTGACGACATGGTTCTACA (SEQ ID NO.:21) and TACGGTAGCAGAGACTTGGTCT (SEQ ID NO.:22). Other invariant adapter sequences can be generated and fall within the scope of this disclosure.

In many embodiments, an invariant adapter sequence is at the 5′ end of each primer. As a result, each amplicon sequence begins at the 5′ end with a copy of the adapter sequence from the forward strand primer and at the 3′ end has a reverse complemented sequence of the adapter derived from the reverse strand primer. Without wishing to be bound by theory, these adapter sequences serve two purposes. First, they aid in segmenting long reads into constituent amplicon sequences, and second, they anchor the position of the unique barcode sequences in the HMM or CM alignment described below, allowing to reliably annotate the positions of the unique barcode sequences.

In some embodiments, the outer barcodes (e.g., plate/batch identifiers) are added to the barcoded amplicons, typically using a ligation reaction. Ligated outer barcodes avoids cross-amplification inherent to 2nd PCR stage-based amplifications. The inner barcode, in some instances, is a patient or well specific barcode to annotate a specific sample from a plurality of distinct samples in a plate with at least 96 wells. The outer barcode can denote a specific batch or can be a plate identifier when there is a plurality of distinct samples in distinct plates with multiple batches of plates. Barcode sequences and primers are selected from a very large, validated IDT barcode library that has been screened for secondary structure interactions, resulting in a highly optimized, error tolerant barcode design. In the embodiments of the compositions and methods provided herein that comprise barcode sequences, Levenshtein distance (LD) barcode optimization is undertaken to ensure sequencing error tolerance and maximal distinguishability. First, from a set of candidate barcodes, the Levenshtein distance between every barcode to every other barcode is calculated. The barcodes are then ranked by assigning to each barcode the greatest Levenshtein distance to all other barcodes, and sorting in descending Levenshtein distance. Then a desired number of barcodes are selected (e.g. 96, or 384) from a group with barcode candidates from the ranked list, having the maximal LD separating them from other barcodes.

Non-limiting exemplary barcode sequences are provided in Table 1. The 96 barcode sequences in Table 1 (selected from within the 3000+ total barcodes) are maximally Levenshtein-distance separated. In some embodiments, 384 maximally Levenshtein-distance separated barcode sequences are selected. The selection of barcode sequences is done algorithmically and yields different results depending on the selection size. In many embodiments of the methods and compositions provided herein, the number of barcode sequences selected is based on the size of the barcode pool that the primers are assembled from.

TABLE 1
Non-limiting exemplary barcode sequences that
are maximally Levenshtein-distance separated

| | SEQ ID |
|---|---|
| Barcode Sequence | NOs.: |
| CTTACGAACT | 23 |
| TGCAACACTA | 24 |
| CCAGTATTCT | 25 |
| GCGCCATATA | 26 |
| CTGGAGAATA | 27 |
| AACCGGCCTT | 28 |
| AGAACCGGAC | 29 |
| TTGGCTCCAA | 30 |
| AAGCGGTGAA | 31 |
| CGACGTACAC | 32 |
| ATTCTTGCCT | 33 |
| CGGCTAACCA | 34 |
| CGTAAGAGAA | 35 |
| GATGAAGGAT | 36 |
| GAATGTCCAA | 37 |
| TCACCAGGAC | 38 |
| AAGTTAGGAC | 39 |
| AAGAGCACCT | 40 |
| GTGCCACAAC | 41 |
| AGGTTCACAC | 42 |
| TTGAAGGAAC | 43 |
| TTAACACGCT | 44 |
| TGCTGGTATA | 45 |
| TGGAGCCGAT | 46 |
| ATTACCTCTC | 47 |
| CCTTATGGTT | 48 |
| GAGGAGATAA | 49 |
| CTTGCACTCA | 50 |
| GTTCCTAGAT | 51 |
| GTAAGGTGTT | 52 |
| CGGTGTTCCT | 53 |
| GATATACGCA | 54 |
| TGGCTATTAC | 55 |
| TGTTCTCTTC | 56 |
| TTCCATGGTA | 57 |
| ACCTCCTTCT | 58 |
| GAGTTGGCTC | 59 |
| CAAGTGCGTC | 60 |
| CCTTGGTGCA | 61 |
| AGGAATTGTC | 62 |
| CCTGTGTAAC | 63 |
| CAACAAGGCA | 64 |
| TTGTGGCGCA | 65 |
| AATTCGCGCT | 66 |
| CGGTAATATC | 67 |
| ATTGGTGGTA | 68 |
| GCCTCAACTC | 69 |
| AGAACGCAAT | 70 |
| ACAAGACAAC | 71 |
| CTGTCAAGTT | 72 |
| ACCATTACCA | 73 |
| GTGGTGAGAC | 74 |
| TTAGACCTTC | 75 |
| GATACGACTT | 76 |
| CGGATTGGAA | 77 |
| AGCGAGGATT | 78 |
| GCCAGAATAA | 79 |
| TGTTCAGCAA | 80 |
| AACACCGGTT | 81 |
| CTCCAGCTTC | 82 |
| TCTTACCGAA | 83 |
| CTCCAATCCT | 84 |
| ACACTCTCAA | 85 |
| GACCGCGTTA | 86 |
| TACGGCAGAA | 87 |
| CACATGGAAT | 88 |
| TATGCCGGCA | 89 |
| ATCGCCACCT | 90 |
| AAGCCGAATC | 91 |
| AGAGCTTGTT | 92 |
| TCCTGACAAT | 93 |
| AGAGGCCTCA | 94 |
| TTCGTTGAAC | 95 |
| GAGTACTGTA | 96 |
| GCACAGTCCT | 97 |
| CTCATGTCTA | 98 |
| TCGATTGCTT | 99 |
| AATAGAGCCA | 100 |
| ATCCTCCGAC | 101 |
| GAAGCTTACT | 102 |
| TGCCTTGGCT | 103 |
| ATTGGAACAC | 104 |
| AACCAACCAA | 105 |
| AGGTTACCTT | 106 |
| TATGGTTCTC | 107 |
| GAACAATCAC | 108 |
| CGGTGCTTAA | 109 |
| CCGCGTCATT | 110 |
| CCAACAGATT | 111 |
| CACACTTATC | 112 |
| CTAATCCACA | 113 |
| GTCTTCCATT | 114 |
| TCTCGGAGTT | 115 |
| CTCTCGATAA | 116 |
| GCCAATGTTC | 117 |
| TCACTTCGCA | 118 |

Non-limiting exemplary outer barcode sequences are provided in Table 2. The exemplary barcode sequences in Table 2 are maximally Levenshtein-distance separated.

TABLE 2
Non-limiting exemplary outer barcode sequences
that are maximally Levenshtein-distance separated

| | SEQ ID | Reverse complement of | SEQ ID |
|---|---|---|---|
| Barcode Sequence | NOs.: | barcode Sequence | NOs.: |
| CACAAAGACACCGACAACTTTCTT | 160 | AAGAAAGTTGTCGGTGTCTTTGTG | 184 |
| ACAGACGACTACAAACGGAATCGA | 161 | TCGATTCCGTTTGTAGTCGTCTGT | 185 |
| CCTGGTAACTGGGACACAAGACTC | 162 | GAGTCTTGTGTCCCAGTTACCAGG | 186 |
| TAGGGAAACACGATAGAATCCGAA | 163 | TTCGGATTCTATCGTGTTTCCCTA | 187 |
| AAGGTTACACAAACCCTGGACAAG | 164 | CTTGTCCAGGGTTTGTGTAACCTT | 188 |
| GACTACTTTCTGCCTTTGCGAGAA | 165 | TTCTCGCAAAGGCAGAAAGTAGTC | 189 |
| AAGGATTCATTCCCACGGTAACAC | 166 | GTGTTACCGTGGGAATGAATCCTT | 190 |
| ACGTAACTTGGTTTGTTCCCTGAA | 167 | TTCAGGGAACAAACCAAGTTACGT | 191 |
| AACCAAGACTCGCTGTGCCTAGTT | 168 | AACTAGGCACAGCGAGTCTTGGTT | 192 |
| GAGAGGACAAAGGTTTCAACGCTT | 169 | AAGCGTTGAAACCTTTGTCCTCTC | 193 |
| TCCATTCCCTCCGATAGATGAAAC | 170 | GTTTCATCTATCGGAGGGAATGGA | 194 |
| TCCGATTCTGCTTCTTTCTACCTG | 171 | CAGGTAGAAAGAAGCAGAATCGGA | 195 |
| TCACACGAGTATGGAAGTCGTTCT | 172 | AGAACGACTTCCATACTCGTGTGA | 196 |
| TCTATGGGTCCCAAGAGACTCGTT | 173 | AACGAGTCTCTTGGGACCCATAGA | 197 |
| CAGTGGTGTTAGCGAGGTAGACCT | 174 | AGGTCTACCTCGCTAACACCACTG | 198 |
| AGTACGAACCACTGTCAGTTGACG | 175 | CGTCAACTGACAGTGGTTCGTACT | 199 |
| ATCAGAGGTACTTTCCTGGAGGGT | 176 | ACCCTCCAGGAAAGTACCTCTGAT | 200 |
| GCCTATCTAGGTTGTTGGGTTTGG | 177 | CCAAACCCAACAACCTAGATAGGC | 201 |
| ATCTCTTGACACTGCACGAGGAAC | 178 | GTTCCTCGTGCAGTGTCAAGAGAT | 202 |
| ATGAGTTCTCGTAACAGGACGCAA | 179 | TTGCGTCCTGTTACGAGAACTCAT | 203 |
| TAGAGAACGGACAATGAGAGGCTC | 180 | GAGCCTCTCATTGTCCGTTCTCTA | 204 |
| CGTACTTTGATACATGGCAGTGGT | 181 | ACCACTGCCATGTATCAAAGTACG | 205 |
| CGAGGAGGTTCACTGGGTAGTAAG | 182 | CTTACTACCCAGTGAACCTCCTCG | 206 |
| CTAACCCATCATGCAGAACTATGC | 183 | GCATAGTTCTGCATGATGGGTTAG | 207 |

29

The barcodes and barcoded primers can be made specific to any organism, including but not limited to humans, mammals and even plants. The barcodes can be specifically designed to annotate for human pathogens (e.g., Sar-CoV-2) and pathogens of all kinds of important veterinary diseases (e.g. bovine diarrhea, Johne's disease, pig influenza, etc.). The barcodes can facilitate individual detection of infected animals within a herd, as long as the animals are labelled to each sample and barcode-primed appropriately.

In many embodiments described herein, the target amplified region is amplified from a genomic region of a pathogen encoding for a gene or protein. The pathogen is selected from the group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43. Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus. Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis. Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii. Rift Valley fever virus, Rinderpest virus, Rotavirus A, Rotavirus B, Rotavirus C, Rotavirus G2, Rubella virus, SARS-associated coronavirus (SARS-CoV). SARS-CoV-1, SARS-CoV-2, Schistosoma haematobium, Schistosoma japonicum, Schistosoma mansoni, Sheep pox virus, South American Haemorrhagic Fever virus Chapare. South American Haemorrhagic Fever virus Guanarito, South American Haemorrhagic Fever virus Junin, South American Haemorrhagic Fever virus Machupo. South American Haemorrhagic Fever virus Sabia, Staphylococcus aureus. Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype. Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above, or is another potentially novel or uncharacterized pathogen sharing distinctive nucleic acid sequences with a pathogen in the aforementioned group.

In one embodiment, the target amplified region corresponds to a specific viral genome region of SARS-CoV-2. Non-limiting examples of genomic region encoding for protein from which the target amplified region is amplified includes a region encoding an antigen selected from the group consisting of a spike protein (S), a receptor-binding domain (RBD), a S1 protein, a S2 protein. E gene, S gene, Orflab gene, N-terminal Spike protein domain, a whole protein (S1+S2), and a nucleocapsid (N) protein. In one embodiment, the target amplified region is amplified from a region encoding the S protein. In another embodiment, the target amplified region is amplified from a region encoding the RBD of the S protein. In yet another embodiment, the target amplified region is amplified from a region encoding the N protein.

In the amplicon constructs provided herein, further spacer sequences (e.g., adapter sequence) can be applied outside the barcodes (e.g., flanking the first set of barcode sequence), and these spacer sequences are important for later amplicon region annotation (per-nucleotide annotations of regions of interest by a profile Hidden Markov Model or Covariance Model alignment algorithm). In some embodiments, the amplicons include only two spacer sequences. In other embodiments, the amplicons included at least four or more spacer sequences. In some embodiments, the adapter sequence was included to allow addition of a second unique barcode sequence to each of the plurality of amplicons. In some instances, the adapter sequence acts as a marker during sequence reads to signal the end of a barcode sequence and/or the beginning of the next barcode sequence. In many of the embodiments described herein, the spacer sequences are conserved. In particular embodiments, all the spacer sequences in the barcoded amplicons were identical sequences. In some embodiments, the adapter sequence comprises at least 10 nucleotides. In some other embodiments, the adapter sequence comprises between 10 to 15 nucleotides. In one embodiment, the adapter sequence comprises 10 nucleotides.

### III. Sequencing, HMM and CM Engines

In many embodiments of the compositions and methods described herein, high throughput sequencing is used. In some embodiments, high throughput sequencing is used to detect the unique barcodes in the amplicons. In some other embodiments, high throughput sequencing is used to detect the sequence variants within the target amplified regions of the amplicons. Any high throughput sequencing platforms known in the art may be used to sequence the sequencing libraries prepared as described herein (see, Myllykangas et al.. Bioinformatics for High Throughput Sequencing, Rodriguez-Ezpeleta et al. (eds.), Springer Science+Business Media. LLC, 2012, pages 11-25). Exemplary high throughput DNA sequencing systems include, but are not limited to, the Oxford Nanpore platform, including MinION and PromethION instruments, the GS FLX sequencing system originally developed by 454 Life Sciences and later acquired by Roche (Basel, Switzerland), Genome Analyzer developed by Solexa and later acquired by Illumina Inc. (San Diego, CA) (see, Bentley, Curr Opin Genet Dev 16:545-52, 2006; Bentley et al.. Nature 456:53-59, 2008), the SOLiD sequence system by Life Technologies (Foster City, CA) (see, Smith et al., Nucleic Acid Res 38: e 142, 201 0; Valouev et al., Genome Res 18:1051-63, 2008), CGA developed by Complete Genomics and acquired by BGI (see, Drmanac et al., Science 327:78-81, 2010), PacBio RS sequencing technology developed by Pacific Biosciences (Menlo Park, CA) (see, Eid et al., Science 323: 133-8, 2009), and Ion Torrent developed by Life Technologies Corporation (see, U.S. Patent Application Publication Nos. 2009/0026082; 2010/01 37143; and 2010/0282617). The Oxford Nanopore DNA sequencing systems used in the methods described herein are more suited to rapidly and accurately read amplicons that are routinely over 250 bp in length. The Illumina sequencing system may not be as suited to the methods described herein compared to the Oxford Nanopore DNA sequencing systems (e.g., ONT MinION or GridION) due to long processing time and sequencing-by-synthesis, yielding relatively short reads.

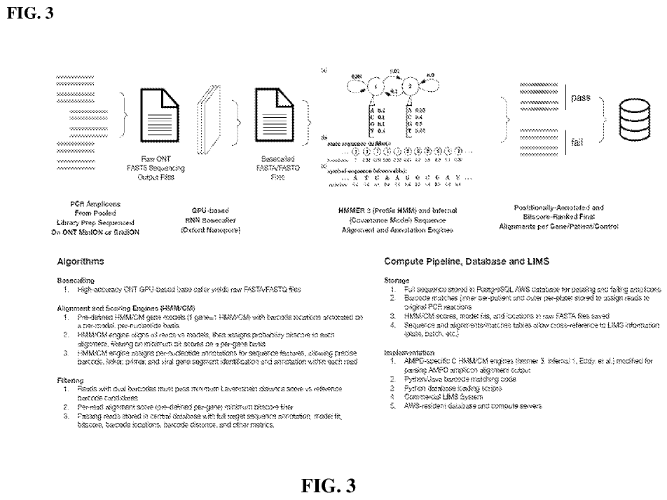

A non-limiting exemplary bioinformatics pipeline applied in the methods described herein overview shown in FIG. 3. In step 1 of the bioinformatics pipeline, the PCR amplicons from pooled library preparations are sequenced on ONT MinION or GridION to obtain raw ONT FAST5 sequencing output files. In the next step, high-accuracy ONT GPU-based base caller yields raw FASTA/FASTQ files. The next step subjects the FASTA/FASTQ files to the HMMER3 and CM sequence alignment and annotation engines that applies the statistical pattern classification algorithm to generate the consensus sequence by a) maximizing a likelihood based upon the replicate sequence reads, and/or b) using a context dependent alignment model parameter based upon a whole genome multiple sequence alignment. In the final filtering step, reads with dual barcodes must pass minimum Leventshein distance score vs reference barcode candidates. Passing reads are stored in a central database with full target sequence annotation, model fit, bitscore, barcode locations, barcode distance, and other metrics.

Various methods for providing the sequence reads of the plurality of amplicons include repeatedly sequencing a single molecule or sequencing multiple molecules, each of which comprises at least a portion of the region of interest. Alignment of the multiple sequence reads of the plurality of amplicons generally involves one or more multiple sequence alignment algorithms, e.g., that use a reference sequence or that use a de novo assembly routine. In certain embodiments, methods of determining a consensus sequence were applied iteratively for a given plurality of barcode sequence reads, e.g. using different subsets of reads for different iterations of the methods. Such subsets can be chosen by various criteria, e.g., quality thresholds of varying stringency. Combined target+linker+barcoded primers yield full-length, error-tolerant amplicons that both improve read quality and call accuracy, and that take full advantage of nanopore sequencing's long-read capability.

In many embodiments of the compositions and the methods described herein, barcode identification and recovery from each amplicon from among plurality of sequences, require the use of statistical pattern classification algorithm that applies one or more likelihood models, error models, probabilistic graph models (e.g., an all path probabilistic alignment). Profile hidden markov model aligners (HMMER) and optionally Covariance Models (Infernal) were used as bioinformatics tools to allow for efficient barcode identification and recovery from each amplicon. HMMER and CM facilitate labelling every nucleotide (even in a noisy sequence read filled with sequencer errors like insertions, substitutions, and deletions) with a maximum likelihood of it being part of a given feature. For example, the barcode regions are clearly defined and the probabilistic aligner assigns a “region” annotation to each letter in a sequence coming out of the instrument. This allows for the identification of distinct primers, and also allows identification of malformed amplicons (e.g. primer-dimer pairs). HMMER assigns a bitscore which corresponds to a likelihood of a given alignment given the length of the match, independent of the search database. These scores are important to rank amplicons for each sample by their quality and allowing to overcome the nanopore instrument sequencing errors. These algorithms are critical for the ability to be able to demultiplex samples. The amplicon sequences provided herein were designed for optimal computational annotation and scoring via profile HMM's and CM's.

The statistical models such as a profile Hidden Markov Model (pHMM or HMM for short) or covariance model (CM) alignment engine were used in the methods described herein to (1) segment long reads into their constituent amplicon sequence, (2) identify high-quality matching sequencer-derived amplicon sequences matching a pre-defined sequence model, (3) rank amplicon sequences according to the exactness (“quality”) of their alignment (also known as match) versus the pre-defined sequence model, and (4) identify internal artificial sequence domains or features within the amplicons according to corresponding (pre-annotated) features in the pre-defined sequence model.

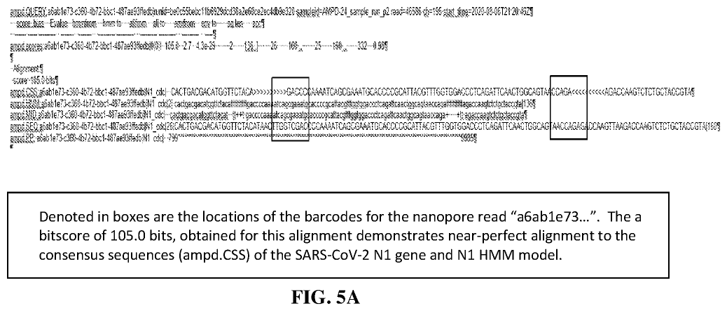

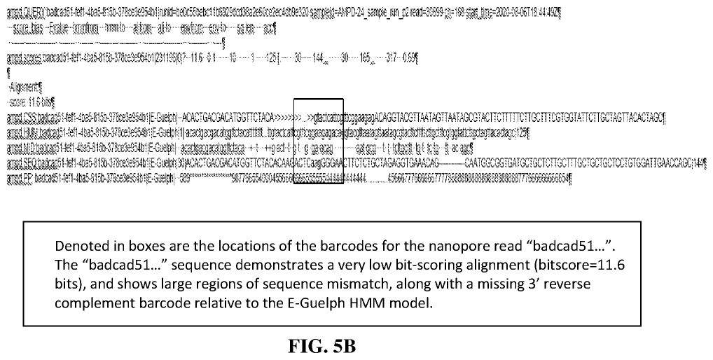

In many embodiments of the methods described herein, detecting the plurality of amplicons comprises obtaining a pooled sequence dataset of the plurality of amplicons, performing base calling, aligning the sequence data of the plurality of amplicons to a pre-defined, annotated HMM or CM gene model, assigning a rank (e.g., a probability score or a bit score) to each of the HMM/CM alignments, filtering the sequence data to obtain a positionally annotated sequence alignments and denoting the barcode(s) within each amplicon as well as the location of the barcode and the adapter within the amplicon's sequence. In all of embodiments of the methods described herein, the foregoing steps are performed using a suitably programmed computer. In some embodiments of the methods described herein, base calling is performed with a high-accuracy ONT GPU-based base caller, yielding raw FASTA/FASTQ files. In these embodiments, raw files are the aligned by a profile HMM engine and/or a CM engine. The HMM engine comprises a HMMER software program that yields a plurality of sequence alignments. The HMMER program is fairly quick to run relative to the computation exhaustive CM engine but either programs assign a per-nucleotide annotation for one or more sequence feature selected from a group consisting of the barcode, the target amplified region, the primer, and the adapter. Exemplary alignment files are shown in FIGS. 5A-B with annotations for the unique barcode sequence, adapter sequence and the target amplified region.

In many embodiments of the methods described herein, filtering comprises assigning a pass score or a fail score to the sequence alignments. The sequence alignments are assigned a passing score if they pass a minimum Levenshtein distance score relative to a set of reference barcoded sequences and if they pass a minimum bitscore threshold for alignments. The sequence alignments with a passing score are typically stored in a central database. In many instances, sequence alignments with the passing score correspond to a direct quantitative representation of a pathogen load in the sample. The database described herein generally has information of a unique barcode assigned to a sample collection tube, information of a set of at least 96 unique well barcodes, information of a set of at least 96 unique plate barcodes, information of a set of sequence data from the plurality of amplicons and a report. The report comprises source identifying information of each subject and information on whether the subject is positive or negative for the presence of the target protein. The report can be provided to corresponding subjects, or to a clinic or to a physician.

In many embodiments of the methods described herein, during the course of library preparation for nanopore sequencing, there may be one or more ligation steps resulting in high molecular weight concatemers containing multiple amplicons. The nanopore instrument reads these concatemers as a single long read, sometimes running to tens of thousands of nucleotides in length and containing many individual amplicons. The sequencer also reads individual non-ligated amplicons which are also part of this pool. The HMM or CM statistical models are used to segment these reads into their constituent amplicon sequences which are individually analyzed. In many embodiments of the methods described herein, primer design includes an invariant adapter or spacer sequence at the 5′ end of each primer. As a result, each amplicon sequence will begin at the 5′ end with a copy of the spacer sequence from the forward strand primer and at the 3′ end will have a reverse complemented sequence of the spacer derived from the reverse strand primer. These adapter or spacer sequences serve two purposes. First, they aid in segmenting long reads into constituent amplicon sequences, and second, they anchor the position of the barcode sequence in the HMM or CM alignment allowing us to reliably annotate the barcode sequence position.

The alignment engine (based on hmmer [see, S. R. Eddy, “Profile Hidden Markov Models,” Bioinformatics Review, Vol. 14, no 9, 1998, pages 755-763] for HMMs or Infernal [Nawrocki2009] for CMs) reads a file containing sequence and annotations for targets. In an exemplary embodiments of the methods described herein, the targets are selected from various regions of SARS-CoV-2 genes N and E genes, human gene RNAseP, beta-actin, and a region of Bacteriophage MS2, and/or TM3 is used as a control.

In particular embodiments, the statistical pattern classification algorithm applies a dynamic Bayesian network, e.g., a profile Hidden Markov Model (profile HMM), a Covariance Model (CM). Briefly, pre-defined HMM/CM gene models (1 gene=1 HMM/CM) with barcode locations were annotated on a per-model, per-nucleotide basis. HMM/CM engine aligns all reads vs models, then assigns probability bitscore to each alignment, filtering on minimum bit scores on a per-gene basis. HMM/CM engine assigns per-nucleotide annotations for sequence features, allowing precise barcode, linker, primer, and viral gene segment identification and annotation within each read. The alignment engine then builds an internal statistical model for each of the model sequences provided, and then searches the total output of the nanopore sequencing run for matches to these models. For each candidate alignment thereby identified, the software outputs a report showing the nanopore read identifier, the HMM/CM model matched, the alignment obtained (including gaps, deletions, substitutions, etc.), the probability score, the bitscore (related to the probability score, but independent of the target database search size), and other details including position of the model match within the raw nanopore sequence read, etc. Hundreds of thousands to millions such alignments (and therefore, candidate amplicon sequences) are generated on a typical run. The annotation of the barcode regions with specific symbols (e.g., denoted by ‘>’ and ‘<’ characters in the output file) is critical, as the reports are read with a set of scripts that identify the barcode region and extract the actual bases in the given alignment as the amplicon barcodes. A similar process is done for additional barcodes or other sequence features that are of interest.

Shown below are few exemplary definition of sequence models for the HMM/CM.

#=GF ID N1_cdc
N1_cdc
(SEQ ID NO: 119)
ACACTGACGACATGGTTCTACANNNNNNNNNNGACCCCAAAATCAGCGAAATGCACCCCGCATTACGTTTGGTGGACCCTCAG
ATTCAACTGGCAGTAACCAGANNNNNNNNNNAGACCAAGTCTCTGCTACCGTA
#=GC SS_cons
(SEQ ID NO: 120)
ACACTGACGACATGGTTCTACA>>>>>>>>>>GACCCCAAAATCAGCGAAATGCACCCCGCATTACGTTTGGTGGACCCTCAGAT
TCAACTGGCAGTAACCAGA<<<<<<<<<<AGACCAAGTCTCTGCTACCGTA
//
# STOCKHOLM 1.0
#=GF ID N2_cdc
N2_cdc
(SEQ ID NO: 121)
ACACTGACGACATGGTTCTACANNNNNNNNNNTTACAAACATTGGCCGCAAATTGCACAATTTGCCCCCAGCGCTTCAGCGTTC
TTCGGAATGTCGCGCNNNNNNNNNNAGACCAAGTCTCTGCTACCGTA
#=GC SS_cons
(SEQ ID NO: 122)
ACACTGACGACATGGTTCTACA>>>>>>>>>>TTACAAACATTGGCCGCAAATTGCACAATTTGCCCCCAGGGCTTCAGCGTTOTT
CGGAATGTOGCGC<<<<<<<<<<AGACCAAGTCTCTGCTACCGTA
//
# STOCKHOLM 1.0
#=GF ID E-Guelph
E-Guelph
(SEQ ID NO: 123)
ACACTGACGACATGGTTCTACANNNNNNNNNNgtactcattcgtttcggaagagACAGGTACGTTAATAGTTAATAGCGTACTTCTTTTTCT
TGCTTTCGTGGTATTCTTGCTAGTTACACTAGCCATCCTTACTGCGCTTCGATTGTGTGCGTACTGCTGCAATATTGTTAACGTG
AGTCTTGTAAAACCTTCTTTTTACGTTTACTCTCGTGTTAAAAATCTGAATTCTTCTAGAGTTCCTGATCTTCTGGNNNNNNNNNN
AGACCAAGTCTCTGCTACCGTA
#=GC SS_cons
(SEQ ID NO: 124)
ACACTGACGACATGGTTCTACA>>>>>>>>>>gtactcattcgtttcggaagagACAGGTACGTTAATAGTTAATAGCGTACTTCTTTTTCTTG
CTTTCGTGGTATTCTTGCTAGTTACACTAGCCATCCTTACTGCGCTTCGATTGTGTGCGTACTGCTGCAATATTGTTAACGTGAG
TCTTGTAAAACCTTCTTTTTACGTTTACTCTCGTGTTAAAAATCTGAATTCTTCTAGAGTTCCTGATOTTCTGG<<<<<<<<<<AGAC
CAAGTCTCTGCTACCGTA
//
# STOCKHOLM 1.0
#=GF ID N-AMPD
N-HKU
(SEQ ID NO: 125)

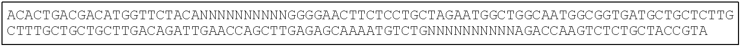

#=GC SS_cons
(SEQ ID NO: 126)

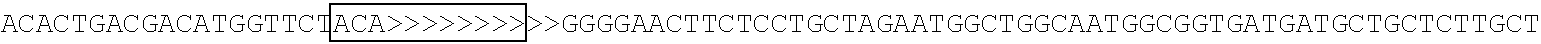

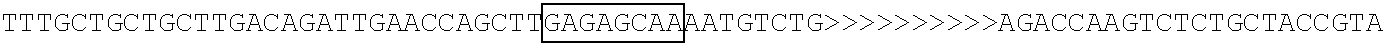

//
# STOCKHOLM 1.0
#-GF ID RNAseP
RNAseP
(SEQ ID NO: 127)
ACACTGACGACATGGTTCTACANNNNNNNNNNAGATTTGGACCTGCGAGCGGGTTCTGACCTGAAGGCTCTGCGCGGACTTGT
GGAGACAGCCGCTCNNNNNNNNNNAGACCAAGTCTCTGCTACCGTA
#=GC SS_cons
(SEQ ID NO: 128)
ACACTGACGACATGGTTCTACA>>>>>>>>>>AGATTTGGACCTGCGAGCGGGTTCTGACCTGAAGGCTCTGCGCGGACTTGTG
GAGACAGCCGCTC<<<<<<<<<<AGACCAAGTCTCTGCTACCGTA
//
# STOCKHOLM 1.0
#=GF ID TM2
TM2
(SEQ ID NO: 129)
ACACTGACGACATGGTTCTACANNNNNNNNNNTGCTCGGGGATACCCGTACCTCGGGTTTCCGTCTTGCTCGTATCGCTCGAG
AACGCAAGTTNNNNNNNNNNAGACCAAGTCTCTGCTACCGTA
#=GC SS_cons
(SEQ ID NO: 130)
ACACTGACGACATGGTTCTACA>>>>>>>>>>TGCTCGCGGATACCCGTACCTCGGGTTTCCGTCTTGCTCGTATCGCTCGAGAA
CGCAAGTT<<<<<<<<<<AGACCAAGTCTCTGCTACCGTA
//
# STOCKHOLM 1.0
#=GF ID TM3
TM3
(SEQ ID NO: 131)
ACACTGACGACATGGTTCTACANNNNNNNNNNGGCTGCTCGCGGATACCCGTACCTCGGGTTTCCGTCTTGCTCGTATOGCTC
GAGAACGCAAGTTCTTCAGCGAAAAGCACGACAGTGGTCGCTACATAGCGTGGTTCCATACTGGAGGTGAAATCACCGACAGC
ATGAAGTCCGCCGGCGTGCGCGTTATACGCACTTCGGAGTGGCTAACGCCGGTTCCCACATTCCCTCANNNNNNNNNNAGAC
CAAGTCTCTGCTACCGTA
#=GC SS_cons
(SEQ ID NO: 132)
ACACTGACGACATGGTTCTACA>>>>>>>>>>GGCTGCTCGCGGATACCCGTACCTCGGGTTTCCGTCTTGOTCGTATCGOTCGA
GAACGCAAGTTCTTCAGCGAAAAGCACGACAGTGGTCGCTACATAGCGTGGTTCCATACTGGAGGTGAAATCACCGAGAGCAT
GAAGTCCGCCGGGGTGCGCGTTATACGCACTTCGGAGTGGCTAACGCCGGTTCCCACATTCCCTCA<<<<<<<<<<AGACCAAG
TCTCTGCTACCGTA

The exemplary file shown is a Stockholm-format file, containing sequence and annotations for exemplary targets N1_cdc. N2_cdc, E-Guelph, N-AMPD, from various regions of SARS-CoV-2 genes N, E. Human gene RNAseP and/or TM3 are used as control. The boxed regions correspond to exemplary annotated spacer or adapter, the barcode location, the viral primers and the template. The ‘>’ signs in the matched consensus “SS_cons” sequences are carried through by the HMM/CM engine and aligned in the output report so that can readily identify bases which are barcode and disregard that are not.

FIGS. 5A and FIG. 5B depict exemplary alignment reports for E-Guelph and RNAseP, respectively, specific regions of SARS-CoV-2 genes N. E, human gene RNAseP. In each of the figures, FIG. 5A and FIG. 5B, there are stacked alignments representing (from top down): the consensus (“model”) sequence, the gene model used, the matches to the gene model, the actual read data from the nanopore sequencer (the lines beginning with “67f21 . . . ” and “46229 . . . ”), and various positions for the model-to-sequence matches (position in model and position in the nanopore read). There are also scores given at the top of each alignment, including scores representing the bitscore, the E-value (statistical significance of a match of this alignment quality relative to the search database of nanopore reads), etc. This report was parsed to generate a comprehensive tally of these data for each of the hundreds of thousands or millions of nanopore reads resulting from a nanopore run, then extract the labelled features (denoted here are two barcodes, namely a single barcode and its reverse complement, but other arbitrary numbers are possible), to store them in a central database. The process of determining a diagnostic read, therefore, is just counting the total number of passing matched alignments and their barcodes for positives and their controls. Negative patients will not have an amplification happen as they lack the pathogen template, so their barcodes will not be present in the amplicon mixture or they may be present at a very low level relative to the actual positives, even in rare cases where template contamination happens in the preparation of the reaction chemistry.

In the exemplary definition of sequence models for the HMM/CM shown above, the category of each amplicon (e.g. N1_cdc, N2_cdc, E-Guelph, N-AMPD, RNAseP, TM3 or an influenza or other virus gene) was determined by selecting the HMM or CM model giving the highest scoring match to the amplicon sequence.

The invariant adapter or spacer sequences are essential because they anchor the alignment of the statistical HMM or CM model to the spacer regions, and so allow for unambiguous annotation of barcode nucleotides in the sequence. The barcodes in the sequence model definition, after all, are listed as “N” or wildcard bases, since their composition is highly variable in nature. The fixed spacers give a region where the aligner can confidently assign a match, and then by process of iterative refinement as the alignment is performed, the barcode regions are identified and annotated. Barcodes should therefore be “internal” to the amplicon by some degree. The adapters/spacers described herein are about 22 bp's in length, and this can be a variable number. It is not preferred to have the barcode be immediately adjacent to the 5′ or 3′ end of the amplicon sequence.

When a two stage barcoding procedure is used, one barcode and the invariant spacer is introduced by the primer used in the amplification process. The second set of barcodes (e.g. the barcodes used to track samples pooled from a stage 1 plate) are ligated to the ends of the amplicon. As a result, the invariant spacer sequence will be located between the two barcodes. This avoids ambiguity that might result from having the two barcodes immediately adjacent to each other. When a two stage barcode procedure is used, the sequence used to generate the HMM or CM model incorporates ambiguity sequences on both sides of the invariant spacer sequence so that the alignment of the sequence read to the statistical model correctly annotates both sets of barcodes. An alternative is to create HMM or CM models for each of the second stage barcodes used and to use the quality of the match to these models to assign the identity of the second stage barcode.

The use of statistical models for representing profiles of multiple sequencing is known to the person of skill in the art. See, e.g., S. R. Eddy, “Profile Hidden Markov Models,” Bioinformatics Review, Vol. 14, no 9, 1998, pages 755-763. The nucleic sequences were analyzed using the HMMER software package, following the user guide which is available from HMMER (Janelia Farm Research Campus, Ashburn, Va.). The output of the HMMER software program is a Profile HMM that characterizes the input sequences. As stated in the user guide, profile HMMs are statistical models of multiple sequence alignments. They capture position-specific information about how conserved each column of the alignment is, and which nucleic residues are most likely to occur at each position. The output of the HMMER software program contains sequence reads with dual barcodes that must pass minimum Leventshein distance score vs reference barcode candidates. The reads are also assigned a per-read alignment score (pre-defined per-gene) with a minimum bitscore filter. Passing reads stored in a central database with full target sequence annotation, model fit, bitscore, barcode locations, barcode distance, and other metrics. In certain embodiments, determining a consensus sequence requires identification of multiple sets of sequential positions (e.g., using different thresholds for different sets) and generating multiple consensus sequences for the multiple sets of sequential positions. The multiple consensus sequences generated can be ranked, e.g., based on probabilities, and given a probabilistic score (e.g., bit score) by converting the probability parameters in a profile HMM to additive log-odds scores before aligning and scoring a query sequence (see, Barrett et al., 1997).

In most embodiments provided herein, the algorithms are computer-implemented methods. In those embodiments, the algorithm and/or results (e.g., consensus barcoded amplicon sequences generated) are stored on computer readable medium, and/or displayed on a screen or on a paper print-out. Full sequence information was stored in PostgreSQL AWS database for passing and failing amplicons. Barcode matches (inner per-patient barcodes and outer per-plate barcodes) were stored to assign reads to original PCR reactions. HMM/CM scores, model fits, and locations in raw FASTA files were saved. Sequence and alignments/matches tables allow cross-reference to LIMS information (plate, batch, etc.). In certain aspects, the results are further analyzed to provide an individual with a diagnosis or prognosis, or to provide a health care professional with information useful for treatment of a disease.

### IV. Method for identifying a target nucleic acid

In one aspect, the present disclosure provides a method for identifying at least one target nucleic acid. The method comprises the steps of obtaining a plurality of biological samples from a plurality of subjects, obtaining total nucleic acid from each of the biological samples, subjecting the plurality of polynucleotides to amplification using an amplification mixture to produce a plurality of amplicons, detecting each of the plurality of amplicons and determining a category of the plurality of amplicons.

In some embodiments, biological samples from a plurality of subjects comprise polynucleotides, i.e., nucleic acids (e.g., DNA or RNA) is obtained from a subject, processed (lysed, amplified, and/or purified) using the methods described herein, and the nucleic acid is sequenced. Nucleic acids can be obtained by methods known in the art. In general, nucleic acids can be extracted from biological samples by a variety of techniques such as those described by manitis et al. molecular cloning: a guide to the Laboratory (Molecular Cloning: A Laboratory Manual), Cold Spring Harbor, N.Y., N. 280-281. (1982), the contents of which are incorporated herein by reference in their entirety.

In some embodiments, biological samples from a plurality of subjects comprise DNA only. In other embodiments, biological samples from a plurality of subjects comprise RNA only. In many embodiments of the method, biological samples from a plurality of subjects comprise a mixture of DNA and RNA. In the embodiments where the biological samples from a plurality of subjects comprise RNA, e.g., mRNA, collected from a subject sample (e.g., a blood sample), an additional processing step of obtaining cDNA reverse-transcribed from the RNA or reverse-transcribing cDNA from the RNA, is required. General methods for DNA/RNA extraction are well known in the art and are disclosed in standard textbooks of Molecular Biology, including Osebia (Ausubel) et al, Current Protocols of Molecular Biology, John Wiley and Sons (1997). Methods for extracting RNA from paraffin-embedded tissues are disclosed, for example, in Rupp (Rupp) and Rocker (Locker), laboratory investments (Labinvest.) 56: A67(1987) and Deanderley (De Andres) et al, BioTechniques 18:42044 (1995). The contents of each of these references are incorporated herein by reference in their entirety. In particular, RNA isolation can be performed using purification kits, buffer sets, and proteases from commercial manufacturers, such as Qiagen, according to the manufacturer's instructions. For example, Qiagen's RNeasy mini-column can be used to isolate all RNA from cells in culture. Other commercially available RNA Isolation kits include the MASTERPURE Complete DNA and RNA purification Kit (MASTERPURE Complete DNA and RNA purification Kit) (EPICENTRE, Madison, Wis.) and the Paraffin Block RNA Isolation Kit (Paraffin Block RNA Isolation Kit) (Ambion. Inc.)). Total RNA can be isolated from tissue samples using RNA Stat-60 (Tel-Test). RNA prepared from the tumor can be isolated, for example, by cesium chloride density gradient centrifugation. Methods and kits for obtaining cDNA reverse-transcribed from the RNA or reverse-transcribing cDNA from the RNA, are well known in the art and are disclosed in, for example, U.S. Pat. No. 5,641,864A, the contents of which are hereby incorporated in its entirety.

In one embodiment, the method comprises obtaining a total RNA from each of the biological samples, reverse transcribing the total RNA from each of the biological samples to obtain a plurality of cDNAs; amplifying the cDNAs using unique sets of forward primers and reverse primers, wherein the primers comprise a set of nucleotides that are complementary to each of the plurality of cDNAs. In another embodiment, the method comprises obtaining a total DNA from each of the biological samples; amplifying the DNAs using unique sets of forward primers and reverse primers, wherein the primers comprise a set of nucleotides that are complementary to each of the plurality of DNAs. In some embodiments, an Ultra-High Throughput PCR Automation is used to amplify the nucleic acid sample (e.g., DNAs and cDNAs) to produce a plurality of amplicons.

In one embodiment of the method, the plurality of polynucleotides are subjected to PCR amplification using an amplification mixture to produce a plurality of amplicons. In one embodiment of the method, the amplification mixture comprises a plurality of primers, the forward primers and the reverse primers. The primers comprise a set of nucleotides that are complementary to each of the polynucleotides that they bind to. In one embodiment, the method described herein provides for the amplification of the cDNAs using an amplification mixture comprising unique sets of forward primers and reverse primers. The primers comprise a set of nucleotides that are complementary to each of the plurality of cDNAs and at least one unique nucleotide barcode sequence. The primer sequence may be from 11 to 35 nucleotides in length, such as from 15 to 25 nucleotides in length. Exemplary primer sequence for use in the methods described herein are provided in SEQ ID NOs.:3-20.

In some embodiments, a single primer can be used amplify all RNA molecules in a sample. For example, the primer can include an RNA complement portion comprised of poly(dT) or random sequence, partially random sequence, and/or nucleotides that can base pair with more than one type of nucleotide. The RNA complement of a cDNA primer will hybridize any RNA sequence to which it is complementary, such as all mRNA (if poly(dT) is used) or all RNA molecules in general (if a generic sequence is used). In this way all of the RNA molecules in a sample can be reverse transcribed. In other embodiments, the primers can include, for example, a cDNA complement portion comprised of random sequence, partially random sequence, and/or nucleotides that can base pair with more than one type of nucleotide.

In some embodiments, a single rolling circle amplification primer can be used to can be used amplify all RNA molecules in a sample. For example, the rolling circle primer can have a random sequence making it complementary to many sequences in the cDNA molecules. In other embodiments, a pair of rolling circle amplification primers can have a complementary portion that is complementary to sequence in the cDNA templates, thus allowing exponential rolling circle amplification with only these two oligonucleotides.

In some embodiments, the plurality of circularized cDNA molecules can be the templates and can then be amplified via rolling circle amplification. Rolling circle amplification can be primed by primer set, each of which are complementary to at least one circularized cDNA template. In some instances, the complementary portion of the primers can be complementary to cDNA sequence. In such instances, the rolling circle amplification primers can be specific for one or a few cDNA templates. Rolling circle amplification primers can have random sequences.

In one embodiment, the method comprises PCR amplification of the target nucleic acid templates to obtain a plurality of amplicons. In the methods described herein, the target nucleic acid templates are also referred to as the target amplified region. The method comprises amplifying the target cDNA templates to obtain a plurality of amplicons. In some embodiments, the method further comprises separating the unique sets of forward primers and reverse primers that have not been extended (i.e., the “unused” primers) from the plurality of amplicons. A nucleic acid sample that contains target nucleic acids to be amplified/extended may be prepared by methods know to a person of skill in the art from any samples that contain nucleic acids of interest. In addition, many kits for nucleic acid preparation are commercially available and may be used, including QIAamp DNA mini kit, QIAamp FFPE Tissue kit, and PAXgene DNA kit. Exemplary samples include, but are not limited to, samples from a human including blood, swabs, body fluid, or materials and fractions obtained from the samples described above, or any cells. In some embodiments, the sample is selected from the group consisting of blood, mucus, saliva, sweat, tears, fluids accumulating in a bodily cavity, urine, ejaculate, vaginal secretion, cerebrospinal fluid, lymph, feces, sputum, decomposition fluid, vomit, sweat, breast milk, serum, and plasma. In specific embodiments, the sample is saliva.

Target nucleic acids are those known to be involved and/or indicative of an infection, disease or disorder. The target nucleic acids or a target amplified region described herein can be obtained from a sample comprising one or more pathogens including, but not limited to, a RNA virus, a DNA virus, a fungus and a bacterium. The infection, disease or disorder may include, but not limited to, various viral infection, bacterial infection and disease caused by other pathogens, target nucleic acid is obtained from a sample comprising one or more pathogens selected from the group consisting of a RNA virus, a DNA virus, a fungus and a bacterium.

In some embodiments of the methods described herein, the target nucleic acid is obtained from a sample comprising one or more pathogens selected from a non-limiting group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus. Cytomegalovirus, Dengue virus, Dracunculus medinensis, Eastern Equine Encephalitis virus, Ebola virus, Echinococcus granulosus, Echinococcus multilocularis, Enterobacter cloacae. Enterococcus faecium, Enteroviruses, Epstein-Bar virus, Escherichia coli, Fasciola giganta, Fasciola hepatica. Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2. Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus. Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni. Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae. Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above, or is another potentially novel or uncharacterized pathogen sharing distinctive nucleic acid sequences with a pathogen in the aforementioned group. In one embodiment, the target nucleic acid is obtained from a salivary sample comprising SARS-CoV-2.

In many embodiments of the method, at least one unique barcode sequence and its reverse complement is introduced into each of the forward and reverse primers, respectively, uniquely identifying each amplicon after amplification. The forward and/or reverse primer comprises a unique nucleotide sequence referred to as the barcode sequence. This sequence will uniquely identify a particular target nucleic acid. The length of the barcode sequence may be from 3 to 20 nucleotides, such as from 5 to 15 nucleotides in length. Non-limiting exemplary barcode sequences for use in the methods described herein are provided in Table 1. As described herein, the exemplary sequences in Table 1 are selected from within the 3000+ total barcodes that are maximally Levenshtein-distance separated. In some embodiments, 384 maximally Levenshtein-distance separated barcode sequences are selected. The selection of barcode sequences is done algorithmically and yields different results depending on the selection size. In may embodiments of the methods and compositions provided herein, the number of barcode sequences selected is based on the size of the barcode pool that the primers are assembled from.

In some embodiments, the barcode sequence may be completely random, that is, any one of A. T, G, and C may be at any position of the barcode sequence. Random barcodes are economical to synthesize. In other embodiments, the barcode sequence is synthetically individually synthesized (e.g., Twist Bio) which ensures different barcode oligos, but each synthesized independently. In the embodiments described herein, the barcodes are part of the forward and reverse primer sequences. Exemplary barcoded primer sequences are provided in SEQ. ID. Nos 1 and 2. For example, a set of barcoded matching forward and reverse primers generates a barcoded amplicon with the same (or a forward/reverse complemented) barcode on both 5′ and 3′ ends of the primed viral sequence. The viral sequence includes the primers themselves. In certain embodiments, the barcode sequences are semi-defined or completely defined. Using such sequences can mitigate barcode errors. However, doing so, especially using completely defined barcode sequences for many different primers in high multiplex PCR, may be cost prohibitive in some cases.

In some embodiments of the method, first unique barcode and its reverse complement and the first pair of adapter sequences, also referred to herein as the inner barcodes, are introduced by the primers (also referred herein as barcoded primers) used in the amplification process. In some embodiments of the method, second unique barcode sequence and its reverse complement, also referred to herein as the outer barcodes (e.g., plate/batch identifiers), are added to the barcoded amplicons, typically using a ligation reaction. Ligated outer barcodes avoids cross-amplification inherent to 2nd PCR stage-based amplifications. The ligation (using a DNA ligase enzyme) step appends a second set of DNA fragments containing “outer” barcodes on both ends of the first barcoded amplicons. The ligation allows for combinatorial assembly of barcodes and allows for massive multiplexing (e.g. 384 inner barcodes×384 outer barcodes=147456 unique dual-barcoded amplicons.) The inner barcode, in some instances, is a patient or well specific barcode to annotate a specific sample from a plurality of distinct samples in a plate with at least 96 wells. The outer barcode can denote a specific batch or can be a plate identifier when there is a plurality of distinct samples in distinct plates with multiple batches of plates. Barcode sequences and primers are selected from a very large, validated IDT barcode library that has been screened for secondary structure interactions, resulting in a highly optimized, error tolerant barcode design.

Extension of barcoded primers may be performed by combining all primers, and target nucleic acids in a nucleic acid sample with a DNA polymerase in reaction buffer. Preferably, annealing to target nucleic acids by barcoded primers and/or extension of barcoded primers is performed at an elevated temperature, for example, at 50° C. to 75° C., such as at 55° C., 60° C., 65° C. 70° C. or 72° C., to increase the annealing specificity between target nucleic acids and barcoded primers. The target nucleic acids in the nucleic acid sample are typically first denatured, such as by incubated at a high temperature (e.g., 95° C. or 98° C.), before annealing with barcoded primers. Target nucleic acid denaturing, primer annealing, and primer extension may be performed in a thermal cycler.

In certain embodiments wherein a hot-start DNA polymerase is used, DNA polymerase activation may also be simultaneously performed with target nucleic acid denaturing in a thermal cycler. Preferably, DNA polymerases used for barcoded primer extension are thermostable. Exemplary DNA polymerases include Taq polymerase (from Thermus aquaticus), Tfi polymerase (from Thermus filiformis), Bst polymerase (from Bacillus stearothermophilus), Pfu polymerase (from Pyrococcus furiosus), Tth polymerase (from Thermus thermophilus), Pow polymerase (from Pyrococcus woesei), Tli polymerase (from Thermococcus litoralis), Ultima polymerase (from Thermotoga maritima), KOD polymerase (from KOD Hot Start polymerase (EMD Biosciences), Deep Vent™ DNA polymerase (New England Biolabs), Platinum® Taq DNA Polymerase High Fidelity (Invitrogen).

In some embodiments of the method, the forward and reverse primers include one or more pairs of adapter sequences. In other embodiments, the adapter sequences are ligated to the barcode sequences that are on both 5′ and 3′ ends of the primed target amplified region sequence. In many of the embodiments described herein, the adapter sequence provides a function of a spacer sequence. In some instances, the adapter sequence acts as a marker during sequence reads to signal the end of a barcode sequence and/or the beginning of the next barcode sequence. In some embodiments described herein, the adapter sequence may comprise a universal sequence. In specific embodiment, the adapter sequence is a conserved sequence. In some embodiments of the method, the adapter sequence comprises at least 10 nucleotides. In some other embodiments of the method, the adapter sequence comprises between 10 to 15 nucleotides. In one embodiment of the method, the adapter sequence comprises 10 nucleotides. Non-limiting exemplary adapter sequences are provided in SEQ ID Nos 21 and 22. In some embodiments of the methods described herein, a single stage barcoding with a first unique barcode sequence and its reverse complement is used. In such embodiments, first unique barcode and its reverse complement and the first pair of adapter sequences are introduced by the primers used in the amplification process. In other embodiments of the method described herein, a two stage barcoding with a first unique barcode sequence and its reverse complement and a second unique barcode sequence and its reverse complement, are used. In these embodiments, one barcode (e.g., first unique barcode sequence) and the adapter sequence (e.g., first pair of adapter sequences) is introduced by the primer used in the amplification process. The second set of barcodes (e.g. the barcodes used to track samples pooled from a stage 1 plate, second unique barcode sequence) are ligated to the ends of the amplicon. As a result, the invariant adapter sequence will be located between the two barcodes. Other adapter sequences can be generated and fall within the scope of this disclosure.

The universal primer sequence of a primer is a sequence that may be used for further amplification. A number of different amplification strategies are known to a person of skill in the art. All amplification technologies rely on a primer for initiation and this primer could be engineered to incorporate a barcode. Preferably, this sequence does not have significant homology (i.e., has less than 50% sequence identity over its full length) to target nucleic acids of interest or other nucleic acids in a nucleic acid sample. As described above, a plurality of primers is used to assign different barcodes to different target nucleic acids. In some embodiments, the target nucleic acids are from a single pathogen while in other embodiments, the target nucleic acids are from at least two different pathogens. Among the plurality of primers, the universal primer sequences can be the same, but the target-specific sequences of the primers (i.e., sequences complementary to the target nucleotide sequences) are different. The same universal sequence in sequence of different primers allows subsequent amplification of the amplicon using a single primer.

In some embodiments of the method described herein, a 5′ adaptor region sequence and/or a sample identification region (e.g., unique barcode nucleotide sequence) are added to all cDNAs from a single sample, e.g., during reverse transcription. In some aspects, 3′ specific primers can be used to amplify any polynucleotide in the single sample. In some aspects, polynucleotides are amplified that have a 5′ variable region, e.g., single stranded RNAs from viral particles without needing multiple degenerate 5′ primers to amplify a specific region of interest. Primers can also be specific for IgG, IgM, IgD, IgA, IgE, TCR chains, and other genes of interest.

In some embodiments, an adapter region includes 2, 3, 4, 5, 6, 7, 8, 9, 10 or more G's. In some aspects, a cDNA includes 1, 2, 3, 4, 5, 6, 7, 8, 9, 10 or more C's on its 3′ end. In some embodiments, adapter regions are attached to the 5′ ends of cDNAs. In other embodiments, adapter regions are attached to the 3 ends of cDNAs. In yet another embodiment, adapter regions are attached to the 5′ and 3′ ends of cDNAs. Different methods to attach adaptor regions exist, including but not limited to, doing PCR with primers with 5′ flanking adaptor region sequences, sticky and blunt end ligations, template-switching-mediated addition of nucleotides, or other methods to covalently attach nucleotides to the 5′ end, to the 3′ end, or to the 5′ and 3′ ends of the polynucleotides. These methods can employ properties of enzymes commonly used in molecular biology. PCR can use, e.g., thermophilic DNA polymerase. Sticky ends that are complementary or substantially complementary are created through either cutting dsDNA with restriction enzymes that leave overhanging ends or through 3′ tailing activities of enzymes such as TdT (terminal transferase). Sticky and blunt ends can then be ligated with a complementary adaptor region using ligases such as T4 ligase. Methods for ligating adapters to blunt-ended nucleic acids are known in the art and may be used in generating sequencing libraries from amplification products of PCR as provided herein. Exemplary methods include those described in Sambrook J and Russell D W, editors, (2001) Molecular Cloning: A Laboratory Manual. 3rd ed. Cold Spring Harbor, NY: Cold Spring Harbor Laboratory, QIAGEN GENEREAD™ Library Prep (L) Handbook and U.S. Patent Application Publication Nos. 201 0/01 97509, 201 3/0005613. In one embodiment, the method described herein optionally provides for the amplification of the cDNAs using a plurality of amplification primer.

In one embodiment of the method described herein, the number of unique barcoded primers is at least 50, at least 100, at least 300, at least 500, at least 750, or at least 1000. The use of such unique barcoded primers in a single reaction allow analysis of a relatively large number of target nucleic acids, such as parallel sequencing analysis of polynucleotides from multiple samples. For an individual target nucleic acid or amplicon, whether the barcoded primer anneals to the plus or minus stand of DNA can be randomly selected. For example, when multiplexing different viral targets from the same individual, and the multiplexing could be as few as 2 or as many as 1000.

In one embodiment, the method described herein, optionally includes a step to separate unused primers (i.e., barcoded primers that have not been extended) from amplicons. The removal of unused primers minimizes the risk of the “barcode resampling” problem, that is, the same DNA template being associated with multiple molecular barcodes. Such a problem would defeat the benefits of molecular barcoding. Separation of unused primers may be performed by size selection purification. The amplicons may be purified from unextended primers using either bead or silica column based size selection system, such as Agencourt AMPure XP system and GeneRead Size Selection system. If needed, two or more rounds of purification with such a system may be used. Alternatively, a single-stranded DNA cleanup step by an exonuclease enzyme (e.g. ExoSAP-IT™ from ThermoFisher), can be incorporated into the method described herein. One additional way of avoiding the problem of “barcode resampling” is to not perform two PCR steps, but perform a PCR step to make the first primers, and then ligate a second outer barcode (without amplification). In one embodiment, the method described herein may further comprise an additional amplification of the amplicons. The additional amplification may be performed in the presence of a pair of universal primers described above.

The methods described herein comprises a detection step for each of the plurality of amplicons. In many embodiments, the detection is performed by reading sequences of the unique barcodes in each of the amplicon. In some embodiments of the method, sequencing at least one positive control sample, where the positive control sample comprises the target nucleic acid. In the embodiments of the method described herein, a high throughput sequencing is used to detect the unique barcodes in the amplicons. Any high throughput sequencing platforms known in the art may be used to sequence the sequencing libraries prepared as described herein (see, Myllykangas et al., Bioinformatics for High Throughput Sequencing, Rodriguez-Ezpeleta et al. (eds.), Springer Science+Business Media, LLC, 2012, pages 11-25). Exemplary high throughput DNA sequencing systems include, but are not limited to, the Oxford Nanpore platform, including MinION and PromethION instruments, the GS FLX sequencing system originally developed by 454 Life Sciences and later acquired by Roche (Basel, Switzerland), Genome Analyzer developed by Solexa and later acquired by Illumina Inc. (San Diego, CA) (see, Bentley, Curr Opin Genet Dev 16:545-52, 2006: Bentley et al., Nature 456:53-59, 2008), the SOLiD sequence system by Life Technologies (Foster City, CA) (see. Smith et al., Nucleic Acid Res 38: e 142, 2010; Valouev et al., Genome Res 18:1051-63, 2008), CGA developed by Complete Genomics and acquired by BGI (see, Drmanac et al., Science 327:78-81, 2010), PacBio RS sequencing technology developed by Pacific Biosciences (Menlo Park, CA) (see, Eid et al., Science 323: 133-8, 2009), and Ion Torrent developed by Life Technologies Corporation (see, U.S. Patent Application Publication Nos. 2009/0026082; 2010/01 37143; and 2010/0282617). The Oxford Nanopore DNA sequencing systems used in the methods described herein are more suited to rapidly and accurately read amplicons that are routinely over 250 bp in length. The Illumina sequencing system may not be as suited to the methods described herein compared to the Oxford Nanopore DNA sequencing systems due to long processing time and sequencing-by-synthesis, yielding relatively short reads.

In some embodiments of the method, detecting comprises sequencing each of the plurality of amplicons comprising the pair of adapter sequences and the first unique barcode sequence and its reverse complement. In other embodiments of the method, detecting comprises sequencing each of the plurality of amplicons comprising the pair of adapter sequences, the first unique barcode sequence and its reverse complement, and the second unique barcode sequence and its reverse complement. In many embodiments, detecting is performed by reading a sequencing data file with a software program. The sequencing data file is in a FASTA/FASTQ format or a is a Stockholm-format file.

In some embodiments, the method identifies one target nucleic acid. In some embodiments, the method identifies two or more target nucleic acids from the same pathogen. In some embodiments, the method identifies two or more target nucleic acids from the two different pathogen s of the same type (e.g., viral pathogens). In some embodiments, the method identifies two or more target nucleic acids from the two different pathogen s different types (e.g., a viral and a bacterial pathogen). In many embodiments, the method comprises a step of determining a category of the plurality of amplicons. A key step in the methods described herein is the sequence analysis of the amplicon insert. In many embodiments of the method, for a given sample, identical barcodes are used for the positive control and for each of the plurality of the target nucleic acids of interest that are being tested for. When the amplicons are counted, it is the sequence of the insert (e.g., target amplified region) that determines how to categorize and count the amplicon. For example, if the target amplified region sequence is present in the amplicon, then the amplicon is categorized as a hit and counted. If the target amplified region sequence is not present in an amplicon, it may be categorized and counted as a control. Thus, the sequence of the insert (e.g., target amplified region) is also how the sequence variants of the pathogenic determinants are recognized and novel variants are discovered without having prior knowledge of their existence. In many embodiments of the methods described herein, determining the category of each the plurality of amplicons comprising the polynucleotides from the target amplified region indicates that the corresponding subject has the target nucleic acid.

In some embodiments, the methods described herein are applied to a plurality of distinct samples in a plate with at least 96 wells, at least 384 wells, at least 1536 wells, or more wells. In further aspects, the methods described herein are applied to distinct samples in at least one, two, three, four, five, six, seven, eight, ten, fifteen, twenty, thirty, three hundred and eighty-four or more plates with at least 96 wells each. In other aspects, the methods described herein are applied to distinct samples in at least one, two, three, four, five, six, seven, eight, ten, fifteen, twenty, thirty, three hundred and eighty-four or more plates with at least 384 wells each.

### V. Methods for Detecting Sequence Variants in a Nucleic Acid Sample

The methods described herein can detect one or more sequence variants in a nucleic acid sample. A sequence variant can be any variation with respect to a reference sequence (e.g., a nucleic acid sample from a healthy human or even a nucleic acid sample from a patient suspected of having a SARS-Cov-2 infection.) A sequence variation may consist of a mutation, insertion of, or deletion of a single nucleotide, or of a plurality of nucleotides (e.g. 2, 3, 4, 5, 6, 7, 8, 9, 10, or more nucleotides). Where a sequence variant comprises two or more nucleotide differences, the nucleotides that are different may be contiguous with one another, or discontinuous. Non-limiting examples of types of sequence variants include random mutations occurring in a genome, single nucleotide polymorphisms (SNP), deletion/insertion polymorphisms (DIP), retrotransposon-based insertion polymorphisms, and sequence specific amplified polymorphism. The methods used herein can detect any sequence variants. For example, a disclosure for detecting point mutations in a polynucleotide sequence can also be applicable to the detection of indels or deletions.

The methods provided herein are used to detect sequence variants from nucleic acid sample obtained from a biological sample. In some embodiments, the resulting information can be used to identify mutations present in nucleic acid sample obtained from the subject.

Polynucleotides from a sample may be any of a variety of polynucleotides, including but not limited to, DNA, RNA, ribosomal RNA (rRNA), transfer RNA (tRNA), micro RNA (miRNA), messenger RNA (mRNA), fragments of any of these, or combinations of any two or more of these. In some embodiments, samples comprise DNA. In some embodiments, samples comprise genomic DNA. In some embodiments, samples comprise plasmid DNA, bacterial artificial chromosomes, oligonucleotide tags, or combinations thereof. In some embodiments, the samples comprise DNA generated by amplification, such as by primer extension reactions using any suitable combination of primers and a DNA polymerase, including but not limited to polymerase chain reaction (PCR), reverse transcription, and combinations thereof. In some embodiments, samples comprise RNA. In some embodiments, the sample can comprise RNA, e.g., mRNA, collected from a subject sample (e.g., a blood sample). General methods for RNA extraction are well known in the art. In particular, RNA isolation can be performed using purification kits, buffer sets, and proteases from commercial manufacturers, such as Qiagen, according to the manufacturer's instructions. Where the template for the primer extension reaction is RNA, the product of reverse transcription is referred to as complementary DNA (cDNA). In some embodiments, samples comprise a mixture of DNA and RNA. In the instances of the samples comprising a mixture of DNA and RNA (e.g. in coinfection), the reverse transcriptase (RT) is added in and is inactive for the DNA molecules and reverse transcribes in the RNA molecules. In some embodiments, a sample, i.e., nucleic acid (e.g.. DNA or RNA) is obtained from a subject, processed (lysed, amplified, and/or purified) using the methods described herein, and the nucleic acid is sequenced.

One aspect of the disclosure is directed to a method for detecting sequence variants in a nucleic acid sample. The first step involves performing an amplification reaction with the sample of nucleic acid with an amplification mixture to produce a plurality of amplicons. The sample of nucleic acid comprises a plurality of polynucleotides obtained from a plurality of subjects suspected of having a target nucleic acid that is a determinant of an infection. In many embodiments described herein, the target nucleic acid is contained within a genomic region of the pathogen that is referred to herein as a target amplification region. In the embodiments described herein, the amplification mixture comprises a plurality of primers, at least one unique barcode sequence (e.g., a first unique barcode sequence and its reverse complement), and at least one pair of adapter sequences. In some embodiments, each of the plurality of the primers comprise a set of nucleotides that are complementary to the nucleotides in the target amplification region. The unique barcode sequence identifies the biological sample obtained from the specific subject. The pair of adapter sequences, in many instances, block the primers to allow addition of a second unique barcode sequence to each of the plurality of amplicons. In some embodiments, the sample of nucleic acid comprises RNA molecules, and the first step further comprises obtaining cDNA reverse-transcribed from the RNA or reverse-transcribing cDNA from the RNA before performing the amplification reaction.

The second step of the method to detect sequence variations comprises detecting, and optionally quantitating, the plurality of amplicons. In many embodiments of the method, the detecting step comprises determining a nucleic acid sequence in parallel of substantially identical copies of the plurality of amplicons on a single instrument. In the embodiments of the method described herein, a high throughput sequencing is used to detect the unique barcodes in the amplicons. Any high throughput sequencing platforms known in the art may be used to sequence the sequencing libraries prepared as described herein. Exemplary high throughput sequencing systems include, but are not limited to, the Oxford Nanpore platform, including MinION and PromethION instruments, the GS FLX sequencing system originally developed by 454 Life Sciences and later acquired by Roche (Basel. Switzerland), Genome Analyzer developed by Solexa and later acquired by Illumina Inc. (San Diego, CA), the SOLiD sequence system by Life Technologies (Foster City, CA), CGA developed by Complete Genomics and acquired by BGI. PacBio RS sequencing technology developed by Pacific Biosciences (Menlo Park, CA), and Ion Torrent developed by Life Technologies Corporation. The Oxford Nanopore DNA sequencing systems used in the methods described herein are more suited to rapidly and accurately read amplicons that are routinely over 250 bp in length.

The third step of the method comprises a step of determining a category of the plurality of amplicons. As described earlier, this is a key step that is directed to the sequence analysis of the amplicon insert. When the amplicons are counted, it is the sequence of the insert (e.g., target amplified region) that determines how to categorize and count the amplicon. The sequence of the insert (e.g., target amplified region) is how the sequence variants of the pathogenic determinants are recognized and novel variants are discovered without having prior knowledge of their existence. In many embodiments of the methods described herein, determining the category of each the plurality of amplicons comprising the polynucleotides from the target amplified region indicates that the corresponding subject has a particular variant of the target nucleic acid.

The fourth step of the method is directed to the detection of sequence variations. The sequence variations are detected in the methods described herein by a sequencing reaction performed simultaneously on the plurality amplicons to determine a plurality of nucleic acid sequences corresponding to sequence variants (e.g., point mutations in a target amplified region corresponding to a viral genome). Various methods of sequencing and algorithms using the sequencing data to perform multiple sequence alignment, are known in the art and are described herein. Any high throughput sequencing platforms known in the art may be used to sequence the sequencing libraries prepared as described herein. Exemplary high throughput DNA sequencing systems include, but are not limited to, the Oxford Nanpore platform, including MinION and PromethION instruments, the GS FLX sequencing system, Genome Analyzer, the SOLiD sequence system, CGA, PacBio RS sequencing technology and Ion Torrent. The Oxford Nanopore DNA sequencing systems (e.g., ONT MinION or GridION) used in the methods described herein are more suited to rapidly and accurately read amplicons that are routinely over 250 bp in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, for example, by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT. FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). In some embodiments of the method, the PCR amplicons from pooled library preparations were sequenced on ONT MinION or GridION to obtain raw ONT FAST5 sequencing output files. In these embodiments, the output files were subjected to high-accuracy ONT GPU-based base caller to yield raw FASTA/FASTQ or Stockholm-format files. The raw files were run on the HMMER3 and CM sequence alignment and annotation engines. The HMM/CM engines apply the statistical pattern classification algorithm to generate the consensus sequence by a) maximizing a likelihood based upon the replicate sequence reads, and/or b) using a context dependent alignment model parameter based upon a whole genome multiple sequence alignment.

A non-limiting exemplary workflow for determining sequence variations in samples obtained from patients suspected of having SARS-CoV-2 begins with the sequencer reading individual non-ligated amplicons. In many instances, 100-10⁵ depth coverage from positive sample are obtained. The HMM or CM statistical models are used to segment these reads into their constituent amplicon sequences which are individually analyzed. The HMM aligns and annotates amplicon features. Then the patient/batch ID's are obtained by demultiplexing barcode region. Multiple sequence alignments are performed using HMM or CM software on the intervening region to yield high-accuracy consensus sequence. The sequence alignments are then mapped to Genbank/GISAID SARS-CoV-2 reference. The alignments compare pre-defined S/RBD protein reference residues of interest to sequences from the samples to record novel variant residues. The record of novel variants can be submitted to a centralized variant surveillance database and/or provided with the final report of each patient with annotation of antibody/vaccine evasion risk.

Following sequencing, the frequency at which the sequence variants occur, may also be determined by analyzing the sequences from the plurality of nucleic acid samples obtained from different subject population. As an example, if 100000 sequences are determined and 99000 sequences read “gau” while 1000 sequences read “gcu,” the “gau” sequence encoding for an aspartate may be said to have a frequency of 90% while the “gcu” variant encoding for an alanine in that position would have a frequency of 10%. In some embodiments, the methods described herein may detect sequence variations which occurs in less than 10%, less than 5%, or less than 2% of the sequences read. In other embodiments, the method may detect sequence variations which occurs in less than 1%, such as less than 0.5% or less than 0.2% of the sequences read. Typical ranges of detection sensitivity may be between 0.1% and 100%, between 0.1% and 50%, between 0.1% and 10% such as between 0.2% and 5%.

One advantage of the PCR based method described herein is that no a priori knowledge of variation is required for the method. Because the method is based on nucleic acid sequencing, all variation in one location that is amplifies using primers, would be detected. Furthermore, no cloning is required for the sequencing. A nucleic acid sample is amplified and sequenced in a series of steps without the need for cloning, subcloning, and culturing of the cloned nucleic acid. The aspects described above for detection of sequence variations are particularly useful. For example, in one embodiment, the methods described herein can detect various mutant SARS-CoV-2 strains in patient samples. Non limiting examples of the mutant SARS-CoV-2 strains that can be detected by the methods described herein include SARS-CoV-2 variants carrying T95I, D253G, L452Rm E484K, S477N. N501Y 1D614G and A701V point mutations in polynucleotide encoding a spike protein, a receptor binding domain and/or a nucleocapsid protein. In some embodiments method, the nucleic acid sample may be derived from an SARS-CoV-2 RNA source (e.g. a human patient infected with SARS-CoV-2) comprising a detectable titer of virus. In typical embodiments of the method described herein, the source may include a sample from a human subject that includes collected tissue or fluid samples from an SARS-CoV-2 infected patient that may or may not have been exposed to a drug/plasma/vaccine treatment regimen (i.e. the patient may or may not be “drug naïve”). The variations may be correlated with the severity of the disease symptoms, increased mortality, increased spread and/or known resistance or newly identified resistance to treatment modalities. The methods described herein also provide a measure of frequency of each of the variants in a sample population that can be employed to determine the effectiveness of the vaccination programs or alter a therapeutic regimen that may include avoidance of one or more drugs, drug classes, or drug combinations that will have little therapeutic benefit.

Other applications of the described methods include population studies of sequence variants, nucleic samples may be collected from a population of organisms and combined and analyzed in one experiment to determine sequence variation frequencies in a particular region of a viral genome. The populations of organisms may include, for example, a population of humans, a population of livestock, and the like. These population studies can indicate “hot spots” for mutations in a viral genome and such information can be valuable in the design of drugs and/or vaccines.

### VI. Multiplex of Arrays and Use Thereof in Identifying Infections

In one aspect, the disclosure provides a multiplex of array for detecting at least one target protein from multiple samples. The multiplex array comprises a plurality of capture agents bound to a plurality of uniquely labeled beads. Each uniquely labeled bead comprises a plurality of a unique capture agent, at least one first oligonucleotide sequence that is designed to be bound to at least one bead, at least one secondary antibody conjugated with a second oligonucleotide sequence and at least one unique nucleotide barcode sequence in the circular amplicon. In many embodiments of the array described herein, the bead is coated with an antigen that specifically binds at least one target protein. In some embodiments of the array described herein the second oligonucleotide sequence is designed to be amplified to form a circular amplicon when the second oligonucleotide sequence is in close proximity to the first oligonucleotide sequence. In some embodiments, the first oligonucleotide sequence, or the second oligonucleotide sequence, or both, comprise at least one unique barcode sequence. In some embodiments, the first oligonucleotide sequence is covalently bound to a polypeptide coated on the bead. In some embodiments, the multiplex of arrays comprise the first oligonucleotide sequence that is covalently bound to an antibody or an antibody fragment, where the antibody or the antibody fragment bind to a polypeptide coated on the bead.

In some embodiments, the multiplex of arrays comprise at least 96 different barcode sequences in the first oligonucleotide sequence, or the second oligonucleotide sequence, or in combination thereof. In some other embodiments, the multiplex of arrays comprise comprises at least 384 different barcode sequences in the first oligonucleotide sequence, or the second oligonucleotide sequence, or in combination thereof. In some embodiments, the multiplex of arrays comprise at least 96 different barcode sequences in the circular amplicon.

Also provided are systems that find use in practicing the subject methods, as described above. For example, in some embodiments, systems for practicing the subject methods may include at least one set or proximity probes; a least one pair of asymmetric connectors; and a nucleic acid ligase. Furthermore, additional reagents that are required or desired in the protocol to be practiced with the system components may be present, which additional reagents include, but are not limited to: pairs of supplementary nucleic acids, single strand binding proteins, and PCR amplification reagents (e.g., nucleotides, buffers, cations, etc.). NGS sequencing reagents, and the like.

In one aspect, the present disclosure provides a method for at least one infection in a plurality of biological samples. The method comprises the first step of incubating a plurality of biological samples with a plurality of beads in the multiplex of array described herein under conditions sufficient for at least one target protein to bind to the unique capture agent of at least one of the beads. In the second step of the method. The beads are washed to remove any proteins that do not bind to the unique capture agents. The next step involves incubating the beads with a plurality of secondary antibodies under conditions where each of the plurality of the secondary antibodies forms a complex with at least one target protein, such that plurality of complexes corresponding to the number of the secondary antibodies bound to the plurality of target proteins, are formed. In the next step, the beads are washed again to remove any secondary antibodies that do not form the complex. In the sixth step, the plurality of complexes are incubated under conditions to allow hybridization of each of the second oligonucleotide sequence to each of the first oligonucleotide sequence such that they form a circular amplicon, such that plurality of amplicons are generated corresponding to the number of the plurality of complexes. The seventh step of the method involves subjecting the plurality of circular amplicons to amplification. In the eighth step, the beads are pooled in the array and the plurality of amplicons are simultaneously detected by high throughput sequencing of the unique barcoded amplicons. In the final step, the category of the plurality of amplicons is determined. As described earlier, determining the category of each the plurality of amplicons comprising the polynucleotides from the target amplified region indicates infection in the corresponding biological sample.

In some embodiments, the method described herein is used for the identification of pathogenic determinants (e.g., bacterial and/or viral infections) in one or more samples. In other embodiments, the method simultaneously detects target proteins such as IgG and IgM immunoglobulins that are indicative of one or more pathogenic infections. The antibody or the antibody fragment detected by the method described herein bind specifically to one or more antigens from pathogens including Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus. Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis. Enterobacter cloacae, Enterococcus faecium, Enteroviruses, Epstein-Barr virus, Escherichia coli. Fasciola giganta, Fasciola hepatica. Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii. Human herpesviruses HHV6. Human herpesviruses HHV7, Human herpesviruses HHV8, Human herpesviruses HSV1, Human herpesviruses HSV2. Human immunodeficiency virus, Human papillomavirus, Influenza virus A. Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica. Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae. Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above, or is another potentially novel or uncharacterized pathogen sharing distinctive nucleic acid sequences with a pathogen in the aforementioned group.

In some embodiments, the method described herein is used for the identification infection caused by one or more RNA viruses in one or more samples. In a specific embodiment, the method described herein is used for identification of a viral infection (e.g., SARS-CoV-2 infection) in one or more biological sample(s) obtained from one or more patients. SARS-CoV-2 is clinically difficult to diagnose and to distinguish. A rapid, reliable and a massively parallel diagnosis is required in suspected cases of SARS-CoV-2 infection. The present disclosure provides such an assay. The assay is based, at least in part, on the discovery that an SARS-CoV-2 viral polynucleotide can be detected (e.g., sequenced) in a one-step or two-step real-time reverse transcription amplification assay for an SARS-CoV-2 viral polynucleotide using unique barcode sequences as sample source identifiers. The assay provided herein can detect antibody or the antibody fragment detected by the method described herein bind specifically to one or more SARS-CoV-2 antigens selected from the group consisting of a spike protein (S), a receptor-binding domain (RBD), a S1 protein, a S2 protein, E gene, S gene, Orflab gene, N-terminal Spike protein domain, a whole protein (S1+S2), and a nucleocapsid (N) protein. The methods provided herein allows for simultaneous detection of SARS-CoV-2 viral polynucleotides from multiple samples obtained from one or more patients having or suspected of having SARS-CoV-2 infection.

In some embodiments, the method described herein is used for the identification of pathogens of important veterinary diseases (e.g. bovine diarrhea, Johne's disease, pig influenza, etc.) The methods described herein can individually detect infected animals within a herd, as long as the animals are labelled to each sample and barcode-primed appropriately).

In some embodiments, the method described herein is used for the identification of one or more target nucleic acids in one or more samples. In some other embodiments, the method described herein is used for the identification of two or more target nucleic acids in one sample. In particular embodiments, the two or more target nucleic acids are pathogenic determinants, or encode for pathogenic determinants, of a single pathogen. In specific embodiments, the two or more target nucleic acids are pathogenic determinants, or encode for pathogenic determinants, of a single RNA virus (e.g., SARS-CoV-2). In other embodiments, the two or more target nucleic acids are pathogenic determinants, or encode for pathogenic determinants, of two or more RNA viruses (e.g., SARS-CoV-2 and Influenza A virus). In another embodiment, the two or more target nucleic acids are pathogenic determinants, or encode for pathogenic determinants, of one or more RNA viruses (e.g., SARS-CoV-2, Influenza A virus) and one or more bacterial pathogens (e.g., Mycobacterium, Streptococcus, Pseudomonas, Shigella, Campylobacter, Chlamydia and Salmonella). Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

### VI. Systems and Method for Identifying a Target Protein-Serology Assay

The serology assay described herein, is a proximity ligation assay (PLA), for detecting an analyte in a sample. This assay combines the principle of “proximity probing” with “molecular barcoding” and multiplex amplification to facilitate massively parallel analysis of the presence of one or more analytes in a plurality of biological samples. The PLA is an assay wherein an analyte is detected by the coincident binding of multiple (i.e. two or more, generally two, three or four) probes, which when brought into proximity by binding to the analyte form a detectable, preferably amplifiable, nucleic acid detection product (e.g., a circular amplicon) by means of which said analyte may be detected. The nucleic acid detection product (e.g., a circular amplicon) can be detected and sequenced by methods known to a person of skill in the art. In the assay described herein, the proximity probes comprise a nucleic acid domain (or moiety) linked to the analyte-binding domain (or moiety) of the probe, and production of an amplicon involves an interaction between the nucleic acid moieties and/or a further functional moiety which is carried by the other probe(s). Thus amplicon production is dependent on an interaction between the probes (more particularly by the nucleic acid or other functional moieties/domains carried by them) and hence only occurs when both the necessary two (or more) probes have bound to the analyte, thereby lending improved specificity to the detection system.

Proximity-probe based detection assays, and particularly proximity ligation assays permit the sensitive, rapid and convenient detection or quantification of one or more analytes in a sample by converting the presence of such an analyte into a readily detectable or quantifiable nucleic acid-based signal.

Proximity probes of the art are generally used in pairs, and individually consist of an analyte-binding domain with specificity to the target analyte, and a functional domain, e.g. a nucleic acid domain coupled thereto. The analyte-binding domain can be for example a nucleic acid “aptamer” (Fredriksson et al (2002) Nat Biotech 20:473-477) or can be proteinaceous, such as a monoclonal or polyclonal antibody (Gullberg et al (2004) Proc Natl Acad Sci USA 101:8420-8424). The respective analyte-binding domains of each proximity probe pair may have specificity for either the same or different binding sites on the analyte. The analyte in the assay described herein is typically an antibody or fragments of an antibody that is present in a biological sample (e.g., blood) from a subject. In some instances, the subject has an infection (e.g., a viral or bacterial infection) and may have circulating antibodies (e.g., neutralizing antibodies) that are specific to the particular pathogen causing the infection. When a proximity probe pair come into close proximity with each other, which will occur when both are bound to their respective sites on the same analyte molecule (which may be a complex of interacting molecules). i.e. upon coincident binding of the probes to the target analyte, the functional domains (e.g. nucleic acid domains) are able to interact, directly or indirectly. For example, nucleic acid domains of the proximity probes when in proximity may template the ligation of one or more added oligonucleotides to each other (which may be the nucleic acid domain of one or more proximity probes), including an intramolecular ligation to circularize an added linear oligonucleotide. Various such assay formats are described in WO 01/61037. The circular amplicon thereby generated serves to report the presence or absence of analyte in a sample, and can be qualitatively or quantitatively detected, for example by real-time quantitative PCR (q-PCR).

As described above, the use of unique barcoded sequences facilitates tracing the source of each sample from a pool of samples from a single experiment. “Multiplexing” facilitates simultaneous detection of multiple samples combined into a single reaction. Multiplexing with multiple unique barcode sequences allows detection and source identification of several samples in one experiment.

In one aspect, the present disclosure provides a method for identifying at least one infection in a plurality of biological samples. The method comprises obtaining a plurality of biological samples from a plurality of subjects, providing an array that comprises a plurality of capture agents bound to a plurality of uniquely labeled beads. Each uniquely labeled bead comprises a plurality of a unique capture agent. The array further comprises at least one first oligonucleotide sequence that is designed to be bound to at least one bead. In some embodiments, a plurality of first nucleotide sequences bind to a plurality of beads coated with an antigen (e.g., S protein antigen of COVID19) that specifically binds at least one target protein (e.g., antibody from the biological sample specifically binding to the S protein antigen of COVID19 coated on the bead). The array further comprises at least one secondary antibody conjugated with a second oligonucleotide sequence. When the second oligonucleotide sequence is in close proximity to the first oligonucleotide sequence, a uniquely barcoded circular nucleotide template is designed to be amplified to form a circular amplicon. In some embodiments, the first and the second nucleotide sequences comprise unique barcode sequences. In some embodiments, the first and the second nucleotide sequences comprise spacer sequences (e.g., adapter sequences) that allow the addition of two or more unique barcodes to each of the first and second nucleotide sequences.

In some embodiments, the array is a multiplex array comprising one or more plates with at least 96 wells, at least 384 wells, at least 1536 wells, or more wells. In some embodiments, the first and the second nucleotide sequences comprises at least 384 different barcode sequences in the first oligonucleotide sequence, or the second oligonucleotide sequence, or in combination thereof. In particular embodiments, the array comprises at least 384 unique barcode sequences in the circular amplicon. In all of the embodiments described herein, the plurality of beads is uniquely labeled such that each of the uniquely labeled bead comprises a plurality of a unique capture agent. (e.g., S protein antigen of COVID19). In many embodiments of the method described herein, the beads are incubated with at least two proximity probes. The first proximity probe comprises a first oligonucleotide sequence conjugated to a polypeptide that is designed to be bound to the unique capture agent attached to at least one bead. In specific embodiments, the first oligonucleotide sequence is conjugated through direct covalent interacts with the capture agents coated on the bead. In other specific embodiments, the first oligonucleotide sequence is conjugated through indirect covalent interacts with the capture agents coated on the bead such as, mediated by another polypeptide such as a binding domain comprising, for example an antibody, a scFv domain to the antigen on the bead. The second proximity probe comprises a second oligonucleotide sequence conjugated to an antibody that binds specifically (e.g., with a binding affinity of at least about 10⁻⁴M, usually at least about 10⁻⁸ M or higher, e.g., 10⁻¹⁰M or higher) to the target protein (e.g., antibody against S protein of COVID 19). Upon incubation with the sample comprising the target protein, the two proximity probes are brought into close proximity such that they hybridize to the template circular DNA. The circular DNA template is then amplified to produce circular amplicons that are detected by downstream sequencing. In the embodiments of the method described herein, the circular DNA template will be individually barcoded and will also contain proximity ligation sequences for the detectors. Detecting the amplicon indicates that the corresponding sample obtained from a specific subject has the target protein

The proximity probes are nucleic acid tailed or tagged affinity ligands, for example, conjugate molecules that include an affinity ligand (i.e., analyte binding domain) conjugated to a tag or tail nucleic acid (i.e. nucleic acid domain), where the two components are generally (though not necessarily) covalently joined to each other, e.g. directly or through a linking group. In representative embodiments the “tailed” affinity ligand is made up of an affinity ligand covalently joined to a tag nucleic acid, either directly or through a linking group, where the linking group may or may not be cleavable, e.g. enzymatically cleavable (for example, it may include a restriction endonuclease recognized site), photo labile, etc. In certain embodiments, the affinity ligand (i.e. analyte binding) domain, moiety or component of the nucleic acid tailed affinity ligands or proximity probes is a scFV molecule that has a high binding affinity for a target analyte. By high binding affinity is meant a binding affinity of at least about 10⁻⁴M, usually at least about 10⁻⁸ M or higher, e.g., 10⁻¹⁰M or higher. The affinity ligand may be any of a variety of different types of molecules, so long as it exhibits the requisite binding affinity for the target protein when present as tagged affinity ligand. In certain embodiments, the affinity ligand is a ligand that has medium or even low affinity for its target analyte, e.g., less than about 10⁻⁴M.

In many embodiments of the methods described herein, the affinity ligands are binding domains (e.g., antibodies, as well as binding fragments and mimetics thereof.) Where antibodies are the affinity ligand, they may be derived from polyclonal compositions, such that a heterogeneous population of antibodies differing by specificity are each tagged with the same tag nucleic acid, or monoclonal compositions, in which a homogeneous population of identical antibodies that have the same specificity for the target protein are each tagged with the same tag nucleic acid. As such, the affinity ligand may be either a monoclonal and polyclonal antibody. In yet other embodiments, the affinity ligand is an antibody binding fragment or mimetic, where these fragments and mimetics have the requisite binding affinity for the target protein. For example, antibody fragments, such as Fv. F(ab) and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the present disclosure may be readily prepared using any convenient methodology, such as the methodology disclosed in U.S. Pat. Nos. 5,851,829 and 5,965,371; the disclosures of which are herein incorporated by reference.

Importantly, the affinity ligand will be one that includes a domain or moiety that can be covalently attached to the nucleic acid tail without substantially abolishing the binding affinity for the affinity ligand to its target protein.

In many embodiments of the method described herein, a unique barcode sequence is introduced into each of the circular plasmid. This allows for efficient detection after amplification and avoids having to individually label protein samples with barcoded oligos, a cumbersome and a time-consuming process. In other embodiment, a unique barcode sequence is introduced into each of the proximity probes. The barcode sequence is a unique nucleotide sequence that will facilitate source identification (e.g., sample ID, patient ID, well or plate location of the sample in the array). The length of the barcode sequence may be from 3 to 20 nucleotides, such as from 5 to 15 nucleotides in length. In some embodiments, the barcode sequence may be completely random, that is, any one of A, T, G, and C may be at any position of the barcode sequence. In one exemplary embodiment, the unique DNA barcode is assigned by a computer algorithm directing a liquid handling system in a series of two PCR steps. In other embodiments, the barcode sequences are semi-defined or completely defined. In an exemplary embodiment, the subject information is registered into a database and the subjects are given a uniquely-barcoded (physical) sample collection tube. A robot assigns a unique barcode DNA sequence in the chemistry which will allow for unique identification of the sample throughout the process. In this instance, the vial barcode matches the patient, and a unique DNA barcode primer combination is assigned uniquely to the vial's ID. One “well barcode” set of 384 primers (Set A) is assigned to each subject well in a microwell plate (one well per subject, e.g. in a 384 well plate), and then a second set of 384 primers (Set B) amplifies the products of the plate (one “plate” barcode primer per plate). Thus 384×384 unique combinations which amount to totally 147000 unique samples from the patients can be processed. Each of these assignments is tracked and stored for the deconvolution.

In some embodiments, the proximity probes include one or more adapter regions that are complementary to the target template circular DNA. The template circular DNA also has unique barcode information that is retained during amplification and facilitates source identification of the amplicons during the high throughput sequencing steps. In an exemplary embodiment, a unique DNA barcode is assigned by a computer algorithm to each of the template circular DNA in added to each well of the array. 384 unique circular amplicons represent Set A, then they are amplified by algorithmic addition of one of a further 384 forward and reverse from Set B.

In some embodiments of the method described herein, the amplicons are detected by sequencing. Generation of sequence data is typically performed using a high throughput DNA sequencing system, such as a next generation sequencing (NGS) system, which employs massively parallel sequencing of DNA templates. Exemplary NGS sequencing platforms for the generation of nucleic acid sequence data include, but are not limited to, Oxford Nanopore sequencers (e.g.. Nanopore devices comprising MinION Mk1C, Flongle, MinIon, GridIon and/or PromethION), Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms. In some embodiments of the method, the barcoded amplicons were pooled to create a “library,” and were added to a hybridization reaction mixture and incubated for 12 hours at 65° C. Additional sequences (e.g., adapters) required for either the Illumina MiSeq™ (Illumina. San Diego. CA) or Ion Torrent™ Personal Gene Machine (PGM) (Life Technologies, Grand Island, NY) sequencing platforms were added to the 5′ and 3′ adaptors using fusion primers. The DNA library was divided into two halves. One half was amplified with fusion primers that have a portion complementary to the 5′ and 3′ adaptors and add additional sequences for MiSeq sequencing and the other half was amplified with a set of primers that add additional sequences for PGM sequencing.

In an exemplary embodiment, the amplicons are sequenced and the sequencing file contains (a) dual barcoded amplicons for each of the sample containing the target analyte (e.g., COVID 19 specific antibody, SARS specific antibody, influenza specific antibody) from the plurality of subjects, each uniquely tagged and (b) dual barcoded amplicons for a positive control sequence (synthetic or natural) that confirm the PCR reaction ran properly. The assay results are then read by an algorithm that scans the sequence file for the dual barcode combination that uniquely identifies each patient. Upon detecting a joint sequence in the file, for example. (including the adapters, etc.), the algorithm can positively identify the subject and register them as “positive” in the central database. If a patient has only a positive control and no (e.g.. COVID 19 specific antibody, SARS specific antibody, influenza specific antibody) amplicons, they are assigned a “negative” result. In some embodiments, the reporting system that can forward the results to patients, physicians, or clinics, etc.

The methods provided herein are generally directed to robust and flexible methods and systems for determination of consensus sequence of barcoded amplicons from a plurality of sequence data obtained from different patient population and/or from same patient with one or more pathogenic variants. Technologies and methods for biomolecule sequence determination do not always produce sequence data that is perfect. For example, it is often the case that DNA sequencing data does not unambiguously identify every base with 100% accuracy, and this is particularly true when the sequencing data is generated from a single pass, or “read. In certain embodiments, the current methods comprise algorithms for assimilating nucleic acid sequences into a set of final consensus sequences, more accurately than any one-pass sequence analysis system. In specific embodiments, the current methods comprise algorithms that converts the sequence information from PCR amplicons to raw ONT FAST5 sequencing output files which are then converted to raw FASTA/FASTQ files by the high-accuracy ONT GPU-based base caller. The current methods further comprise algorithms that subject the FASTA/FASTQ files to the HMMER3 and CM sequence alignment and annotation engines to yield sequence reads with dual barcodes that pass minimum Leventshein distance score vs reference barcode candidates. These passing reads in the methods described herein are stored in a central database with full target sequence annotation, model fit, bitscore, barcode locations, barcode distance, and other metrics.

In certain embodiments, the method described herein comprises a multiplexed proximity ligation assay, which enable the simultaneous identification of the target analyte from multiple samples. By modifying the barcodes of the oligonucleotide components (e.g., barcodes of the circular amplicons, barcodes of probe oligos) of the assay, this set-up allows for the simultaneous detection of the target analyte from several patient samples. For example, a multiplex array of 384×384 unique combinations can simultaneously asses quantitatively and qualitatively the presence of target analyte (e.g., IgG or IgM immunoglobulins against S protein of COVID 19) in 147000 distinct patient samples. In a related aspect of the disclosure, the serology assay method described herein has particular utility in a multiplex setting, e.g. to detect more than one target analytes that are determinants of pathogenic infection. This method may be used in combinatorial fashion. For example, it may be used to detect at least two target antibodies that are determinants of COVID 19 and Influenza infection, respectively. In such an embodiment, a circular DNA template with unique barcode and/or a pair of proximity probes with unique barcodes may be provided for each of the target antibodies. In a particular embodiment, the circular fragments have an identifying barcode (e.g., patient identifying barcode) and a disease type barcode but the adapter from probe oligos will be different according to the targets.

A detectable circular DNA amplicon with unique barcode may thus be created in a similar fashion from each pair of proximity probes bound to the same target antibody. The “barcodes” are decoded based on the sequencing. The assay results can be read by an algorithm that scans the sequence file for the unique barcodes that uniquely identifies each sample from each patient. Upon detecting a unique amplicon corresponding to each target antibody, the algorithm can positively identify the subject and register them as “positive” in the central database for each infection. If a patient has a positive control and no amplicons corresponding to any target antibody, they are assigned a “negative” result.

### EXAMPLES

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Example 1: Identification and Screening of Amplification Primers Against Distinct Pathogens

In designing primers against distinct pathogens, selection of primers will be made against genomic regions which are distinct and unique to each pathogen. The resulting amplicons produced by the amplification using the primers selected above, carry the genomic sequence for each of those distinct pathogens. The HMM models will be defined for each of the pathogen sequences and their barcodes, and upon alignment, the models most closely matching (e.g. the alignments with the highest bitscore) the pathogen sequences indicate which pathogen(s) were present in the original sample. These primers can be barcoded (e.g., single stage or dual stage barcoding) as described herein. Barcode sequences and primers are selected from a very large, validated IDT barcode library that has been screened for secondary structure interactions, resulting in a highly optimized, error tolerant barcode design. Non-limiting exemplary barcode sequences are provided in Table 1. The 96 barcode sequences provided in Table 1 (selected from within the 3000+ total barcodes) are maximally Levenshtein-distance separated. The methods described herein can use 384 maximally Levenshtein-distance separated barcode sequences. The selection of barcode sequences is done algorithmically and yields different results depending on the selection size. In may embodiments of the methods and compositions provided herein, the number of barcode sequences selected is based on the size of the barcode pool that the primers are assembled from. Upon sequencing, these barcodes can also be identified and used to assign a patient identity to each sequenced amplicon.

### Example 2: Amplicon Design

Amplicon design begins with pathogen-specific forward and reverse primers that have been synthesized with barcoded sequences and spacer (adapter) sequences on each of the primers' 5′ ends. Upon amplification in the presence of the pathogen's genome, this yields an amplicon pool where each strand of DNA contains the spacers (adapters) and the barcodes. The spacers are essential for the HMM/CM alignment engine to correctly identify barcodes, and to be able to resolve distinct barcodes in the final sequence. Non-limiting example of the adapter sequence is provided herein in the polynucleotide sequence set forth in ACACTGACGACATGGTTCTACA (SEQ ID NO.:21) and TACGGTAGCAGAGACTTGGTCT (SEQ ID NO.:22).

### Example 3: Analysis of Sequence Variants

Pathogenic variants can be readily identified if the primers used to form the amplicons span a genomic region (e.g. the receptor binding domain of the SARS-CoV-2 spike protein) which is known to carry hallmark mutations specific to each variant. Upon sequencing a pathogen sample, the template sequence (e.g. non-barcode, non-spacer) can be aligned in the best-scoring amplicons to reference genomic databases. Then, by sequence similarity or identity, a determination of a close match of a previously-described sequence to the template sequence, can be made. Alternatively, a multiple sequence alignment of template sequences from each patient can be performed to generate a consensus sequence. Hundreds to tens of thousands of amplicon sequences per patient are frequently obtained, allowing for a very robust consensus sequence even in the presence of the occasional sequencer error. The consensus sequence can then be aligned to sequences in a genomic or protein reference database (e.g. Genbank or a custom-made reference genome database).

### Example 4: SARS-CoV-2 Viral Detection

### A. Primer Design and Generation of the Barcoded Amplicon

PCR primers and ligation reactions designed to maximize throughput while generating highly computationally-optimized amplicons (motifs, barcodes, spacers, and well-defined viral inserts).

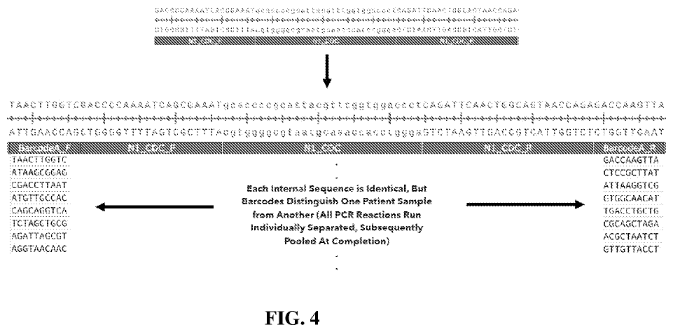

Biological samples (e.g., blood, saliva or mucus) from patients with known or suspected SARS-CoV-2 exposure was obtained. The SARS-Cov-2 genome was selected as an exemplary target genome. Unique sequence segments of about 7 to 12 nucleobases in length corresponding to the to the nucleotides encoding the E-Guelph, N_HKU, N2, Orflab proteins, were identified. Frequency of occurrence and selectivity ratio values were determined. In most cases, the primers were designed to hybridize with 100% complementarity to its corresponding genome sequence segment (e.g., segments corresponding to the nucleotides encoding the E-Guelph, N_HKU, N2, Orflab proteins). In a few other cases, degenerate primers were prepared. The degenerate bases of the primers occur at positions complementary to positions having ambiguity within the target. Standard qPCR Primers amplify a small segment of DNA for probe hybridization. As shown in FIG. 4, the qPCR amplicons are identical from patient to patient.

FIG. 4 shows an exemplary amplicon generated by the amplification of the target N1 protein in the SARS-Cov2 genome using primers about 20 nucleobases in length ligated to a barcoded sequence that is unique for each patient sample. As shown in FIG. 4, while each target sequence is identical, the unique barcodes at the ends of the sequences distinguish individual patient samples from one another, allowing for sample pooling while retaining sample ID.

### B. Multiplex PCR and Sequencing

FIG. 5A shows sequence labeling and scoring data of an exemplary target E-Guelph protein from the SARS-Cov2 genome. The PCR amplicons from pooled library preparations were sequenced on ONT MinION or GridION to obtain raw ONT FAST5 sequencing output files. The output files were subjected to high-accuracy ONT GPU-based base caller to yield raw FASTA/FASTQ files. The FASTA/FASTQ files were run on the HMMER3 and CM sequence alignment and annotation engines. The HMM/CM engines apply the statistical pattern classification algorithm to generate the consensus sequence by a) maximizing a likelihood based upon the replicate sequence reads, and/or b) using a context dependent alignment model parameter based upon a whole genome multiple sequence alignment. FIG. 5A shows the bit score and the alignments of the barcode and viral insert regions.

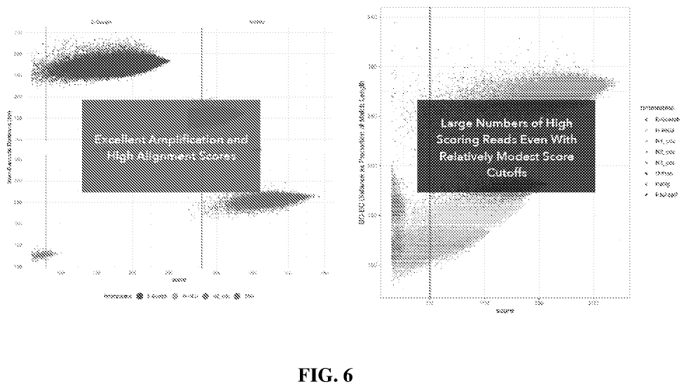

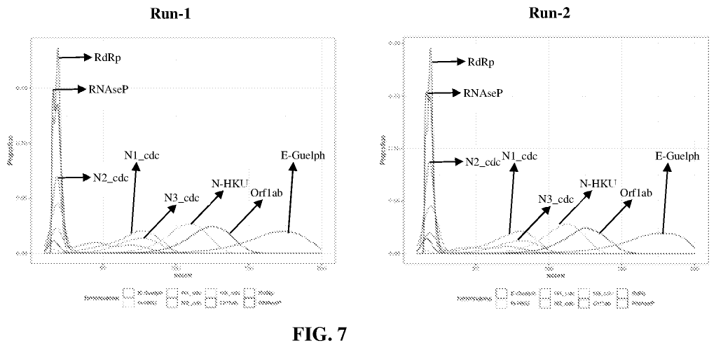

A wide range of both SARS-CoV-2 gene targets (e.g., E-Guelph, N-HKU) and controls (e.g., TME) for use in the SARSCoV-2 viral detection assay were evaluated. Multiple PCR master mixes were evaluated. All targets displayed adequate PCR amplification, with superiority in longer genes and in NEB master mixes (Luna-Taq selected). FIG. 6 shows the multiplexed PCR and sequencing results from the SARS-CoV-2 gene targets. The results demonstrate excellent amplification and high alignment scores. Large numbers of high scoring reads were obtained even with relatively modest score cutoffs. As shown in FIG. 7 and Table 3, high reproducibility with nearly identical-cross-run sequence recovery was obtained across the multiple sequencing runs.

TABLE 3

| | | | | Read | Read | Read |
|---|---|---|---|---|---|---|
| | | | | Count | Count | Count |
| | Target | | Viral | HMM, | HMM, | CM, |
| Sample | Gene | Barcode | Copies | 20200806 | 20200810 | 20200806 |
| 1 | N1_cdc | IDT10_i7_1 | 10 | 4242 | 3199 | 13038 |
| 2 | N1_cdc | IDT10_i7_2 | 100 | 803 | 509 | 2988 |
| 3 | N1_cdc | IDT10_i7_3 | 1000 | 1353 | 1085 | 5004 |
| 4 | N1_cdc | IDT10_i7_4 | 10000 | 5231 | 3445 | 15358 |
| 5 | N2_cdc | IDT10_i7_5 | 10 | 223 | 135 | 742 |
| 6 | N2_cdc | IDT10_i7_6 | 100 | 72 | 39 | 244 |
| 7 | N2_cdc | IDT10_i7_7 | 1000 | 506 | 260 | 6888 |
| 8 | N2_cdc | IDT10_i7_8 | 10000 | 4374 | 3030 | 17206 |
| 9 | N3_cdc | IDT10_i7_9 | 10 | 38 | 20 | 214 |
| 10 | N3_cdc | IDT10_i7_10 | 100 | 529 | 378 | 1780 |
| 11 | N3_cdc | IDT10_i7_11 | 1000 | 23 | 16 | 192 |
| 12 | N3_cdc | IDT10_i7_12 | 10000 | 5803 | 3810 | 19474 |
| 21 | Orf1ab | IDT10_i7_21 | 10 | 1 | 1 | 2 |
| 22 | Orf1ab | IDT10_i7_22 | 100 | 10 | 5 | 28 |
| 23 | Orf1ab | IDT10_i7_23 | 1000 | 2914 | 2112 | 7504 |
| 24 | Orf1ab | IDT10_i7_24 | 10000 | 1528 | 1379 | 6078 |
| 25 | E-Guelph | IDT10_i7_25 | 10 | 7 | 3 | 328 |
| 26 | E-Guelph | IDT10_i7_26 | 100 | 597 | 321 | 3564 |
| 27 | E-Guelph | IDT10_i7_27 | 1000 | 1344 | 784 | 23594 |
| 28 | E-Guelph | IDT10_i7_28 | 10000 | 10144 | 5814 | 237034 |
| 29 | N-HKU | IDT10_i7_29 | 10 | 234 | 204 | 1688 |
| 30 | N-HKU | IDT10_i7_30 | 100 | 418 | 332 | 1852 |
| 31 | N-HKU | IDT10_i7_31 | 1000 | 6285 | 5899 | 20032 |
| 32 | N-HKU | IDT10_i7_32 | 10000 | 15885 | 15870 | 61674 |

### Example 4: Point of Care Data from Patients Exposed to SARS-CoV2

Biological samples (e.g., blood, saliva and/or mucus) were obtained from 7 patients with known or suspected SARS-CoV2 exposure. Target specific primers specific were designed to hybridize with 100% complementarity to the nucleotides encoding the E-Guelph, N_HKU, N2, Orflab proteins. PCR amplicons were generated by the amplification of the target proteins E-Guelph, N_HKU, N2, Orflab proteins in the SARS-Cov2 genome using primers about 20 nucleobases in length ligated to a barcoded sequence that is unique for each patient sample, qPCR indicated that 3 out of 7 patients were negative. High quality reads for all the qPCR negative samples were obtained using the massively parallel diagnostic method described herein. The results are summarized in Table 4.

TABLE 4

| Target | Barcode | Read Count | Subject | qPCR | Notes |
|---|---|---|---|---|---|
| E-Guelph | 27 | 138 | A | + | Mild symptoms ~1 day prior |
| N-HKU | 31 | 590 | A | + | Mild symptoms ~1 day prior |
| N2_cdc | 7 | 855 | B | − | Mild symptoms ~4 days prior |
| N2_cdc | 8 | 26 | C | + | Unknown symptoms (exposed) |
| E-Guelph | 25 | 6,189 | D | + | Mild symptoms ~2 days after |
| E-Guelph | 28 | 606,017 | D | + | Mild symptoms ~2 days after |
| N-HKU | 29 | 81,562 | D | + | Mild symptoms ~2 days after |
| N-HKU | 32 | 204,736 | D | + | Mild symptoms ~2 days after |
| N2_cdc | 6 | 257 | E | − | Mild symptoms ~1 day prior |
| Orf1ab | 22 | 8 | E | − | Mild symptoms ~1 day prior |
| E-Guelph | 26 | 161 | F | + | Mild symptoms ~1 day prior |
| N-HKU | 30 | 280 | F | + | Mild symptoms ~1 day prior |
| N2_cdc | 5 | 19 | G | − | Asymptomatic (exposed) |
| TM3 | 1 | 21,955 | MS2 | n/a | MS2 Phage control RNA |

### Other Embodiments

All publications, patents, and patent applications mentioned in this specification are incorporated herein by reference to the same extent as if each independent publication or patent application was specifically and individually indicated to be incorporated by reference.

While the present disclosure has been described in connection with specific embodiments thereof, it will be understood that it is capable of further modifications and this disclosure is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims.

Other embodiments are within the claims.

1. A method for identifying at least one target nucleic acid, the method comprising:
a) obtaining a plurality of biological samples from a plurality of subjects;
b) obtaining total nucleic acid from each of the biological samples, wherein the total nucleic acid comprises a plurality of polynucleotides;
wherein, if the plurality of polynucleotides comprise RNA molecules, step b) further comprises obtaining cDNA reverse-transcribed from the RNA or reverse-transcribing cDNA from the RNA before performing the amplification in step c),
c) subjecting the plurality of polynucleotides to amplification using an amplification mixture to produce a plurality of amplicons, wherein the amplification mixture comprises a plurality of primers, a first unique barcode sequence and its reverse complement, and at least one pair of adapter sequences, wherein each of the plurality of the primers comprise a set of nucleotides that are complementary to each of the polynucleotides that they bind to, wherein the first unique barcode sequence identifies the biological sample obtained from the specific subject, wherein the pair of adapter sequences flank the first unique barcode sequence and its reverse complement, and wherein each of the plurality of amplicons comprise polynucleotides from a target amplified region or a control region;
d) detecting each of the plurality of amplicons; and
e) determining a category of the plurality of amplicons;
wherein the determining the category of each the plurality of amplicons comprising the polynucleotides from the target amplified region indicates that the corresponding subject has the target nucleic acid.

2. The method of claim 1, wherein the plurality of polynucleotides in step b) comprises RNA molecules, and wherein a reverse transcriptase is added in step b) to obtain a plurality of cDNAs that will be subjected to amplification in step c).

3. The method of claim 2, wherein the plurality of polynucleotides in step b) further comprises DNA molecules.

4. The method of claim 1, wherein the target nucleic acid is obtained from a sample comprising one or more pathogens selected from the group consisting of a RNA virus, a DNA virus, a fungus, a parasite and a bacterium.

5. The method of claim 4, wherein the pathogen is selected from a group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemonhagic Fever virus Chapare, South American Haemorrhagic Fever virus Guanarito, South American Haemorrhagic Fever virus Junin, South American Haemorrhagic Fever virus Machupo, South American Haemonhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above.

6. The method of claim 1, wherein the pair of adapter sequences separate the first unique barcode sequence and its reverse complement from a second unique barcode sequence and its reverse complement.

7. The method of any one of claims 1-6, wherein the sample is selected from the group consisting of blood, mucus, saliva, sweat, tears, fluids accumulating in a bodily cavity, urine, ejaculate, vaginal secretion, cerebrospinal fluid, lymph, feces, sputum, decomposition fluid, vomit, sweat, breast milk, serum, and plasma.

8. The method of claim 6, wherein the RNA virus is SARS-CoV-2.

9. The method of claim 7, wherein the sample is saliva.

10. The method of claim 1, wherein detecting comprises sequencing the plurality of amplicons comprising the pair of adapter sequences and the first unique barcode sequence and its reverse complement.

11. The method of claim 6, wherein detecting comprises sequencing the plurality of amplicons comprising the pair of adapter sequences, the first unique barcode sequence and its reverse complement, and the second unique barcode sequence and its reverse complement.

12. The method of claim 10 or 11, wherein the detecting is performed by reading a sequencing data file with a suite of programs.

13. The method of claim 12, wherein the sequencing data file is a FASTA/FASTQ formatted file.

14. The method of claim 12, wherein the suite of programs comprise HMMER/Infernal alignment engines.

15. The method of any one of claims 10-14, further comprising sequencing at least one positive control sample, wherein the positive control sample comprises the target nucleic acid.

16. The method of any one of claims 10-14, further comprising sequencing at least one positive control sample, wherein the positive control sample is a Bacteriophage MS2.

17. The method of any one of claims 10-14, further comprising sequencing at least one positive control sample, wherein the positive control sample is a MS2 template nucleic acid.

18. The method of any one of claims 10-14, further comprising sequencing at least one positive control sample, wherein the positive control sample is a RNAseP or another non-pathogen gene.

19. The method of any one of claims 10-14, further comprising sequencing at least one positive control sample, wherein the positive control sample is a nucleic acid from a human housekeeping gene GAPDH or beta-actin.

20. The method of any one of claims 1 to 17, wherein the plurality of primers comprises at least 96 different barcoded primers.

21. The method of any one of claims 1 to 17, wherein the method comprises identifying two or more target nucleic acids.

22. The method of claim 21, wherein the two or more target nucleic acids are pathogenic determinants, or encode for pathogenic determinants, of a single pathogen.

23. The method of claim 22, wherein the two or more target nucleic acids are pathogenic determinants, or encode for pathogenic determinants, of a virus.

24. The method of claim 23, wherein the virus is SARS-CoV-2.

25. The method of claim 24, wherein the pathogenic determinants are selected from the group consisting of a spike protein (S), a receptor-binding domain (RBD), a S1 protein, a S2 protein, E gene, S gene, Orflab gene, N-terminal Spike protein domain, a whole protein (S1+S2), and a nucleocapsid (N) protein.

26. The method of claim 22, wherein the two or more target nucleic acids are pathogenic determinants, or encode for pathogenic determinants, of at least two different pathogens selected from a group consisting of a RNA virus, a DNA virus, a fungus, a parasite and a bacterium.

27. The method of claim 26, wherein the two different RNA viruses are SARS-CoV-2 and Influenza.

28. The method of any one of claims 1-27, wherein the amplification is a rolling circle amplification.

29. The method of any one of claims 1-27 wherein the amplification is a polymerase chain reaction amplification.

30. A multiplex of array for detecting at least one target protein from multiple samples, the array comprising:
a. a plurality of capture agents bound to a plurality of uniquely labeled beads, wherein each unique labeled bead comprises a plurality of a unique capture agent;
b. at least one first oligonucleotide sequence that is designed to be bound to at least one bead;
wherein the bead is coated with an antigen that specifically binds at least one target protein;
c. at least one secondary antibody conjugated with a second oligonucleotide sequence which is designed to be amplified to form a circular amplicon when the second oligonucleotide sequence is in close proximity to the first oligonucleotide sequence; and
d. at least one unique nucleotide barcode sequence in the circular amplicon.

31. The multiplex of array of claim 30, wherein the first oligonucleotide sequence, or the second oligonucleotide sequence, or both, comprise at least one unique barcode sequence.

32. The multiplex of array of claim 31, wherein array comprises at least 384 different barcode sequences in the first oligonucleotide sequence, or the second oligonucleotide sequence, or in combination thereof.

33. The multiplex of array of claim 30, wherein array comprises at least 96 different barcode sequences in the circular amplicon.

34. The multiplex of array of claim 30, wherein the first oligonucleotide sequence is covalently bound to a polypeptide coated on the bead.

35. The multiplex of array of claim 30, wherein the first oligonucleotide sequence is covalently bound to an antibody or an antibody fragment, wherein the antibody or the antibody fragment bind to a polypeptide coated on the bead.

36. A method for identifying at least one infection in a plurality of biological samples, the method comprising:
a. providing the multiplex array of claim 30;
b. incubating a plurality of biological samples with a plurality of beads under conditions sufficient for at least one target protein to bind to the unique capture agent of at least one of the beads;
c. washing the beads to remove any proteins that do not bind to the unique capture agents;
d. incubating the beads with a plurality of secondary antibodies under conditions wherein each of the plurality of the secondary antibodies forms a complex with at least one target protein, and wherein a plurality of complexes corresponding to the number of the secondary antibodies bound to the plurality of target proteins, are formed
e. washing the beads to remove any secondary antibodies that do not form the complex;
f. incubating the plurality of complexes under conditions to allow hybridization of each of the second oligonucleotide sequence to each of the first oligonucleotide sequence such that they form a circular amplicon, wherein a plurality of amplicons are generated corresponding to the number of the plurality of complexes, and wherein each of the plurality of amplicons comprise polynucleotides from a target amplified region or a control region, a unique barcode sequence and its reverse complement, and a first pair of adapter sequences;
g. subjecting the plurality of amplicons to amplification;
h. pooling the beads in the array and simultaneously detecting the plurality of amplicons by high throughput sequencing; and
i. determining a category of the plurality of amplicons;
wherein determining the category of each the plurality of amplicons comprising the polynucleotides from the target amplified region indicates infection in the corresponding biological sample.

37. The method of claim 36, wherein each of the sample from the plurality of samples is uniquely barcoded prior to the incubating step b.

38. The method of claim 37, wherein the multiplex array comprises at least 96 different barcode sequences in the first oligonucleotide sequence, or the second oligonucleotide sequence, or in combination thereof.

39. The method of claim 37, wherein array comprises at least 96 different barcode sequences in the plurality of the circular amplicons.

40. The method of claim 36, wherein the target protein is an antibody or an antibody fragment.

41. The method of claim 40, wherein the antibody is an IgM antibody.

42. The method of claim 40, wherein the antibody is an IgG antibody.

43. The method of claim 40, wherein the antibody or the antibody fragment binds specifically to an antigen from a group consisting of a bacterium, a RNA virus and a DNA virus.

44. The method of claim 43, wherein the antibody or the antibody fragment binds specifically to an antigen from a group consisting pathogen is selected from a group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above.

45. The method of claim 44, wherein the antibody or the antibody fragment binds specifically to an antigen from SAR-CoV-2.

46. The method of claim 44, wherein the antibody or the antibody fragment binds specifically to an antigen selected from the group consisting of a S protein, RBD of S protein, a S1 protein, a S2 protein, E gene, S gene, Orflab gene, N-terminal Spike protein domain, a whole protein (S1+S2), and a N protein.

47. The method of any one of claims 36-47, wherein the sample is selected from the group consisting of blood, mucus, saliva, sweat, tears, fluids accumulating in a bodily cavity, urine, ejaculate, vaginal secretion, cerebrospinal fluid, lymph, feces, sputum, decomposition fluid, vomit, sweat, breast milk, serum, and plasma.

48. The method of claim 47, wherein the sample is blood.

49. The method of claim 47, wherein the sample is saliva.

50. A method for detecting sequence variants in a nucleic acid sample, the method comprising the steps of:
a. performing an amplification reaction with a amplification mixture to produce a plurality of amplicons, wherein the amplification mixture comprises the nucleic acid sample, a plurality of primers, a first unique barcode sequence and its reverse complement, and a first pair of adapter sequences, wherein each of the plurality of the primers comprise a set of nucleotides that are complementary to each of the polynucleotides that they bind to, wherein the first unique barcode sequence and its reverse complement identify the sample obtained from a specific subject, wherein the pair of adapter sequences flanks the first unique barcode sequence and its reverse complement, and wherein each of the plurality of amplicons comprise polynucleotides from a target amplified region or a control region;
wherein, if the sample of nucleic acid comprises RNA molecules, step a) further comprises obtaining cDNA reverse-transcribed from the RNA or reverse-transcribing cDNA from the RNA before performing the amplification reaction,
b. detecting, and optionally quantitating, the plurality of amplicons;
c. determining a category of the plurality of amplicons; and
d. detecting one or more sequence variants in the plurality of amplicons from step c.

51. The method of claim 50, wherein detecting in step b comprises sequencing each of the plurality of amplicons comprising the first pair of adapter sequences and the first unique barcode sequence and its reverse complement.

52. The method of claim 50, wherein the first pair of adapter sequences separate the first unique barcode sequence and its reverse complement from a second unique barcode sequence and its reverse complement.

53. The method of claim 52, wherein detecting in step b comprises sequencing each of the plurality of amplicons comprising the first pair of adapter sequences, the first unique barcode sequence and its reverse complement, the second unique barcode sequence and its reverse complement.

54. The method of claim 52, wherein detecting in step b further comprises sequencing a second pair of adapter sequences.

55. The method of any one of claims 50-54, wherein the detecting in step b is performed by reading a sequencing data file with a suite of programs.

56. The method of claim 55, wherein the sequencing data file is in a FASTA/FASTQ format.

57. The method of claim 55, wherein the suite of programs comprise HMMER/Infernal alignment engines.

58. The method of any one of claims 50-54, wherein the detecting in step d comprises performing a multiple sequence alignment with one or more reference sequences.

59. The method of claim 58, wherein the sequence alignment is performed by a HMM profile Hidden Markov Model (HMM) engine, a covariance model (CM) engine or a combination thereof.

60. The method of claim 50 further comprising correlating the sequence variants with a diagnosis or a prognosis of an infection.

61. The method of claim 60, wherein the infection is caused by one or more pathogens selected from the group consisting of a RNA virus, a DNA virus, a fungus, a parasite and a bacterium.

62. The method of claim 61, wherein the pathogen is selected from a group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papilloma virus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemonhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucci, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above.

63. The method of claim 62, wherein the pathogen is SAR-CoV-2.

64. The method of claim 63, wherein the sequence variants are in a region encoding an antigen selected from the group consisting of a S protein, RBD of S protein, a S1 protein, a S2 protein, E gene, S gene, Orflab gene, N-terminal Spike protein domain, a whole protein (S1+S2), and a N protein.

65. The method of claim 64, wherein the sequence variants comprise mutations selected from a group consisting of T95I, D253G, L452R, E484K, S477N, N501Y D614G and A701V.

66. The method of any one of claims 1, 36 or 50, wherein the detecting the plurality of amplicons comprises:
a. obtaining a pooled sequence dataset of the plurality of amplicons, wherein each unique barcode sequence and its reverse complement on each amplicon is unique to a single sample, wherein the unique barcode sequence and its reverse complement of each amplicon from a first single sample is distinct from the unique barcode sequences and their reverse complements of the other amplicons in the plurality of amplicons;
b. performing base calling;
c. aligning the sequence data of the plurality of amplicons to a pre-defined, annotated HMM or CM gene model;
d. assigning a rank to each of the HMM/CM alignments, wherein the rank is a probability score or a bit score;
e. filtering the sequence data to obtain a positionally annotated sequence alignments, denoting the barcode(s) within each amplicon as well as the location of the barcode and the adapter within the amplicon's sequence; and
f. performing at least steps b, c, d, and e using a suitably programmed computer.

67. The method of claim 66, wherein the base calling is performed with a high-accuracy ONT GPU-based base caller.

68. The method of claim 66, wherein the base calling yields raw FASTA/FASTQ files.

69. The method of claim 66, wherein the aligning is performed by a profile HMM engine, a CM engine or a combination thereof.

70. The method of claim 69, wherein the HMM engine, the CM engine or the combination thereof, assigns a per-nucleotide annotation for one or more sequence feature selected from a group consisting of the barcode, the target amplified region, the primer, and the adapter.

71. The method of claim 69, wherein the HMM engine comprises a HMMER software program that yields a plurality of sequence alignments.

72. The method of claim 71, wherein the plurality of sequence alignments comprise annotations for the first unique barcode sequence and its reverse complement.

73. The method of claim 72, wherein the filtering comprises assigning a pass score or a fail score to the sequence alignments with the first unique barcode sequence and its reverse complement, wherein the plurality of sequence alignments with the first unique barcode sequence and its reverse complement are assigned a passing score if they pass a minimum Levenshtein distance score relative to a set of reference barcoded sequences and if they pass a minimum bitscore threshold for alignments.

74. The method of claim 71, wherein the plurality of sequence alignments comprise annotations for a dual barcode on a per-nucleotide basis, wherein the dual barcode comprises a first unique barcode sequence and its reverse complement and a second unique barcode sequence and its reverse complement.

75. The method of claim 71, wherein the HMMER software program yields sequence alignments with annotations for the first pair of adapter sequences.

76. The method of claim 74, wherein the filtering comprises assigning a pass score or a fail score to the sequence alignments with dual barcodes, wherein the plurality of sequence alignments with dual barcodes are assigned a passing score if they pass a minimum Levenshtein distance score relative to a set of reference barcoded sequences and if they pass a minimum bitscore threshold for alignments.

77. The method of any one of claims 73 or 76, wherein the sequence alignments with the passing score are stored in a central database.

78. The method of claim 77, wherein the sequence alignments with the passing score correspond to a direct quantitative representation of a pathogen load in the sample.

79. The method of claim 77, wherein the database comprises:
information of a unique barcode assigned to a sample collection tube;
information of a set of at least 96 unique well barcodes, wherein each unique barcode is assigned to each sample;
information of a set of at least 96 unique plate barcodes, wherein each unique barcode is assigned to a unique plate;
information of a set of sequence data, wherein the sequence data comprises sequencing data from the plurality of amplicons; and
a report, wherein the report comprises source identifying information of each sample and information on whether the sample is positive or negative for the presence of the target protein.

80. The method of claim 79 further comprising providing the report to corresponding subjects, or to a clinic or to a physician, wherein the sample is obtained from a subject.

81. A composition comprising an amplicon, wherein the amplicon comprises a first unique barcode sequence and its reverse complement, a pair of target-specific primers, a target amplified region and a first pair of adapter sequences, wherein the pair of target specific primers is made up of a forward primer and a reverse primer, each having sequences complementary to the priming sites in the target amplified region, wherein each of the forward primer and the reverse primer flanks the target amplified region, wherein the target specific primers are flanked by the first unique barcode sequence and its reverse complement, and wherein the first unique barcode sequence and its reverse complement are flanked by the first pair of adapter sequences.

82. The composition of claim 81, further comprising a second unique barcode sequence and its reverse complement and a second pair of adapter sequences, wherein the second unique barcode sequence and its reverse complement and the second pair of adapter sequences, are ligated to the amplicon.

83. The composition of claim 82, wherein first pair of adapter sequences are flanked by the second pair of adapter sequences, and wherein the second pair of adapter sequences are flanked by the second unique barcode sequence and its reverse complement.

84. The composition of any one of claims 81-83, wherein the target amplified region is amplified from a genomic region of a pathogen encoding for a gene or protein, and wherein the pathogen is selected from the group consisting of Acinetobacter baumannii, Adenovirus, African horse sickness virus, African swine fever virus, Anclostoma duodenale, Ascaris lumbricoides, Aspergillus flavus, Aspergillus fumigatus, Aspergillus niger, Aspergillus oryzae, Avian influenza virus, Bacillus anthracis, Bacillus anthracis Pasteur strain, Bacillus cereus Biovar anthracis, Brucella abortus, Brucella melitensis, Brucella suis, Burkholderia mallei, Burkholderia pseudomallei, Candida albicans, Candida dubliniensis, Candida glabrata, Candida krusei, Candida tropicalis, Chlamydia pneumoneae, Chlamydia trachomatous, Classical swine fever virus, Clostridium difficile, Coccidioides immitis, Coccidioides posadasii, CoV-229E, CoV-HKU1, CoV-NL63, CoV-OC43, Coxasckie virus A, Coxasckie virus B, Coxiella burnetii, Crimean-Congo haemorrhagic fever virus, Cytomegalovirus, Dengue virus, Dracunculus medinensis, Eastern Equine Encephalitis virus, Ebola virus, Echinococcus granulosus, Echinococcus multilocularis, Enterobacter cloacae, Enterococcus faecium, Enteroviruses, Epstein-Barr virus, Escherichia coli, Fasciola giganta, Fasciola hepatica, Foot-and-mouth disease virus, Francisella tularensis, Goat pox virus, Haemophilus influenza, Helicobacter pylori, Hendra virus, Hepatitis A virus, Hepatitis B virus, Hepatitis C virus, Histoplasma capsulatum, Histoplasma duboisii, Human herpesviruses HHV6, Human herpesviruses HHV7, Human herpesviruses HHV8, Human herpesviruses HSV1, Human herpesviruses HSV2, Human immunodeficiency virus, Human papillomavirus, Influenza virus A, Influenza virus B, Klebsiella pneumonia, Kyasanur Forest disease virus, Lassa virus, Legionella pneumophila, Leishmania promastigotes, Lujo virus, Lumpy skin disease virus, Marburg virus, Measles virus, methicylin resistant Staphylococcus aureus, Monkeypox virus, Mumps virus, Mycobacterium abscessus, Mycobacterium avium, Mycobacterium bovis, Mycobacterium canettii, Mycobacterium leprae, Mycobacterium tuberculosis, Mycobacterium ulcerans, Mycoplasma capricolum, Mycoplasma mycoides, Mycoplasma pneumoneae, Necator americanus, Neisseria gonorrhoeae, Newcastle disease virus, Nipah virus, Nocardia beijingensis, Nocardia cyriacigeorgica, Nocardia farcinica, Norovirus GI, Norovirus GII, Norwalk virus, Omsk hemorrhagic fever virus, Onchocerca volvulus, oncogenic Human papillomavirus, Parainfluenza virus, Parasites, Penicilliosis marneffei, Peste des petits ruminants virus, Pneumocystis jirovecii, Polyomavirus, Proteus mirabilis, Pseudomonas aeruginosa, Rabies virus, Reconstructed replication competent forms of the 1918 pandemic influenza virus containing any portion of the coding regions of all eight gene segments, respiratory syncytial virus, Rhinoviruses, Rickettsia prowazekii, Rift Valley fever virus, Rinderpest virus, Rotavirus A, Rotavirus B, Rotavirus C, Rotavirus G2, Rubella virus, SARS-associated coronavirus (SARS-CoV), SARS-CoV-1, SARS-CoV-2, Schistosoma haematobium, Schistosoma japonicum, Schistosoma mansoni, Sheep pox virus, South American Haemorrhagic Fever virus Chapare, South American Haemorrhagic Fever virus Guanarito, South American Haemorrhagic Fever virus Junin, South American Haemorrhagic Fever virus Machupo, South American Haemorrhagic Fever virus Sabia, Staphylococcus aureus, Staphylococcus saprophyticus, Streptococcus pneumoneae, Swine vesicular disease virus, Taenia solium, Tick-borne encephalitis complex (flavi) virus Far Eastern subtype, Tick-borne encephalitis complex (flavi) virus Siberian subtype, Tobacco mosaic virus, Torque teno virus, Trichuris trichiura, Trypanosoma brucei, Trypanosoma cruzi, Variola major virus (Smallpox virus), Variola minor virus (Alastrim), Venezuelan equine encephalitis virus, Wuchereria bancrofti, Yersinia pestis and a pathogen sharing a distinctive nucleic acid sequences any one of the pathogen described above.

85. The composition of claim 84, wherein the pathogen is SARS-CoV-2.

86. The composition of claim 85, wherein the target amplified region is amplified from a genomic region encoding for protein selected from the group consisting of a S protein, RBD of S protein, a S1 protein, a S2 protein, E gene, S gene, Orf1ab gene, N-terminal Spike protein domain, a whole protein (SI-+S2), and a N protein.

87. The composition of claim 86, wherein the target amplified region is amplified from a region encoding the S protein.

88. The composition of claim 86, wherein the target amplified region is amplified from a region encoding the RBD of the S protein.

89. The composition of claim 86, wherein the target amplified region is amplified from a region encoding the N protein.

90. The composition of any one of claims 81-89, wherein the unique barcode sequences and their reverse complements have a maximal Levenshtein distance from all other barcodes.

91. The composition of claim 90, wherein the unique barcode sequences comprise any one of the polynucleotide sequences set forth in SEQ ID NOs.:23-118.

92. The composition of any one of claims 85-89, wherein the pair of target-specific primers is selected from a group of forward and reverse primers consisting of
Forward Primer:
(SEQ ID NO.: 3)
GACCCCAAAATCAGCGAAAT
and
Reverse Primer:
(SEQ ID NO.: 4)
TCTGGTTACTGCCAGTTGAATCTG;
Forward Primer:
(SEQ ID NO.: 5)
TTACAAACATTGGCCGCAAA
and
Reverse Primer:
(SEQ ID NO.: 6)
GCGCGACATTCCGAAGAA;
Forward Primer:
(SEQ ID NO.: 7)
GGGAGCCTTGAATACACCAAAA
and
Reverse Primer:
(SEQ ID NO.: 8)
TGTAGCACGATTGCAGCATTG;
Forward Primer:
(SEQ ID NO.: 9)
GTGARATGGTCATGTGTGGCGG
and
Reverse Primer:
(SEQ ID NO.: 10)
CARATGTTAAASACACTATTAGCATA;
Forward Primer:
(SEQ ID NO.: 11)
ACAGGTACGTTAATAGTTAATAGCGT
and
Reverse Primer:
(SEQ ID NO.: 12)
ATATTGCAGCAGTACGCACACA;
Forward Primer:
(SEQ ID NO.: 13)
CCCTGTGGGTTTTACACTTAA
and
Reverse Primer:
(SEQ ID NO.: 14)
ACGATTGTGCATCAGCTGA;
Forward Primer:
(SEQ ID NO.: 15)
GTACTCATTCGTTTCGGAAGAG
and
Reverse Primer:
(SEQ ID NO.: 16)
CCAGAAGATCAGGAACTCTAGA;

93. The composition of any one of claims 81-83, wherein the first pair of adapter sequences and the second pairs of adapter sequences are identical comprise between 10 to15 nucleotides.

94. The composition of claim 93, wherein the pair of adapter sequences comprise 10 nucleotides.

95. The composition of claim 94, wherein the pair of adapter sequences comprise polynucleotide sequence as set forth in ACACTGACGACATGGTTCTACA (SEQ ID NO.:21) and TACGGTAGCAGAGACTTGGTCT (SEQ ID NO.:22).


SEQUENCE LISTING (207 sequences)

SEQ ID NO: 1 - DNA, 30 bp, Artificial Sequence
  Description of Artificial Sequence Synthetic primer
taacttggtcgaccccaaaatcagcgaaat

SEQ ID NO: 2 - DNA, 34 bp, Artificial Sequence
  Description of Artificial Sequence Synthetic primer
gtctaagttgaccgtcattggtctattgaaccag

SEQ ID NO: 3 - DNA, 20 bp, Artificial Sequence
  Description of Artificial Sequence Synthetic primer
gaccccaaaatcagcgaaat

SEQ ID NO: 4 - DNA, 24 bp, Artificial Sequence
  Description of Artificial Sequence Synthetic primer
tctggttactgccagttgaatctg

SEQ ID NO: 5 - DNA, 20 bp, Artificial Sequence
  Description of Artificial Sequence Synthetic primer
ttacaaacattggccgcaaa

SEQ ID NO: 6 - DNA, 18 bp, Artificial Sequence
  Description of Artificial Sequence Synthetic primer
gcgcgacattccgaagaa

SEQ ID NO: 7 - DNA, 22 bp, Artificial Sequence
  Description of Artificial Sequence Synthetic primer
gggagccttgaatacaccaaaa

SEQ ID NO: 8 - DNA, 21 bp, Artificial Sequence
  Description of Artificial Sequence Synthetic primer
tgtagcacgattgcagcattg

SEQ ID NO: 9 - DNA, 22 bp, Artificial Sequence
  Description of Artificial Sequence Synthetic primer
gtgaratggtcatgtgtggcgg

SEQ ID NO: 10 - DNA, 26 bp, Artificial Sequence
  Description of Artificial Sequence Synthetic primer
caratgttaaasacactattagcata

SEQ ID NO: 11 - DNA, 26 bp, Artificial Sequence
  Description of Artificial Sequence Synthetic primer
acaggtacgttaatagttaatagcgt

SEQ ID NO: 12 - DNA, 22 bp, Artificial Sequence
  Description of Artificial Sequence Synthetic primer
atattgcagcagtacgcacaca

SEQ ID NO: 13 - DNA, 21 bp, Artificial Sequence
  Description of Artificial Sequence Synthetic primer
ccctgtgggttttacacttaa

SEQ ID NO: 14 - DNA, 19 bp, Artificial Sequence
  Description of Artificial Sequence Synthetic primer
acgattgtgcatcagctga

SEQ ID NO: 15 - DNA, 22 bp, Artificial Sequence
  Description of Artificial Sequence Synthetic primer
gtactcattcgtttcggaagag

SEQ ID NO: 16 - DNA, 22 bp, Artificial Sequence
  Description of Artificial Sequence Synthetic primer
ccagaagatcaggaactctaga

SEQ ID NO: 17 - DNA, 22 bp, Artificial Sequence
  Description of Artificial Sequence Synthetic primer
ggggaacttctcctgctagaat

SEQ ID NO: 18 - DNA, 22 bp, Artificial Sequence
  Description of Artificial Sequence Synthetic primer
cagacattttgctctcaagctg

SEQ ID NO: 19 - DNA, 19 bp, Artificial Sequence
  Description of Artificial Sequence Synthetic primer
agatttggacctgcgagcg

SEQ ID NO: 20 - DNA, 20 bp, Artificial Sequence
  Description of Artificial Sequence Synthetic primer
gagcggctgtctccacaagt

SEQ ID NO: 21 - DNA, 22 bp, Artificial Sequence
  Description of Artificial Sequence Synthetic oligonucleotide
acactgacgacatggttctaca

SEQ ID NO: 22 - DNA, 22 bp, Artificial Sequence
  Description of Artificial Sequence Synthetic oligonucleotide
tacggtagcagagacttggtct

SEQ ID NO: 23 - DNA, 10 bp, Artificial Sequence
  Description of Artificial Sequence Synthetic oligonucleotide
cttacgaact

SEQ ID NO: 24 - DNA, 10 bp, Artificial Sequence
  Description of Artificial Sequence Synthetic oligonucleotide
tgcaacacta

SEQ ID NO: 25 - DNA, 10 bp, Artificial Sequence
  Description of Artificial Sequence Synthetic oligonucleotide
ccagtattct

SEQ ID NO: 26 - DNA, 10 bp, Artificial Sequence
  Description of Artificial Sequence Synthetic oligonucleotide
gcgccatata

SEQ ID NO: 27 - DNA, 10 bp, Artificial Sequence
  Description of Artificial Sequence Synthetic oligonucleotide
ctggagaata

SEQ ID NO: 28 - DNA, 10 bp, Artificial Sequence
  Description of Artificial Sequence Synthetic oligonucleotide
aaccggcctt

SEQ ID NO: 29 - DNA, 10 bp, Artificial Sequence
  Description of Artificial Sequence Synthetic oligonucleotide
agaaccggac

SEQ ID NO: 30 - DNA, 10 bp, Artificial Sequence
  Description of Artificial Sequence Synthetic oligonucleotide
ttggctccaa

SEQ ID NO: 31 - DNA, 10 bp, Artificial Sequence
  Description of Artificial Sequence Synthetic oligonucleotide
aagcggtgaa

SEQ ID NO: 32 - DNA, 10 bp, Artificial Sequence
  Description of Artificial Sequence Synthetic oligonucleotide
cgacgtacac

SEQ ID NO: 33 - DNA, 10 bp, Artificial Sequence
  Description of Artificial Sequence Synthetic oligonucleotide
attcttgcct

SEQ ID NO: 34 - DNA, 10 bp, Artificial Sequence
  Description of Artificial Sequence Synthetic oligonucleotide
cggctaacca

SEQ ID NO: 35 - DNA, 10 bp, Artificial Sequence
  Description of Artificial Sequence Synthetic oligonucleotide
cgtaagagaa

SEQ ID NO: 36 - DNA, 10 bp, Artificial Sequence
  Description of Artificial Sequence Synthetic oligonucleotide
gatgaaggat

SEQ ID NO: 37 - DNA, 10 bp, Artificial Sequence
  Description of Artificial Sequence Synthetic oligonucleotide
gaatgtccaa

SEQ ID NO: 38 - DNA, 10 bp, Artificial Sequence
  Description of Artificial Sequence Synthetic oligonucleotide
tcaccaggac

SEQ ID NO: 39 - DNA, 10 bp, Artificial Sequence
  Description of Artificial Sequence Synthetic oligonucleotide
aagttaggac

SEQ ID NO: 40 - DNA, 10 bp, Artificial Sequence
  Description of Artificial Sequence Synthetic oligonucleotide
aagagcacct

SEQ ID NO: 41 - DNA, 10 bp, Artificial Sequence
  Description of Artificial Sequence Synthetic oligonucleotide
gtgccacaac

SEQ ID NO: 42 - DNA, 10 bp, Artificial Sequence
  Description of Artificial Sequence Synthetic oligonucleotide
aggttcacac

SEQ ID NO: 43 - DNA, 10 bp, Artificial Sequence
  Description of Artificial Sequence Synthetic oligonucleotide
ttgaaggaac

SEQ ID NO: 44 - DNA, 10 bp, Artificial Sequence
  Description of Artificial Sequence Synthetic oligonucleotide
ttaacacgct

SEQ ID NO: 45 - DNA, 10 bp, Artificial Sequence
  Description of Artificial Sequence Synthetic oligonucleotide
tgctggtata

SEQ ID NO: 46 - DNA, 10 bp, Artificial Sequence
  Description of Artificial Sequence Synthetic oligonucleotide
tggagccgat

SEQ ID NO: 47 - DNA, 10 bp, Artificial Sequence
  Description of Artificial Sequence Synthetic oligonucleotide
attacctctc

SEQ ID NO: 48 - DNA, 10 bp, Artificial Sequence
  Description of Artificial Sequence Synthetic oligonucleotide
ccttatggtt

SEQ ID NO: 49 - DNA, 10 bp, Artificial Sequence
  Description of Artificial Sequence Synthetic oligonucleotide
gaggagataa

SEQ ID NO: 50 - DNA, 10 bp, Artificial Sequence
  Description of Artificial Sequence Synthetic oligonucleotide
cttgcactca

SEQ ID NO: 51 - DNA, 10 bp, Artificial Sequence
  Description of Artificial Sequence Synthetic oligonucleotide
gttcctagat

SEQ ID NO: 52 - DNA, 10 bp, Artificial Sequence
  Description of Artificial Sequence Synthetic oligonucleotide
gtaaggtgtt

SEQ ID NO: 53 - DNA, 10 bp, Artificial Sequence
  Description of Artificial Sequence Synthetic oligonucleotide
cggtgttcct

SEQ ID NO: 54 - DNA, 10 bp, Artificial Sequence
  Description of Artificial Sequence Synthetic oligonucleotide
gatatacgca

SEQ ID NO: 55 - DNA, 10 bp, Artificial Sequence
  Description of Artificial Sequence Synthetic oligonucleotide
tggctattac

SEQ ID NO: 56 - DNA, 10 bp, Artificial Sequence
  Description of Artificial Sequence Synthetic oligonucleotide
tgttctcttc

SEQ ID NO: 57 - DNA, 10 bp, Artificial Sequence
  Description of Artificial Sequence Synthetic oligonucleotide
ttccatggta

SEQ ID NO: 58 - DNA, 10 bp, Artificial Sequence
  Description of Artificial Sequence Synthetic oligonucleotide
acctccttct

SEQ ID NO: 59 - DNA, 10 bp, Artificial Sequence
  Description of Artificial Sequence Synthetic oligonucleotide
gagttggctc

SEQ ID NO: 60 - DNA, 10 bp, Artificial Sequence
  Description of Artificial Sequence Synthetic oligonucleotide
caagtgcgtc

SEQ ID NO: 61 - DNA, 10 bp, Artificial Sequence
  Description of Artificial Sequence Synthetic oligonucleotide
ccttggtgca

SEQ ID NO: 62 - DNA, 10 bp, Artificial Sequence
  Description of Artificial Sequence Synthetic oligonucleotide
aggaattgtc

SEQ ID NO: 63 - DNA, 10 bp, Artificial Sequence
  Description of Artificial Sequence Synthetic oligonucleotide
cctgtgtaac

SEQ ID NO: 64 - DNA, 10 bp, Artificial Sequence
  Description of Artificial Sequence Synthetic oligonucleotide
caacaaggca

SEQ ID NO: 65 - DNA, 10 bp, Artificial Sequence
  Description of Artificial Sequence Synthetic oligonucleotide
ttgtggcgca

SEQ ID NO: 66 - DNA, 10 bp, Artificial Sequence
  Description of Artificial Sequence Synthetic oligonucleotide
aattcgcgct

SEQ ID NO: 67 - DNA, 10 bp, Artificial Sequence
  Description of Artificial Sequence Synthetic oligonucleotide
cggtaatatc

SEQ ID NO: 68 - DNA, 10 bp, Artificial Sequence
  Description of Artificial Sequence Synthetic oligonucleotide
attggtggta

SEQ ID NO: 69 - DNA, 10 bp, Artificial Sequence
  Description of Artificial Sequence Synthetic oligonucleotide
gcctcaactc

SEQ ID NO: 70 - DNA, 10 bp, Artificial Sequence
  Description of Artificial Sequence Synthetic oligonucleotide
agaacgcaat

SEQ ID NO: 71 - DNA, 10 bp, Artificial Sequence
  Description of Artificial Sequence Synthetic oligonucleotide
acaagacaac

SEQ ID NO: 72 - DNA, 10 bp, Artificial Sequence
  Description of Artificial Sequence Synthetic oligonucleotide
ctgtcaagtt

SEQ ID NO: 73 - DNA, 10 bp, Artificial Sequence
  Description of Artificial Sequence Synthetic oligonucleotide
accattacca

SEQ ID NO: 74 - DNA, 10 bp, Artificial Sequence
  Description of Artificial Sequence Synthetic oligonucleotide
gtggtgagac

SEQ ID NO: 75 - DNA, 10 bp, Artificial Sequence
  Description of Artificial Sequence Synthetic oligonucleotide
ttagaccttc

SEQ ID NO: 76 - DNA, 10 bp, Artificial Sequence
  Description of Artificial Sequence Synthetic oligonucleotide
gatacgactt

SEQ ID NO: 77 - DNA, 10 bp, Artificial Sequence
  Description of Artificial Sequence Synthetic oligonucleotide
cggattggaa

SEQ ID NO: 78 - DNA, 10 bp, Artificial Sequence
  Description of Artificial Sequence Synthetic oligonucleotide
agcgaggatt

SEQ ID NO: 79 - DNA, 10 bp, Artificial Sequence
  Description of Artificial Sequence Synthetic oligonucleotide
gccagaataa

SEQ ID NO: 80 - DNA, 10 bp, Artificial Sequence
  Description of Artificial Sequence Synthetic oligonucleotide
tgttcagcaa

SEQ ID NO: 81 - DNA, 10 bp, Artificial Sequence
  Description of Artificial Sequence Synthetic oligonucleotide
aacaccggtt

SEQ ID NO: 82 - DNA, 10 bp, Artificial Sequence
  Description of Artificial Sequence Synthetic oligonucleotide
ctccagcttc

SEQ ID NO: 83 - DNA, 10 bp, Artificial Sequence
  Description of Artificial Sequence Synthetic oligonucleotide
tcttaccgaa

SEQ ID NO: 84 - DNA, 10 bp, Artificial Sequence
  Description of Artificial Sequence Synthetic oligonucleotide
ctccaatcct

SEQ ID NO: 85 - DNA, 10 bp, Artificial Sequence
  Description of Artificial Sequence Synthetic oligonucleotide
acactctcaa

SEQ ID NO: 86 - DNA, 10 bp, Artificial Sequence
  Description of Artificial Sequence Synthetic oligonucleotide
gaccgcgtta

SEQ ID NO: 87 - DNA, 10 bp, Artificial Sequence
  Description of Artificial Sequence Synthetic oligonucleotide
tacggcagaa

SEQ ID NO: 88 - DNA, 10 bp, Artificial Sequence
  Description of Artificial Sequence Synthetic oligonucleotide
cacatggaat

SEQ ID NO: 89 - DNA, 10 bp, Artificial Sequence
  Description of Artificial Sequence Synthetic oligonucleotide
tatgccggca

SEQ ID NO: 90 - DNA, 10 bp, Artificial Sequence
  Description of Artificial Sequence Synthetic oligonucleotide
atcgccacct

SEQ ID NO: 91 - DNA, 10 bp, Artificial Sequence
  Description of Artificial Sequence Synthetic oligonucleotide
aagccgaatc

SEQ ID NO: 92 - DNA, 10 bp, Artificial Sequence
  Description of Artificial Sequence Synthetic oligonucleotide
agagcttgtt

SEQ ID NO: 93 - DNA, 10 bp, Artificial Sequence
  Description of Artificial Sequence Synthetic oligonucleotide
tcctgacaat

SEQ ID NO: 94 - DNA, 10 bp, Artificial Sequence
  Description of Artificial Sequence Synthetic oligonucleotide
agaggcctca

SEQ ID NO: 95 - DNA, 10 bp, Artificial Sequence
  Description of Artificial Sequence Synthetic oligonucleotide
ttcgttgaac

SEQ ID NO: 96 - DNA, 10 bp, Artificial Sequence
  Description of Artificial Sequence Synthetic oligonucleotide
gagtactgta

SEQ ID NO: 97 - DNA, 10 bp, Artificial Sequence
  Description of Artificial Sequence Synthetic oligonucleotide
gcacagtcct

SEQ ID NO: 98 - DNA, 10 bp, Artificial Sequence
  Description of Artificial Sequence Synthetic oligonucleotide
ctcatgtcta

SEQ ID NO: 99 - DNA, 10 bp, Artificial Sequence
  Description of Artificial Sequence Synthetic oligonucleotide
tcgattgctt

SEQ ID NO: 100 - DNA, 10 bp, Artificial Sequence
  Description of Artificial Sequence Synthetic oligonucleotide
aatagagcca

SEQ ID NO: 101 - DNA, 10 bp, Artificial Sequence
  Description of Artificial Sequence Synthetic oligonucleotide
atcctccgac

SEQ ID NO: 102 - DNA, 10 bp, Artificial Sequence
  Description of Artificial Sequence Synthetic oligonucleotide
gaagcttact

SEQ ID NO: 103 - DNA, 10 bp, Artificial Sequence
  Description of Artificial Sequence Synthetic oligonucleotide
tgccttggct

SEQ ID NO: 104 - DNA, 10 bp, Artificial Sequence
  Description of Artificial Sequence Synthetic oligonucleotide
attggaacac

SEQ ID NO: 105 - DNA, 10 bp, Artificial Sequence
  Description of Artificial Sequence Synthetic oligonucleotide
aaccaaccaa

SEQ ID NO: 106 - DNA, 10 bp, Artificial Sequence
  Description of Artificial Sequence Synthetic oligonucleotide
aggttacctt

SEQ ID NO: 107 - DNA, 10 bp, Artificial Sequence
  Description of Artificial Sequence Synthetic oligonucleotide
tatggttctc

SEQ ID NO: 108 - DNA, 10 bp, Artificial Sequence
  Description of Artificial Sequence Synthetic oligonucleotide
gaacaatcac

SEQ ID NO: 109 - DNA, 10 bp, Artificial Sequence
  Description of Artificial Sequence Synthetic oligonucleotide
cggtgcttaa

SEQ ID NO: 110 - DNA, 10 bp, Artificial Sequence
  Description of Artificial Sequence Synthetic oligonucleotide
ccgcgtcatt

SEQ ID NO: 111 - DNA, 10 bp, Artificial Sequence
  Description of Artificial Sequence Synthetic oligonucleotide
ccaacagatt

SEQ ID NO: 112 - DNA, 10 bp, Artificial Sequence
  Description of Artificial Sequence Synthetic oligonucleotide
cacacttatc

SEQ ID NO: 113 - DNA, 10 bp, Artificial Sequence
  Description of Artificial Sequence Synthetic oligonucleotide
ctaatccaca

SEQ ID NO: 114 - DNA, 10 bp, Artificial Sequence
  Description of Artificial Sequence Synthetic oligonucleotide
gtcttccatt

SEQ ID NO: 115 - DNA, 10 bp, Artificial Sequence
  Description of Artificial Sequence Synthetic oligonucleotide
tctcggagtt

SEQ ID NO: 116 - DNA, 10 bp, Artificial Sequence
  Description of Artificial Sequence Synthetic oligonucleotide
ctctcgataa

SEQ ID NO: 117 - DNA, 10 bp, Artificial Sequence
  Description of Artificial Sequence Synthetic oligonucleotide
gccaatgttc

SEQ ID NO: 118 - DNA, 10 bp, Artificial Sequence
  Description of Artificial Sequence Synthetic oligonucleotide
tcacttcgca

SEQ ID NO: 119 - DNA, 136 bp, Artificial Sequence
  Description of Artificial Sequence Synthetic polynucleotide
  modified_base (23)..(32): a, c, t, g, unknown or other
  modified_base (105)..(114): a, c, t, g, unknown or other
acactgacgacatggttctacannnnnnnnnngaccccaaaatcagcgaaatgcaccccgcattacgtttggtggaccctcagattcaactggcagtaaccagannnnnnnnnnagaccaagtctctgctaccgta

SEQ ID NO: 120 - DNA, 136 bp, Artificial Sequence; identical to SEQ ID NO: 119
  Description of Artificial Sequence Synthetic polynucleotide
  modified_base (23)..(32): a, c, t, g, unknown or other
  modified_base (105)..(114): a, c, t, g, unknown or other

SEQ ID NO: 121 - DNA, 131 bp, Artificial Sequence
  Description of Artificial Sequence Synthetic polynucleotide
  modified_base (23)..(32): a, c, t, g, unknown or other
  modified_base (100)..(109): a, c, t, g, unknown or other
acactgacgacatggttctacannnnnnnnnnttacaaacattggccgcaaattgcacaatttgcccccagcgcttcagcgttcttcggaatgtcgcgcnnnnnnnnnnagaccaagtctctgctaccgta

SEQ ID NO: 122 - DNA, 131 bp, Artificial Sequence; identical to SEQ ID NO: 121
  Description of Artificial Sequence Synthetic polynucleotide
  modified_base (23)..(32): a, c, t, g, unknown or other
  modified_base (100)..(109): a, c, t, g, unknown or other

SEQ ID NO: 123 - DNA, 285 bp, Artificial Sequence
  Description of Artificial Sequence Synthetic polynucleotide
  modified_base (23)..(32): a, c, t, g, unknown or other
  modified_base (254)..(263): a, c, t, g, unknown or other
acactgacgacatggttctacannnnnnnnnngtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggnnnnnnnnnnagaccaagtctctgctaccgta

SEQ ID NO: 124 - DNA, 285 bp, Artificial Sequence; identical to SEQ ID NO: 123
  Description of Artificial Sequence Synthetic polynucleotide
  modified_base (23)..(32): a, c, t, g, unknown or other
  modified_base (254)..(263): a, c, t, g, unknown or other

SEQ ID NO: 125 - DNA, 163 bp, Artificial Sequence
  Description of Artificial Sequence Synthetic polynucleotide
  modified_base (23)..(32): a, c, t, g, unknown or other
  modified_base (132)..(141): a, c, t, g, unknown or other
acactgacgacatggttctacannnnnnnnnnggggaacttctcctgctagaatggctggcaatggcggtgatgctgctcttgctttgctgctgcttgacagattgaaccagcttgagagcaaaatgtctgnnnnnnnnnnagaccaagtctctgctaccgta

SEQ ID NO: 126 - DNA, 163 bp, Artificial Sequence; identical to SEQ ID NO: 125
  Description of Artificial Sequence Synthetic polynucleotide
  modified_base (23)..(32): a, c, t, g, unknown or other
  modified_base (132)..(141): a, c, t, g, unknown or other

SEQ ID NO: 127 - DNA, 129 bp, Artificial Sequence
  Description of Artificial Sequence Synthetic polynucleotide
  modified_base (23)..(32): a, c, t, g, unknown or other
  modified_base (98)..(107): a, c, t, g, unknown or other
acactgacgacatggttctacannnnnnnnnnagatttggacctgcgagcgggttctgacctgaaggctctgcgcggacttgtggagacagccgctcnnnnnnnnnnagaccaagtctctgctaccgta

SEQ ID NO: 128 - DNA, 129 bp, Artificial Sequence; identical to SEQ ID NO: 127
  Description of Artificial Sequence Synthetic polynucleotide
  modified_base (23)..(32): a, c, t, g, unknown or other
  modified_base (98)..(107): a, c, t, g, unknown or other

SEQ ID NO: 129 - DNA, 125 bp, Artificial Sequence
  Description of Artificial Sequence Synthetic polynucleotide
  modified_base (23)..(32): a, c, t, g, unknown or other
  modified_base (94)..(103): a, c, t, g, unknown or other
acactgacgacatggttctacannnnnnnnnntgctcgcggatacccgtacctcgggtttccgtcttgctcgtatcgctcgagaacgcaagttnnnnnnnnnnagaccaagtctctgctaccgta

SEQ ID NO: 130 - DNA, 125 bp, Artificial Sequence; identical to SEQ ID NO: 129
  Description of Artificial Sequence Synthetic polynucleotide
  modified_base (23)..(32): a, c, t, g, unknown or other
  modified_base (94)..(103): a, c, t, g, unknown or other

SEQ ID NO: 131 - DNA, 266 bp, Artificial Sequence
  Description of Artificial Sequence Synthetic polynucleotide
  modified_base (23)..(32): a, c, t, g, unknown or other
  modified_base (235)..(244): a, c, t, g, unknown or other
acactgacgacatggttctacannnnnnnnnnggctgctcgcggatacccgtacctcgggtttccgtcttgctcgtatcgctcgagaacgcaagttcttcagcgaaaagcacgacagtggtcgctacatagcgtggttccatactggaggtgaaatcaccgacagcatgaagtccgccggcgtgcgcgttatacgcacttcggagtggctaacgccggttcccacattccctcannnnnnnnnnagaccaagtctctgctaccgta

SEQ ID NO: 132 - DNA, 266 bp, Artificial Sequence; identical to SEQ ID NO: 131
  Description of Artificial Sequence Synthetic polynucleotide
  modified_base (23)..(32): a, c, t, g, unknown or other
  modified_base (235)..(244): a, c, t, g, unknown or other

SEQ ID NO: 133 - DNA, 11 bp, Artificial Sequence
  Description of Artificial Sequence Synthetic oligonucleotide
atcaaggcgat

SEQ ID NO: 134 - DNA, 72 bp, Artificial Sequence
  Description of Artificial Sequence Synthetic oligonucleotide
gaccccaaaatcagcgaaatgcaccccgcattacgtttggtggaccctcagattcaactggcagtaaccaga

SEQ ID NO: 135 - DNA, 92 bp, Artificial Sequence
  Description of Artificial Sequence Synthetic oligonucleotide
taacttggtcgaccccaaaatcagcgaaatgcaccccgcattacgtttggtggaccctcagattcaactggcagtaaccagagaccaagtta

SEQ ID NO: 136 - DNA, 10 bp, Artificial Sequence
  Description of Artificial Sequence Synthetic oligonucleotide
taacttggtc

SEQ ID NO: 137 - DNA, 10 bp, Artificial Sequence
  Description of Artificial Sequence Synthetic oligonucleotide
ataagcggag

SEQ ID NO: 138 - DNA, 10 bp, Artificial Sequence
  Description of Artificial Sequence Synthetic oligonucleotide
cgaccttaat

SEQ ID NO: 139 - DNA, 10 bp, Artificial Sequence
  Description of Artificial Sequence Synthetic oligonucleotide
atgttgccac

SEQ ID NO: 140 - DNA, 10 bp, Artificial Sequence
  Description of Artificial Sequence Synthetic oligonucleotide
cagcaggtca

SEQ ID NO: 141 - DNA, 10 bp, Artificial Sequence
  Description of Artificial Sequence Synthetic oligonucleotide
tctagctgcg

SEQ ID NO: 142 - DNA, 10 bp, Artificial Sequence
  Description of Artificial Sequence Synthetic oligonucleotide
agattagcgt

SEQ ID NO: 143 - DNA, 10 bp, Artificial Sequence
  Description of Artificial Sequence Synthetic oligonucleotide
aggtaacaac

SEQ ID NO: 144 - DNA, 10 bp, Artificial Sequence
  Description of Artificial Sequence Synthetic oligonucleotide
gaccaagtta

SEQ ID NO: 145 - DNA, 10 bp, Artificial Sequence
  Description of Artificial Sequence Synthetic oligonucleotide
ctccgcttat

SEQ ID NO: 146 - DNA, 10 bp, Artificial Sequence
  Description of Artificial Sequence Synthetic oligonucleotide
attaaggtcg

SEQ ID NO: 147 - DNA, 10 bp, Artificial Sequence
  Description of Artificial Sequence Synthetic oligonucleotide
gtggcaacat

SEQ ID NO: 148 - DNA, 10 bp, Artificial Sequence
  Description of Artificial Sequence Synthetic oligonucleotide
tgacctgctg

SEQ ID NO: 149 - DNA, 10 bp, Artificial Sequence
  Description of Artificial Sequence Synthetic oligonucleotide
cgcagctaga

SEQ ID NO: 150 - DNA, 10 bp, Artificial Sequence
  Description of Artificial Sequence Synthetic oligonucleotide
acgctaatct

SEQ ID NO: 151 - DNA, 10 bp, Artificial Sequence
  Description of Artificial Sequence Synthetic oligonucleotide
gttgttacct

SEQ ID NO: 152 - DNA, 135 bp, Artificial Sequence
  Description of Artificial Sequence Synthetic polynucleotide
  modified_base (22)..(31): a, c, t, g, unknown or other
  modified_base (104)..(113): a, c, t, g, unknown or other
cactgacgacatggttctacannnnnnnnnngaccccaaaatcagcgaaatgcaccccgcattacgtttggtggaccctcagattcaactggcagtaaccagannnnnnnnnnagaccaagtctctgctaccgta

SEQ ID NO: 153 - DNA, 135 bp, Artificial Sequence
  Description of Artificial Sequence Synthetic polynucleotide
cactgacgacatggttctacattttttttttgaccccaaaatcagcgaaatgcaccccgcattacgtttggtggaccctcagattcaactggcagtaaccagattttttttttagaccaagtctctgctaccgta

SEQ ID NO: 154 - DNA, 135 bp, Artificial Sequence
  Description of Artificial Sequence Synthetic polynucleotide
  modified_base (23)..(25): a, c, t, g, unknown or other
  modified_base (28)..(29): a, c, t, g, unknown or other
  modified_base (31)..(31): a, c, t, g, unknown or other
  modified_base (104)..(110): a, c, t, g, unknown or other
  modified_base (113)..(113): a, c, t, g, unknown or other
cactgacgacatggttctacatnnnttnntngaccccaaaatcagcgaaatgcaccccgcattacgtttggtggaccctcagattcaactggcagtaaccagannnnnnnttnagaccaagtctctgctaccgta

SEQ ID NO: 155 - DNA, 135 bp, Artificial Sequence
  Description of Artificial Sequence Synthetic polynucleotide
cactgacgacatggttctacataacttggtcgaccccaaaatcagcgaaatgcaccccgcattacgtttggtggaccctcagattcaactggcagtaaccagagaccaagttaagaccaagtctctgctaccgta

SEQ ID NO: 156 - DNA, 128 bp, Artificial Sequence
  Description of Artificial Sequence Synthetic polynucleotide
  modified_base (23)..(35): a, c, t, g, unknown or other
acactgacgacatggttctacannnnnnnnnnnnngtactcattcgtttcggaagagacaggtacgttaatagttaatagcgtacttctttttcttgctttcgtggtattcttgctagttacactagc

SEQ ID NO: 157 - DNA, 128 bp, Artificial Sequence
  Description of Artificial Sequence Synthetic polynucleotide
  modified_base (31)..(33): a, c, t, g, unknown or other
acactgacgacatggttctacattttttttnnnttgtactcattcgtttcggaagagacaggtacgttaatagttaatagcgtacttctttttcttgctttcgtggtattcttgctagttacactagc

SEQ ID NO: 158 - DNA, 128 bp, Artificial Sequence
  Description of Artificial Sequence Synthetic polynucleotide
  modified_base (23)..(28): a, c, t, g, unknown or other
  modified_base (30)..(35): a, c, t, g, unknown or other
  modified_base (37)..(37): a, c, t, g, unknown or other
  modified_base (41)..(42): a, c, t, g, unknown or other
  modified_base (44)..(45): a, c, t, g, unknown or other
  modified_base (47)..(47): a, c, t, g, unknown or other
  modified_base (49)..(51): a, c, t, g, unknown or other
  modified_base (53)..(54): a, c, t, g, unknown or other
  modified_base (57)..(57): a, c, t, g, unknown or other
  modified_base (62)..(75): a, c, t, g, unknown or other
  modified_base (79)..(79): a, c, t, g, unknown or other
  modified_base (83)..(86): a, c, t, g, unknown or other
  modified_base (88)..(89): a, c, t, g, unknown or other
  modified_base (91)..(92): a, c, t, g, unknown or other
  modified_base (102)..(103): a, c, t, g, unknown or other
  modified_base (106)..(106): a, c, t, g, unknown or other
  modified_base (108)..(109): a, c, t, g, unknown or other
  modified_base (112)..(112): a, c, t, g, unknown or other
  modified_base (115)..(118): a, c, t, g, unknown or other
  modified_base (121)..(122): a, c, t, g, unknown or other
  modified_base (125)..(125): a, c, t, g, unknown or other
acactgacgacatggttctacannnnnntnnnnnngnactnntnngntnnngnnganacagnnnnnnnnnnnnnnaatngcgnnnntnntnntcttgctttnntgntnntcntgnnnnttnnacnagc

SEQ ID NO: 159 - DNA, 128 bp, Artificial Sequence
  Description of Artificial Sequence Synthetic polynucleotide
  modified_base (62)..(74): a, c, t, g, unknown or other
acactgacgacatggttctacacaagactcaaggggaacttctctgctagaggtgaaacagnnnnnnnnnnnnncaatggcggtgatgctgctcttgctttgctgctgctcctgtggattgaaccagc

SEQ ID NO: 160 - DNA, 24 bp, Artificial Sequence
  Description of Artificial Sequence Synthetic oligonucleotide
cacaaagacaccgacaactttctt

SEQ ID NO: 161 - DNA, 24 bp, Artificial Sequence
  Description of Artificial Sequence Synthetic oligonucleotide
acagacgactacaaacggaatcga

SEQ ID NO: 162 - DNA, 24 bp, Artificial Sequence
  Description of Artificial Sequence Synthetic oligonucleotide
cctggtaactgggacacaagactc

SEQ ID NO: 163 - DNA, 24 bp, Artificial Sequence
  Description of Artificial Sequence Synthetic oligonucleotide
tagggaaacacgatagaatccgaa

SEQ ID NO: 164 - DNA, 24 bp, Artificial Sequence
  Description of Artificial Sequence Synthetic oligonucleotide
aaggttacacaaaccctggacaag

SEQ ID NO: 165 - DNA, 24 bp, Artificial Sequence
  Description of Artificial Sequence Synthetic oligonucleotide
gactactttctgcctttgcgagaa

SEQ ID NO: 166 - DNA, 24 bp, Artificial Sequence
  Description of Artificial Sequence Synthetic oligonucleotide
aaggattcattcccacggtaacac

SEQ ID NO: 167 - DNA, 24 bp, Artificial Sequence
  Description of Artificial Sequence Synthetic oligonucleotide
acgtaacttggtttgttccctgaa

SEQ ID NO: 168 - DNA, 24 bp, Artificial Sequence
  Description of Artificial Sequence Synthetic oligonucleotide
aaccaagactcgctgtgcctagtt

SEQ ID NO: 169 - DNA, 24 bp, Artificial Sequence
  Description of Artificial Sequence Synthetic oligonucleotide
gagaggacaaaggtttcaacgctt

SEQ ID NO: 170 - DNA, 24 bp, Artificial Sequence
  Description of Artificial Sequence Synthetic oligonucleotide
tccattccctccgatagatgaaac

SEQ ID NO: 171 - DNA, 24 bp, Artificial Sequence
  Description of Artificial Sequence Synthetic oligonucleotide
tccgattctgcttctttctacctg

SEQ ID NO: 172 - DNA, 24 bp, Artificial Sequence
  Description of Artificial Sequence Synthetic oligonucleotide
tcacacgagtatggaagtcgttct

SEQ ID NO: 173 - DNA, 24 bp, Artificial Sequence
  Description of Artificial Sequence Synthetic oligonucleotide
tctatgggtcccaagagactcgtt

SEQ ID NO: 174 - DNA, 24 bp, Artificial Sequence
  Description of Artificial Sequence Synthetic oligonucleotide
cagtggtgttagcgaggtagacct

SEQ ID NO: 175 - DNA, 24 bp, Artificial Sequence
  Description of Artificial Sequence Synthetic oligonucleotide
agtacgaaccactgtcagttgacg

SEQ ID NO: 176 - DNA, 24 bp, Artificial Sequence
  Description of Artificial Sequence Synthetic oligonucleotide
atcagaggtactttcctggagggt

SEQ ID NO: 177 - DNA, 24 bp, Artificial Sequence
  Description of Artificial Sequence Synthetic oligonucleotide
gcctatctaggttgttgggtttgg

SEQ ID NO: 178 - DNA, 24 bp, Artificial Sequence
  Description of Artificial Sequence Synthetic oligonucleotide
atctcttgacactgcacgaggaac

SEQ ID NO: 179 - DNA, 24 bp, Artificial Sequence
  Description of Artificial Sequence Synthetic oligonucleotide
atgagttctcgtaacaggacgcaa

SEQ ID NO: 180 - DNA, 24 bp, Artificial Sequence
  Description of Artificial Sequence Synthetic oligonucleotide
tagagaacggacaatgagaggctc

SEQ ID NO: 181 - DNA, 24 bp, Artificial Sequence
  Description of Artificial Sequence Synthetic oligonucleotide
cgtactttgatacatggcagtggt

SEQ ID NO: 182 - DNA, 24 bp, Artificial Sequence
  Description of Artificial Sequence Synthetic oligonucleotide
cgaggaggttcactgggtagtaag

SEQ ID NO: 183 - DNA, 24 bp, Artificial Sequence
  Description of Artificial Sequence Synthetic oligonucleotide
ctaacccatcatgcagaactatgc

SEQ ID NO: 184 - DNA, 24 bp, Artificial Sequence
  Description of Artificial Sequence Synthetic oligonucleotide
aagaaagttgtcggtgtctttgtg

SEQ ID NO: 185 - DNA, 24 bp, Artificial Sequence
  Description of Artificial Sequence Synthetic oligonucleotide
tcgattccgtttgtagtcgtctgt

SEQ ID NO: 186 - DNA, 24 bp, Artificial Sequence
  Description of Artificial Sequence Synthetic oligonucleotide
gagtcttgtgtcccagttaccagg

SEQ ID NO: 187 - DNA, 24 bp, Artificial Sequence
  Description of Artificial Sequence Synthetic oligonucleotide
ttcggattctatcgtgtttcccta

SEQ ID NO: 188 - DNA, 24 bp, Artificial Sequence
  Description of Artificial Sequence Synthetic oligonucleotide
cttgtccagggtttgtgtaacctt

SEQ ID NO: 189 - DNA, 24 bp, Artificial Sequence
  Description of Artificial Sequence Synthetic oligonucleotide
ttctcgcaaaggcagaaagtagtc

SEQ ID NO: 190 - DNA, 24 bp, Artificial Sequence
  Description of Artificial Sequence Synthetic oligonucleotide
gtgttaccgtgggaatgaatcctt

SEQ ID NO: 191 - DNA, 24 bp, Artificial Sequence
  Description of Artificial Sequence Synthetic oligonucleotide
ttcagggaacaaaccaagttacgt

SEQ ID NO: 192 - DNA, 24 bp, Artificial Sequence
  Description of Artificial Sequence Synthetic oligonucleotide
aactaggcacagcgagtcttggtt

SEQ ID NO: 193 - DNA, 24 bp, Artificial Sequence
  Description of Artificial Sequence Synthetic oligonucleotide
aagcgttgaaacctttgtcctctc

SEQ ID NO: 194 - DNA, 24 bp, Artificial Sequence
  Description of Artificial Sequence Synthetic oligonucleotide
gtttcatctatcggagggaatgga

SEQ ID NO: 195 - DNA, 24 bp, Artificial Sequence
  Description of Artificial Sequence Synthetic oligonucleotide
caggtagaaagaagcagaatcgga

SEQ ID NO: 196 - DNA, 24 bp, Artificial Sequence
  Description of Artificial Sequence Synthetic oligonucleotide
agaacgacttccatactcgtgtga

SEQ ID NO: 197 - DNA, 24 bp, Artificial Sequence
  Description of Artificial Sequence Synthetic oligonucleotide
aacgagtctcttgggacccataga

SEQ ID NO: 198 - DNA, 24 bp, Artificial Sequence
  Description of Artificial Sequence Synthetic oligonucleotide
aggtctacctcgctaacaccactg

SEQ ID NO: 199 - DNA, 24 bp, Artificial Sequence
  Description of Artificial Sequence Synthetic oligonucleotide
cgtcaactgacagtggttcgtact

SEQ ID NO: 200 - DNA, 24 bp, Artificial Sequence
  Description of Artificial Sequence Synthetic oligonucleotide
accctccaggaaagtacctctgat

SEQ ID NO: 201 - DNA, 24 bp, Artificial Sequence
  Description of Artificial Sequence Synthetic oligonucleotide
ccaaacccaacaacctagataggc

SEQ ID NO: 202 - DNA, 24 bp, Artificial Sequence
  Description of Artificial Sequence Synthetic oligonucleotide
gttcctcgtgcagtgtcaagagat

SEQ ID NO: 203 - DNA, 24 bp, Artificial Sequence
  Description of Artificial Sequence Synthetic oligonucleotide
ttgcgtcctgttacgagaactcat

SEQ ID NO: 204 - DNA, 24 bp, Artificial Sequence
  Description of Artificial Sequence Synthetic oligonucleotide
gagcctctcattgtccgttctcta

SEQ ID NO: 205 - DNA, 24 bp, Artificial Sequence
  Description of Artificial Sequence Synthetic oligonucleotide
accactgccatgtatcaaagtacg

SEQ ID NO: 206 - DNA, 24 bp, Artificial Sequence
  Description of Artificial Sequence Synthetic oligonucleotide
cttactacccagtgaacctcctcg

SEQ ID NO: 207 - DNA, 24 bp, Artificial Sequence
  Description of Artificial Sequence Synthetic oligonucleotide
gcatagttctgcatgatgggttag